(19) United States
(12) Patent Application Publication
(10) Pub. No.: US 2005/0013818 A1
(43) Pub. Date: Jan. 20, 2005

(54) Compositions and methods for the suppression of mammary epithelial cell proliferation

(75) Inventors: Michael Karin, La Jolla, CA (US); Yixue Cao, San Diego, CA (US)

Correspondence Address: MEDLEN & CARROLL, LLP, Suite 350, 101 Howard Street, San Francisco, CA, 94105, US

(73) Assignee: The Regents of the University of California

(21) Appl. No.: 10/845,854

(22) Filed: May 13, 2004

Related U.S. Application Data
(63) Continuation-in-part of application No. PCT/US02/36674, filed on Nov. 14, 2002.
(60) Provisional application No. 60/334,779, filed on Nov. 15, 2001.

(51) Int. Cl. A61K 39/395; G01N 33/574

(52) U.S. Cl. 424/155.1; 435/7.23

(57) ABSTRACT

The present invention provides compositions and methods for the suppression of mammary epithelial cell proliferation. In particular, the present invention provides compositions and methods for IKKα kinase inhibition in breast cancer therapy that is relatively free of toxic side effects.

This application is a continuation-in-part of, and claims priority to co-pending PCT Application No. PCT/US02/36674, filed on Nov. 14, 2002, which claims priority to U.S. Provisional Application Ser. No. 60/334,779, filed Nov. 15, 2001, now abandoned, the contents of which are incorporated herein in their entirety.

The present invention was made with government support from the National Cancer Institute of the National Institutes of Health, Grant No. A143477. The United States Government has certain rights in the invention.

### FIELD OF THE INVENTION

The present invention provides compositions and methods for the suppression of mammary epithelial cell proliferation. In particular, the present invention provides compositions and methods for specific IKKα kinase inhibition as a breast cancer therapy that is relatively free of toxic side effects.

### BACKGROUND OF THE INVENTION

Breast cancer kills over 45,000 women each year in the United States alone. It is estimated that one in eight women will develop breast cancer. Over 180,000 new cases of breast cancer are diagnosed annually, making breast cancer one of the most dangerous diseases facing women today.

To date, there are no suitable prophylactic or therapeutic approaches to breast cancer. For example, while breast cancer is currently managed by surgery, hormone therapy, chemotherapy, and radiation, these approaches are toxic, dangerous, and costly, and many are ineffective, especially in the treatment of metastatic disease.

Unchecked neoplastic growth of mammary tissues can develop into malignant tumors, which are the cause of death of thousands of women yearly. Despite extensive research in the art, the critical proteins that are involved in various signal transduction pathways leading to mammary epithelial cell proliferation have not been identified. Thus, an understanding of transduction pathways that are involved in the regulation of mammary epithelial cell growth is of great importance for the diagnosis, prevention, and treatment of breast cancer, as well as for rational design of drugs that serve these purposes.

Thus, there remains a need to identify proteins that are involved in signal transduction pathways leading to mammary epithelial cell proliferation. In particular, there is a need for methods and compositions for reducing mammary epithelial cell proliferation, for detecting tumor mammary epithelial cells, and for screening test compounds that reduce mammary epithelial cell proliferation.

### SUMMARY OF THE INVENTION

The present invention provides methods and compositions for reducing mammary epithelial cell proliferation, for detecting tumor mammary epithelial cells, and screening test compounds that reduce mammary epithelial cell proliferation. In particular, the present invention provides compositions and methods involving IKKα. In particularly preferred embodiments, the targeting of IKKα inhibits the proliferation of mammary epithelial cells without inhibiting the proliferation or causing the death of other cell types.

In some embodiments, the present invention provides methods for modifying IKKα activity. In some embodiments, the modifying comprises inhibiting IKKα kinase activities, while in other embodiments, the modifying comprises inhibiting its expression. More particularly, the invention provides a method for reducing mammary epithelial cell proliferation, comprising: a) providing: i) mammary epithelial cells; and ii) an agent that specifically reduces IKKα kinase activity; b) administering the agent to the mammary epithelial cells to produce treated mammary epithelial cells such that mammary epithelial cell proliferation in the treated mammary epithelial cells is reduced. In one embodiment, the mammary epithelial cells are in vitro. In an alternative embodiment, the mammary epithelial cells are in vivo in a subject. In a preferred embodiment, the subject is human or mouse. In an alternative embodiment, the mammary epithelial cells are in a tissue comprising a mammary tumor, more preferably, the tumor is malignant, and even more preferably, the malignant tumor is metastatic. Alternatively, the subject is suspected of being capable of developing a mammary tumor in a tissue. In one embodiment, the administering is before, concomitant with, and/or after manifestation of one or more symptoms of a mammary tumor. While not limiting the route of administration, the administering is selected from parenteral, oral, intraperitoneal, and sublingual, and more preferably the parenteral administering is selected from subcutaneous, intravenous, intramuscular, intrastemal injection, and infusion. In a particularly preferred embodiment, the administering is into the tumor.

While not limiting the type or source of agent, in one embodiment, the agent comprises an antibody that specifically binds to an IKKα kinase domain. Preferably, the agent further comprises a carrier selected from dextran, liposomes, polyethylene glycol, acrylic acid, acrylamide polymers, N-(2-hydroxypropyl) methacrylamide polymers, colloidal gold, virus, zinc sulfide-capped cadmium selenide, phosphor crystals in suspension with polycarboxylic acids, nanoparticles, microparticles, plasmon resonant particles, and polystyrene particles. Alternatively, the agent further comprises a chelator. More preferably, the chelator comprises a molecule selected from tetraazacyclododecanetetraacetic acid (DOTA), mercaptoacetylglycylglycyl-glycine (MAG3), diethylenetriamine pentaacetic acid (DTPA), 4-[2-[(2-mercapto-2-methylpropyl)methylamino]-ethyl]-6,6-dimethyl-2-thiomorpholinone (DADT), and deferoxamine. In an alternative embodiment, the agent further comprises a reporter, preferably, the reporter comprise a molecule selected from technetium-99m (Tc-99m), Indium-111 (In-111), Gallium-68 (Ga-68), Gallium-67 (Ga-67), rhenium-186 (Re-186), rhenium-188 (Re-188), iodine-123 (I-123), iodine-125 (I-125), iodine-131 (I-131), iodine (I), gadolinium (Gd), ytterbium (Yb), dysprosium (Dy), europium (Eu), perflubron-based emulsion, and microbubble-based emulsion. In yet another alternative, the agent further comprises a cytotoxin. The cytotoxxin is preferably a tumor chemotherapeutic compound. Alternatively, the cytotoxin comprises a molecule selected from cAMP, pertussis toxin, Cholera toxin, C3 exoenzyme, Ricin A, etoposide, campothecin irinotecan, topotecan, doxorubicine, daunorubicine, vincristine, vinblastine, vinorelbine, paclitaxel, docetaxel, cisplatin, carboplatin, oxaloplatin, cyclophosphamide, ifosfamide, methotrexate, mercaptopurine, tamoxifen, toremifene, trans-retinoic acid, adriamycin, gemcitabine, 5-fluoruracil, and maytanisoids. In another embodiment, the agent comprises an IKKα kinase domain antisense sequence. Alternatively, the agent comprises an IKKα kinase domain ribozyme.

In some preferred embodiments, the present invention provides means to identify treatment means for the suppression of epithelial cell proliferation. In some particularly preferred embodiments, the present invention provides means to identify treatment means for the suppression of breast epithelial cell proliferation. In alternative preferred embodiments, the present invention provides means to identify treatment means for breast cancer. In still further embodiments, the present invention provides means to identify treatment means for other cancers, in particular those that involve epithelial cell proliferation. More particularly, provided herein is a method for reducing a mammary tumor in a subject, comprising: a) providing: i) a subject; and ii) an agent that specifically reduces IKKα kinase activity; b) administering the agent to the subject to produce a treated subject such that a mammary tumor in the treated subject is reduced. In one embodiment, the subject is suspected of being capable of developing a mammary tumor in a tissue. Alternatively, the subject comprises a mammary tumor in a tissue. In one embodiment, the subject is human or mouse. In another embodiment, the mammary epithelial cells are in a tissue comprising a mammary tumor. Preferably, the tumor is malignant, and more preferably, the malignant tumor is metastatic. In one embodiment, the subject is suspected of being capable of developing a mammary tumor in a tissue. In a preferred embodiment, the administering is before, concomitant with, and/or after manifestation of one or more symptoms of a mammary tumor. In one embodiment, the administering is selected from parenteral, oral, intraperitoneal, and sublingual, and preferably, the parenteral administering is selected from subcutaneous, intravenous, intramuscular, intrastemal injection, and infusion. Most preferably, the administering is into the tumor.

While not limiting the type or source of agent, in one embodiment, the agent comprises an antibody that specifically binds to an IKKα kinase domain. Preferably, the agent further comprises a carrier selected from dextran, liposomes, polyethylene glycol, acrylic acid, acrylamide polymers, N-(2-hydroxypropyl) methacrylamide polymers, colloidal gold, virus, zinc sulfide-capped cadmium selenide, phosphor crystals in suspension with polycarboxylic acids, nanoparticles, microparticles, plasmon resonant particles, and polystyrene particles. Alternatively, the agent further comprises a chelator. More preferably, the chelator comprises a molecule selected from tetraazacyclododecanetetraacetic acid (DOTA), mercaptoacetylglycylglycyl-glycine (MAG3), diethylenetriamine pentaacetic acid (DTPA), 4-[2-[(2-mercapto-2-methylpropyl)methylamino]-ethyl]-6,6-dimethyl-2-thiomorpholinone (DADT), and deferoxamine. In an alternative embodiment, the agent further comprises a reporter, preferably, the reporter comprise a molecule selected from technetium-99m (Tc-99m), Indium-111 (In-111), Gallium-68 (Ga-68), Gallium-67 (Ga-67), rhenium-186 (Re-186), rhenium-188 (Re-188), iodine-123 (I-123), iodine-125 (I-125), iodine-131 (I-131), iodine (I), gadolinium (Gd), ytterbium (Yb), dysprosium (Dy), europium (Eu), perflubron-based emulsion, and microbubble-based emulsion. In yet another alternative, the agent further comprises a cytotoxin. The cytotoxin is preferably a tumor chemotherapeutic compound. Alternatively, the cytotoxin comprises a molecule selected from cAMP, pertussis toxin, Cholera toxin, C3 exoenzyme, Ricin A, etoposide, campothecin irinotecan, topotecan, doxorubicine, daunorubicine, vincristine, vinblastine, vinorelbine, paclitaxel, docetaxel, cisplatin, carboplatin, oxaloplatin, cyclophosphamide, ifosfamide, methotrexate, mercaptopurine, tamoxifen, toremifene, trans-retinoic acid, adriamycin, gemcitabine, 5-fluoruracil, and maytanisoids. In another embodiment, the agent comprises an IKKα kinase domain antisense sequence. Alternatively, the agent comprises an IKKα kinase domain ribozyme.

The invention additionally provides a method for detecting tumor mammary epithelial cells in a sample, comprising detecting increased IKKα kinase activity in the tumor mammary epithelial cells or in the sample compared to IKKα kinase activity in control cells or in a control sample.

Moreover, the invention provides a method for detecting tumor mammary epithelial cells in a sample, comprising: a) providing: i) a sample suspected of comprising tumor mammary epithelial cells; and ii) an anti-phosphoserine antibody; b) administering said antibody to said sample such that said antibody binds to phosphoserine in an IKKα kinase domain to produce a treated sample; and c) detecting increased binding of said antibody to said phosphoserine in said treated sample compared to binding of said antibody to said phosphoserine in a control sample, thereby detecting tumor mammary epithelial cells in said sample. In one embodiment, the tumor mammary epithelial cells are in vitro. In another embodiment, the tumor mammary epithelial cells are in vivo.

The invention also provides method for detecting tumor mammary epithelial cells in a sample, comprising: a) providing: i) a sample suspected of comprising tumor mammary epithelial cells; and ii) an agent that specifically binds to at least a portion of a molecule selected from IKKα polypeptide, IKKα kinase domain polypeptide, IKKα mRNA, and IKKα kinase domain mRNA; b) administering the agent to the sample such that the agent specifically binds to the molecule to produce a treated sample comprising treated tumor mammary epithelial cells; and c) detecting increased specific binding of the molecule to the agent in the treated sample or in the treated cells as compared to specific binding of the molecule to the agent in a control sample or in control cells, thereby detecting tumor mammary epithelial cells in the sample.

While not intending to limit the size or sequence of the portion, in one embodiment, the portion of IKKα polypeptide comprises an IKKα kinase domain. In another embodiment, the portion of IKKα mRNA encodes a polypeptide comprising an IKKα kinase domain.

Without limiting the type or source of the agent, in one embodiment, the agent comprises an anti-IKKα antibody. In a preferred embodiment, the antibody specifically binds to an IKKα kinase domain. In an alternative embodiment, the agent comprises a DNA or RNA sequence that specifically binds to a DNA or RNA sequence encoding IKKα or IKKα kinase domain. In another embodiment, the agent comprises an anti-phosphoserine antibody.

In alternative embodiments, the present invention provides means to determine whether a test compound of interest is capable of modifying IKKα activity. In some embodiments, the test compound comprises a pharmaceutical, while in other embodiments, the test compound comprises antisense oligonucleotides or other genetic sequences. More particularly, the invention additionally provides a method for screening a test compound as reducing mammary epithelial cell proliferation, comprising: a) providing: i) mammary epithelial cells; ii) a test compound; and b) administering the test compound to the mammary epithelial cells to produce treated mammary epithelial cells; c) detecting a reduction in IKKα kinase activity in the treated mammary epithelial cells, thereby identifying the test compound as reducing mammary epithelial cell proliferation. In one embodiment, the mammary epithelial cells are in vitro, and in another, the mammary epithelial cells are in vivo. In a preferred embodiment, the mammary epithelial cells are normal. Preferably, the normal mammary epithelial cells are a cell line, and more preferably, the normal mammary epithelial cell line is HC11. In an alternative embodiment, the normal mammary epithelial cells are primary cultured cells. In yet another alternative embodiment, the mammary epithelial cells are tumor. In one embodiment, the tumor mammary epithelial cells are a cell line, more preferably, the tumor mammary epithelial cell line is a human tumor mammary epithelial cell lines selected from MCF7, T47D, MDA-MB-231, MDA-MB-435, SKBR3, BT474, and Hs578T, and alternatively, the tumor mammary epithelial cell line is a rodent tumor mammary epithelial cell ling selected from mouse CSMLO, mouse NJH-3T3, and rat RM22-F5. In a further alternative, the tumor mammary epithelial cells are primary cultured cells.

Also provided herein is a method for screening a test compound as reducing mammary epithelial cell proliferation, comprising: a) providing: i) a sample comprising IKKα; ii) an antibody that specifically binds to IKKα kinase domain; and iii) a test compound; b) contacting said sample, said antibody, and said test compound such that said antibody binds to the IKKα kinase domain in said IKKα; and c) detecting a reduction in binding of said antibody to said IKKα kinase domain in the presence of said test compound compared to in the absence of said test compound, thereby identifying said test compound as reducing mammary epithelial cell proliferation. In one embodiment, the antibody is monoclonal, such as the monoclonal antibody from BD PharMingen, San Diego Calif., cat# 556532; several antibodies from Santa Cruz Biotechnology, Inc. Santa Cruz Calif., for example, cat# sc-7606 (monoclonal), and from Imgenex, San Diego Calif., such as cat# IMG-136 (monoclonal). In another embodiment, the antibody is polyclonal, such as those from Santa Cruz Biotechnology, Inc. Santa Cruz Calif., cat# sc-7121 (polyclonal against C-terminus), cat# sc-7218 (polyclonal), cat# sc-7183 (polyclonal against mouse amino acids 465-745), cat# sc-7190 (polyclonal), and cat# sc-7184 (polyclonal against mouse amino acids 248-452).

Also provided herein is a method for screening a test compound as reducing mammary epithelial cell proliferation, comprising: a) providing: i) a sample comprising IKKα or IKKα kinase domain; ii) an IKKα substrate; iii) a test compound; and b) contacting said sample, said IKKα substrate, and said test compound such that said IKKα substrate is phosphorylated by said IKKαor said IKKαkinase domain; and c) detecting a reduction in the level of phosphorylation of said IKKα substrate in the presence of said test compound compared to in the absence of said test compound, thereby identifying said test compound as reducing mammary epithelial cell proliferation. In one embodiment, the IKKα kinase domain is recombinant. In another embodiment, the IKKα in said sample is recombinant, preferably, the recombinant IKKα has the amino acid sequence of SEQ ID NO:13 or SEQ ID NO:15.

The invention also provides a method for screening a test compound as reducing mammary epithelial cell proliferation, comprising: a) providing: i) a sample comprising IKKα; ii) an anti-phosphoserine antibody; iii) a test compound; and b) contacting said sample, said antibody, and said test compound such that said antibody binds to said IKKα; and c) detecting a reduction in the level of binding of said antibody to said IKKα in the presence of said test compound compared to in the absence of said test compound, thereby identifying said test compound as reducing mammary epithelial cell proliferation.

Also provided by the instant invention is a method for screening a test compound as specifically reducing IKKα kinase activity, comprising: a) providing: i) a first transgenic non-human animal selected from mouse, sheep, pig, rabbit and cattle expressing: 1) an oncogene that causes mammary tumors in said first transgenic animal; ii) a second transgenic non-human animal expressing: 1) said oncogene; and 2) a modified IKKα polypeptide having reduced IKKα kinase activity, wherein said second transgenic animal is of the same species as said first transgenic animal; and iii) a test compound; and b) administering said test compound to said first transgenic animal to produce a first treated transgenic animal and to said second transgenic animal to produce a second treated transgenic animal; and c) detecting a reduction in mammary tumors in said first treated transgenic animal that is not greater than a reduction in mammary tumors in said second treated transgenic animal, thereby identifying said test compound as specifically reducing IKKα kinase activity. In one embodiment, the method further comprises identifying said test compound as reducing mammary epithelial cell proliferation. In one embodiment, the non-human animal is a mouse, preferably, the mouse comprises a mutation in the IKKα kinase domain, more preferably, the mutation in the IKKα kinase domain comprises replacing at least one of the Serine residues at amino acids 176 and 180 of IKKα kinase polypeptide with Alanine. Alternatively, the mutation in the IKKα kinase domain comprises replacing Lysine at amino acid 44 of IKKα kinase polypeptide with Alanine or Methionine. In one embodiment, the oncogene is selected from neu, c-myc, and s-src, more preferably the oncogene is the neu oncogene.

The invention also provides A method for screening a test compound as specifically reducing IKKα kinase activity, comprising: a) providing: i) a first transgenic mouse expressing a neu oncogene; ii) a second transgenic mouse expressing: 1) a neu oncogene; and 2) a modified IKKα kinase domain that comprises replacing the Serine residues at amino acids 176 and 180 of IKKα kinase polypeptide with Alanine, wherein said modified IKKα kinase domain has reduced IKKα kinase activity; and iii) a test compound; and b) administering said test compound to said first transgenic mouse to produce a first treated transgenic mouse and to said second transgenic mouse to produce a second treated transgenic mouse; and c) detecting a reduction in mammary tumors in said first treated transgenic mouse that is not greater than a reduction in mammary tumors in said second treated transgenic mouse, thereby identifying said test compound as specifically reducing IKKα kinase activity. In one embodiment, the method further comprises identifying said test compound as reducing mammary epithelial cell proliferation.

In one embodiment, the present invention provides transgenic non-human animals. In some preferred embodiments, the animals are Ikkα^{AA} mutants. In some particularly preferred embodiments, the animals are knockin. In other embodiments, the present invention provides methods for testing of IKKα function in the transgenic non-human animals. More specifically, provided herein is a transgenic non-human animal selected from mouse, sheep, pig, rabbit and cattle expressing a modified IKKαpolypeptide that has reduced IKKα kinase activity, wherein the female of the animal has normal skin and limbs compared to a control female animal. In one embodiment, the animal is a mouse. In another embodiment, the animal comprises a mutation in the IKKα kinase domain, more preferably, the mutation in the IKKα kinase domain comprises replacing Serine at each of amino acids 176 and 180 of IKKα kinase polypeptide with Alanine. In a further embodiment, mutation in the IKKα kinase domain comprises replacing Serine amino acid 176 or amino acid 180 of IKKα kinase polypeptide with Alanine. In a further embodiment, the mutation in the IKKα kinase domain comprises replacing Lysine at amino acid 44 of IKKα kinase polypeptide with Alanine or Methionine. While the type of animal is not intended to be limited, in one embodiment, the animal is chimeric, heterozygous for the mutation, or homozygous for the mutation. In one preferred embodiment, the female of the animal has a reduced mammary epithelial cell proliferation rate during pregnancy, lactation, or both pregnancy and lactation, compared to a control female animal. In another embodiment, the female of the animal has a reduced number of mammary epithelial cells during pregnancy, lactation, or both pregnancy and lactation, compared to a control female animal. In yet another alternative, the female of the animal has the same mammary epithelial cell apoptotic rate compared to a control female animal. In a further alternative, the female of the animal has a reduced level of cyclin D1 polypeptide, cyclin D1 mRNA, or both cyclin D1 polypeptide and cyclin D1 mRNA, compared to a control female animal. In yet a further alternative, the female of the animal has a reduced level of NF-κB activation during pregnancy, lactation, or both pregnancy and lactation, compared to a control female animal. In another alternative embodiment, the female of the animal has the same number of T cells compared to a control female animal.

The invention further provides a transgenic non-human animal selected from mouse, sheep, pig, rabbit and cattle expressing a modified IκBα polypeptide, wherein the female of the transgenic non-human animal has a reduced level of IκBα phosphorylation during pregnancy, lactation, or both pregnancy and lactation, compared to a control animal. In one embodiment, the modified IκBα polypeptide comprises a mutation comprising replacing Serine at each of amino acids 32 and 36 with Alanine. In a preferred embodiment, the female of the animal has a reduced mammary epithelial cell proliferation rate during pregnancy, lactation, or both pregnancy and lactation, compared to a control female animal. In another preferred embodiment, the female of the animal has a reduced level of cyclin D1 polypeptide, cyclin D1 mRNA, or both cyclin D1 polypeptide and cyclin D1 mRNA, compared to a control female animal.

### DESCRIPTION OF THE DRAWINGS

FIG. 1, Panel A provides data obtained during the generation of Ikkα^{AA} mutant mice. Panel A provides a schematic of the Ikkα^{AA} targeting vector and the targeted locus after homologous recombination. Coding exons are depicted by solid boxes. Solid lines represent introns and solid triangles are loxP sites. A Neo^{r} cassette was inserted in the opposite orientation to the Ikkα transcription unit. FIG. 1, Panel B shows results of experiments conducted in genotyping of F₂ mice by PCR analysis of tail DNA obtained from these mice. A forward primer (“p392,” specific for exon 5) was used in combinations with either “pWT,” a primer specific for the wt (wildtype) sequences (S176, S180), “pAA,” a primer specific for the mutated sequence (A176, A180), and “p776,” a primer for exon 7, followed by Eco47III digestion. FIG. 1, Panel C shows results of mRNA analysis by RT-PCR. Total liver RNA was converted to cDNA and amplified using primers p392 and p776 (top panel). Lane I was loaded with sample containing genomic DNA used as template, while lane 2 was loaded with sample containing Ikkα cDNA used as a template. PCR products were digested with Eco47III to reveal the presence of mutant sequence (bottom panel). FIG. 1, Panel D provides results from assays to determine IκB kinase activity in wt and mutant fibroblasts. Primary MEFs were left untreated or were stimulated for 15 min with mouse TNFα (20 ng/ml final concentration) or IL-1 (10 ng/ml final concentration). IKK complexes were isolated from cell lysates by immunoprecipitation with anti-IKKγ, and associated kinase activity was determined with GST-IκBα((1-54) as a substrate. FIG. 1, Panel E provides data showing the induction of NF-κB DNA binding activity in wt and mutant fibroblasts. Primary MEFs were stimulated with TNFα for the indicated times. The NF-κB and NF-1 DNA binding activities were then examined by electrophoretic mobility shift assay (EMSA).

FIG. 2 provides illustrations of the impaired lobuloalveolar development in Ikkα^{AA/AA} mammary glands. Panel A shows wholemount analyses, while Panel B shows histological analyses by H&E staining of wt and mutant mammary glands at different developmental stages. In this Figure, “V-6wk,” refers to virgin animals that were 6 weeks of age, “V-8wk,” refers to virgin animals that were 8 weeks of age, “P10,” refers to animals at day 10 of pregnancy, and “L1,” refers to animals at day 1 of lactation. Genotypes are indicated at the top. Panel C provides data showing the expression of milk protein genes, based on Northern analyses of total RNA extracted from wt and mutant mammary glands at different time points for expression of P-casein and whey acidic protein (WAP) mRNAs. CK18 is an epithelial specific marker. Results for two animals are shown for each genotype and time point.

FIG. 3 provides data showing impaired epithelial proliferation in Ikkα^{AA/AA} mammary glands. BrdU was administered at P10 or L1, and the mammary glands were removed 2 hours later. Paraffin sections of P10 glands were stained with anti-BrdU or anti-PCNA antibodies. Positive cells were visualized with Cyanine 3, while nuclei were counter-stained with DAPI. This Figure provides results for 300-400 epithelial cell nuclei examined per section of wt and Ikkα^{AA/AA} glands. The graph shows BrdU labeling indices, and the values provided represent the average fraction of BrdU-positive epithelial cells per total number of epithelial cells of 3 different mice. FIG. 4 provides data showing the defective cyclin DI induction in Ikkα^{AA/AA} mammary glands. Cyclin D1 immunostaining was performed on paraffin sections of P10 wt and mutant mammary glands using anti-cyclin D1 antibody. Positive cells were visualized with Cyanine 3, and nuclei were counter-stained with DAPI. The data presented here are from Western blot analyses of cyclin D1 expression in wt and Ikkα^{AA/AA} mammary glands. In this Figure, “V,” indicates virgin mice, “P6,” indicates day 6 of pregnancy, and “P10,” indicates day 10 of pregnancy. CK18 was used as an epithelial cell marker. The average relative expression levels are also indicated in the Figure, with wt given an arbitrary value of 1.00.

FIG. 5 provides data showing defective NF-κB activation in Ikkα^{AA/AA} mammary glands during pregnancy. Panel A provides data showing NF-κB DNA binding activity in wt and Ikkα^{AA} mammary glands examined using whole cell extracts and EMSA. NF-1 DNA binding activity was determined to control for extract quality and protein loading. Panel B provides the results of Western blot analysis of NF-κB proteins. In particular, whole cell extracts of wt and mutant mammary glands at P10 were analyzed by immunoblotting for expression of RelA (p65), RelB, c-Rel, NF-κB1 p105/p50, NF-κB2 p100/p52, IκBα, and IKKα.

FIG. 6 provides data showing defective NF-κB activation by RANKL in Ikkα^{AA/AA} mammary epithelial cells. Panel A provides data from Northern blot analyses of RANKL expression in wt and mutant mammary glands at different time points. Panel B provides data showing NF-κB activation in response to RANKL or TNFα. Primary cultures of wt or mutant mammary epithelial cells were stimulated with mTNFα (20 ng/ml) or mRANYL (100 ng/ml) for the indicated times. Nuclear extracts were prepared and the levels of NF-κB and NF-1 DNA binding activities were measured by EMSA.

FIG. 7 provides data showing the mammary specific expression of the IKB( superrepressor retards lobuloalveolar development. Panel A provides a schematic of the mammary specific superrepressor transgene with alanines instead of serines at positions 32 and 36. IκBα^{A32/36} was tagged with the HA epitope, and cloned downstream to the MMTV promoter. In this Figure, “Reg,” refers to the regulatory domain and “ANK,” refers to ankyrin repeats. Panel B provides data from PCR analysis of transgene DNA. GAPDH was used as a control. Panel C provides data from immunoblot analysis of transgene and cyclin D1 expression in wt and transgenic mammary glands. In this Figure, results are shown for extracts of L1 mammary glands immunoblotted with anti-HA and anti-cyclin D1 antibodies.

FIG. 8 provides data showing the rescue of the Ikkα^{AA/AA} mammary gland defect by a cyclin D1 transgene. Panel A provides data from immunoblot analysis of cyclin D1 expression in which protein extracts of L1 mammary glands were probed with anti-cyclin D1 and anti-actin antibodies. Panel B provides data showing NF-κB DNA binding activity. In these experiments, whole cell extracts of P10 mammary glands from wt, Ikkα^{AA/AA}, and Ikkα^{AA/AA} carrying the MMTV-cyclin D1 transgene were analyzed for NF-κB and NF-1 DNA binding activities. Panel C provides a schematic illustration of the signaling pathway that controls the proliferation of the mammary epithelium during pregnancy. The second arrow leading to cyclin D1 indicates additional signaling pathways that can regulate cyclin D1 expression.

FIG. 9 shows the proportion of breast tumor-free mice over a 36 month period of time in Ikkα^{+/+}/MMTV-c-neu females (filled squares) and Ikkα^{AA/AA}/MMTV-c-neu females (open squares).

FIG. 10 shows (A) the nucleotide sequence (SEQ ID NO:12) (GenBank # AF009225) and (B) encoded amino acid sequence (SEQ ID NO:13) (GenBank # AAC51671 and GenBank # AAC51671) of human IKKα.

FIG. 11 shows (A) the nucleotide sequence (SEQ ID NO:14) (NIH # NM-007700) and (B) encoded amino acid sequence (SEQ ID NO:15) (NIH # NP-031726) of mouse IKKα.

FIG. 12 shows (A) that homozygocity for the Ikkα^{AA} mutation reduces the incidence of mammary tumors caused by the MMTV-c-neu trans-oncogene but not the MMTV-v-Ha-Ras oncogene from observing breast cancer formation by biweekly palpation in Ikkα^{+/+}/MMTV-c-neu (or MMTV-v-Ha-Ras) and Ikkα^{AA/AA}/MMTV-c-neu (or MMTV-v-Ha-Ras) littermates with the Ikkα^{AA} mutation specific to Neu/ErbB2-induced tumors as it exerted no inhibitory effect on MMTV-v-Ha-Ras induced tumors and (B) shows that tumor number was markedly decreased in the Ikkα^{AA/AA}/MMTV-c-neu group, averaging 1.0 tumor per mouse vs 4.6 tumor per mouse for the wild type.

FIG. 13 shows (A) histology of tumors isolated from Ikkα^{AA/AA}/MMTV-c-Neu mice appears to be identical to tumors from the Ikkα^{AA}/MMTV-c-Neu mice with higher levels of NF-κB DNA binding activity in tumor tissue relative to the surrounding normal tissue with no significant differences in NF-κB DNA binding activity detected between tumors in the wt vs Ikkα^{AA/AA} backgrounds and (B) shows that Ikkα^{AA/AA}/MMTV-c-Neu cells exhibited less efficient induction of cyclin D1 in response to growth factor addback relative to Ikkα^{+/+}/MMTV-c-neu cells and that growth factor depletion and addback had no effect on cyclin D1 expression in Ha-Ras-induced tumor cells of either genotype.

FIG. 14 shows (A) an illustration of treatment of six weeks old wt and Ikkα^{AA/AA} mice given subcutaneous slow-release pellet of MPA (medroxyprogesterone acetate) followed by oral gavage of DMBA (7,12-dimethylbenzaanthracene) for effect of Ikkα^{AA} mutation on carcinogen-induced mammary cancer; where fifth generation mice of a backcross of Ikkα^{AA} to a BALB/c background was used for this experiment, B shows that Ikkα^{AA} mutation delays tumor onset where in the wt group, tumors started to appear after 5 weeks and 50% of mice had developed tumors by 7 weeks, while no tumors were detected before 10 weeks in Ikkα^{AA/AA} mice and it took 13.5 weeks for 50% of these mice to develop tumors and C shows that Ikkα^{AA} mutation reduces tumor incidence when mammary tumor formation was monitored twice a week after the last injection of DMBA (as in A) and C shows that tumor numbers were also decreased in the Ikkα^{AA/AA} mice, averaging 1.1 tumors per mouse vs 3.1 tumors per mouse for the wild type.

FIG. 15 shows that histologically, unlike the “signature” phenotype characterizing the ErbB2-inducing tumors, DMBA induced tumors display a variety of phenotypes whose spectrum is influenced by the Ikkα^{AA} mutation where the majority of the tumors derived from the wt mice (18-20 out of 30 examined by HE staining and microscopy) show sheets of undifferentiated tumor cells, while 75% of tumors derived from Ikkα^{AA/AA} mice (9 out 12 examined) show squamous metaplasia, with keratin swirls being the most prominent feature.

Definitions

To facilitate understanding of the invention, a number of terms are defined below.

The terms “sample” and “specimen” are used in their broadest sense and encompass samples or specimens obtained from any source, including buffer solutions, saline solutions, cell culture media, etc. As used herein, the term “biological samples” refers to samples or specimens obtained from animals (including humans, domestic animals, as well as feral or wild animals, such as ungulates, bear, fish, lagamorphs, rodents, etc.), and encompasses cells, fluids, solids, tissues, and gases. In preferred embodiments of this invention, biological samples include tissues (e.g., biopsy material), cell lines, cells isolated from tissue (whether or not the isolated cells are cultured after isolation from tissue), fixed cells (e.g., fixed for hisotological and/or immunohistochemical analysis), cerebrospinal fluid (CSF), serous fluid, blood, and blood products such as plasma, serum and the like. However, these examples are not to be construed as limiting the types of samples which find use with the present invention.

As used herein, the term “subject” refers to any animal, including humans.

As used herein, the term “animal” refers to any animal, preferably a mammal, and more preferably, the mammal includes, without limitation, human and non-human animals such simians, rodents, ovines, bovines, ruminants, lagomorphs, porcines, caprines, equines, canines, felines, aves, etc. Preferred non-human animals are members of the Order Rodentia (e.g., mouse and rat), sheep, pig, rabbit or cattle.

A “non-human animal” refers to any animal which is not a human and includes vertebrates such as rodents, non-human primates, ovines, bovines, ruminants, lagomorphs, porcines, caprines, equines, canines, felines, aves, etc. Preferred non-human animals are selected from the order Rodentia (particularly a mouse), sheep, pig, rabbit and cattle.

The “non-human animals having a genetically engineered genotype” of the invention are preferably produced by experimental manipulation of the genome of the germline of the non-human animal. These genetically engineered non-human animals may be produced by several methods including the introduction of a “transgene” comprising nucleic acid (usually DNA) into an embryonal target cell or integration into a chromosome of the somatic and/or germ line cells of a non-human animal by way of human intervention, such as by the methods described herein. “Transgenic non-human animals” are non-human animals whose genome has been manipulated by any molecular biological technique, including, for example, the introduction of a transgene, homologous recombination, knockin of a gene, and/or knockout of a gene.

The term “transgene” as used herein refers to a foreign gene that is placed into an organism by introducing the foreign gene into newly fertilized eggs or early embryos. The term “foreign gene” refers to any nucleic acid (e.g., gene sequence) which is introduced into the genome of an animal by experimental manipulations and may include gene sequences found in that animal so long as the introduced gene does not reside in the same location as does the naturally-occurring gene.

The terms “in operable combination,” “in operable order” and “operably linked” as used herein refer to the linkage of nicleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “treatment” or grammatical equivalents encompasses the improvement and/or reversal of the symptoms of disease (e.g., breast cancer). “Improvement in the physiologic function” in a non-human transgenic animals of the present invention may be assessed using any of the measurements described herein, as well as any effect upon the transgenic animals' survival. The response of treated transgenic animals and untreated transgenic animals is compared using any of the assays described herein (in addition, treated and untreated non-transgenic animals may be included as controls). A compound which causes an improvement in any parameter associated with disease when used in the screening methods of the instant invention may thereby be identified as a therapeutic compound.

As used herein, the term “at risk for disease” refers to a subject (e.g., a human) that is predisposed to experiencing a particular disease. This predisposition may be genetic (e.g., a particular genetic tendency to experience the disease, such as heritable disorders), or due to other factors (e.g., environmental conditions, exposures to detrimental compounds present in the environment, etc.). Thus, it is not intended that the present invention be limited to any particular risk, nor is it intended that the present invention be limited to any particular disease.

As used herein, the term “suffering from disease” refers to a subject (e.g., a human) that is experiencing a particular disease. It is not intended that the present invention be limited to any particular signs or symptoms, nor disease. Thus, it is intended that the present invention encompass subjects that are experiencing any range of disease, from sub-clinical infection to full-blown disease, wherein the subject exhibits at least some of the indicia (e.g., signs and symptoms) associated with the particular disease.

As used herein, the terms “disease” and “pathological condition” are used interchangeably to a state, signs, and/or symptoms that are associated with any impairment of the normal state of a living animal or of any of its organs or tissues that interrupts or modifies the performance of normal functions, and may be a response to environmental factors (such as malnutrition, industrial hazards, or climate), to specific infective agents (such as worms, bacteria, or viruses), to inherent defect of the organism (such as various genetic anomalies, or to combinations of these and other factors.

The term “compound” refers to any chemical entity, pharmaceutical, drug, and the like that can be used to treat or prevent a disease, illness, sickness, or disorder of bodily function. Compounds comprise both known and potential therapeutic compounds. A compound can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of disease (e.g., cancer).

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

A “transformed cell” is a cell or cell line that has acquired the ability to grow in cell culture for many multiple generations, the ability to grow in soft agar and the ability to not have cell growth inhibited by cell-to-cell contact. In this regard, transformation refers to the introduction of foreign genetic material into a cell or organism. Transformation may be accomplished by any method known which permits the successful introduction of nucleic acids into cells and which results in the expression of the introduced nucleic acid. “Transformation” includes but is not limited to such methods as transfection, microinjection, electroporation, and lipofection (liposome-mediated gene transfer). Transformation may be accomplished through use of any expression vector. For example, the use of baculovirus to introduce foreign nucleic acid into insect cells is contemplated. The term “transformation” also includes methods such as P-element mediated germline transformation of whole insects. Additionally, transformation refers to cells that have been transformed naturally, usually through genetic mutation.

As used herein, the term “kit” is used in reference to a combination of reagents and other materials. It is contemplated that the kit may include reagents such as antibodies, control proteins, as well as testing containers (e.g., microtiter plates, etc.). It is not intended that the term “kit” be limited to a particular combination of reagents and/or other materials.

As used herein, the term “purified” or “to purify” or “purification” refers to the removal or reduction of at least one contaminant from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample (i.e., “enrichment” of an antibody).

The terms “Western blot,” “Western immunoblot,” “immunoblot,” and “Western” refer to the immunological analysis of protein(s), polypeptides or peptides that have been immobilized onto a membrane support. The proteins are first resolved by acrylamide gel electrophoresis to separate the proteins, followed by transfer of the protein from the gel to a solid support, such as nitrocellulose or a nylon membrane. The immobilized proteins are then exposed to an antibody having reactivity towards an antigen of interest. The binding of the antibody (i.e., the primary antibody) is detected by use of a secondary antibody which specifically binds the primary antibody. The secondary antibody is typically conjugated to an enzyme which permits visualization by the production of a colored reaction product or catalyzes a luminescent enzymatic reaction (e.g., ECL reagent, Amersham).

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size, followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (Sambrook, J. et al., supra, pp 7.39-7.52 [1989]).

As used herein, the term “ELISA” refers to enzyme-linked immunosorbent assay. Numerous methods and applications for carrying out an ELISA are well known in the art, and provided in many sources (See, e.g., Crowther, “Enzyme-Linked Immunosorbent Assay (ELISA),” in Molecular Biomethods Handbook, Rapley et al. [eds.], pp. 595-617, Humana Press, Inc., Totowa, N.J. [1998]; Harlow and Lane (eds.), Antibodies: A Laboratory Manual, Cold Spring Harbor Laboratory Press [1988]; and Ausubel et al. (eds.), Current Protocols in Molecular Biology, Ch. 11, John Wiley & Sons, Inc., New York [1994]).

In one embodiment of the present invention, a “direct ELISA” protocol is provided, where an antigen is first bound and immobilized to a microtiter plate well. In an alternative embodiment, a “sandwich ELISA” is provided, where the antigen is attached to the stationary phase by capturing it with an antibody that has been previously bound to the microtiter plate well. The ELISA method detects an immobilized antigen by use of an antibody-enzyme conjugate, where the antibody is specific for the antigen of interest, and the enzyme portion allows visualization and quantitation by the generation of a colored or fluorescent reaction product. The conjugated enzymes commonly used in the ELISA include horseradish peroxidase, urease, alkaline phosphatase, glucoamylase or O-galactosidase. The intensity of color development is proportional to the amount of antigen present in the reaction well.

As used herein, the term “in vitro” refers to an artificial environment and to processes or reactions that occur within an artificial environment. In vitro environments consist of, but are not limited to, controlled laboratory conditions. The term “in vivo” refers to the natural environment (e.g., within an organism or a cell) and to processes or reactions that occur within that natural environment.

As used herein, the term “wild-type” refers to a gene or gene product which has the characteristics of that gene or gene product when isolated from a naturally occurring source. A wild-type gene is that which is most frequently observed in a population and is thus arbitrarily designated as the “normal” or “wild-type” form of the gene. In contrast, the term “modified” (or “mutant”) refers to a gene or gene product which displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

“Nucleic acid sequence,” “nucleotide sequence,” and “polynucleotide sequence” as used herein refer to an oligonucleotide or polynucleotide, and fragments or portions thereof, and to DNA or RNA of genomic or synthetic origin which may be single- or double-stranded, and represent the sense or antisense strand.

As used herein, the terms “oligonucleotides” and “oligomers” refer to a nucleic acid sequence of at least about 10 nucleotides and as many as about 60 nucleotides, preferably about 15 to 30 nucleotides, and more preferably about 20-25 nucleotides, which can be used as a probe or amplimer.

The term “nucleotide sequence of interest” refers to any nucleotide sequence, the manipulation of which may be deemed desirable for any reason, by one of ordinary skill in the art. Such nucleotide sequences include, but are not limited to, coding sequences of structural genes (e.g., reporter genes, selection marker genes, oncogenes, drug resistance genes, growth factors, etc.), and of non-coding regulatory sequences that do not encode an mRNA or protein product (e.g., promoter sequence, enhancer sequence, polyadenylation sequence, termination sequence, etc.).

“Amino acid sequence,” “polypeptide sequence,” “peptide sequence,” and “peptide” are used interchangeably herein to refer to a sequence of amino acids.

As used herein, the terms “peptide,” “polypeptide” and “protein” all refer to a primary sequence of amino acids that are joined by covalent “peptide linkages.” In general, a peptide consists of a few amino acids, typically from 2-50 amino acids, and is shorter than a protein. The term “polypeptide” may encompass either peptides or proteins. A peptide, polypeptide or protein may be synthetic, recombinant or naturally occurring. A synthetic peptide is a peptide which is produced by artificial means in vitro (e.g., was not produced in vivo).

The term “portion” when used in reference to an amino acid sequence and nucleotide sequence refers to fragments of that amino acid sequence and nucleotide sequence, respectively. Fragments of nucleotide sequences may range in size from 5, preferably from 10, nucleotide residues to the entire nucleotide sequence minus one nucleic acid residue. Thus, a nucleic acid sequence comprising “at least a portion of” a nucleotide sequence comprises from five (5) contiguous nucleotide residues of the nucleotide sequence to the entire nucleotide sequence. Fragments of an amino acid sequence may range in size from 3, preferably 5, amino acids to the entire amino acid sequence minus one amino acid residue. Thus, a polypeptide sequence comprising “at least a portion of” an amino acid sequence comprises from three (3) contiguous amino acid residues of the amino acid sequence to the entire amino acid sequence.

An oligonucleotide sequence which is a “homolog” of a first nucleotide sequence is defined herein as an oligonucleotide sequence which exhibits greater than or equal to 50% identity, and more preferably greater than or equal to 70% identity, to the first nucleotide sequence when sequences having a length of 10 bp or larger are compared.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects that transcription proceeds in a 5′ to 3′ direction along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

The term “cloning” as used herein, refers to the process of isolating a nucleotide sequence from a nucleotide library, cell or organism for replication by recombinant techniques.

The term “recombinant DNA molecule” as used herein refers to a DNA molecule which is comprised of segments of DNA joined together by means of molecular biological techniques.

The term “recombinant protein” or “recombinant polypeptide” as used herein refers to a protein molecule which is expressed using a recombinant DNA molecule.

The term “transfection” as used herein refers to the introduction of foreign DNA into cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, biolistics (i.e., particle bombardment) and the like.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “5′-CAGT-3′,” is complementary to the sequence “5′-ACTG-3′.” Complementarity can be “partial” or “total.” “Partial” complementarity is where one or more nucleic acid bases is not matched according to the base pairing rules, for example from 51% to 99%, preferably from 70% to 99%, more preferably from 80% to 99%, and most preferably from 90% to 99%, matched. “Total” or “complete” complementarity between nucleic acids is where each and every nucleic acid base is matched with another base under the base pairing rules (i.e., 100% matched). The degree of complementarity between nucleic acid strands may have significant effects on the efficiency and strength of hybridization between nucleic acid strands. This may be of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary (i.e., “substantially homologous”) to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

As used herein the term “stringency” is used in reference to the conditions of temperature, ionic strength, and the presence of other compounds such as organic solvents, under which nucleic acid hybridizations are conducted. “Stringency” typically occurs in a range from about Tₘ° C. to about 20° C. to 25° C. below Tₘ. As will be understood by those of skill in the art, a stringent hybridization can be used to identify or detect identical polynucleotide sequences or to identify or detect similar or related polynucleotide sequences. Under “stringent conditions” the nucleotide sequence portions thereof, will hybridize to its exact complement and closely related sequences.

Low stringency conditions comprise conditions equivalent to binding or hybridization at 68° C. in a solution consisting of 5× SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent (50× Denhardt's contains per 500 ml: 5 g Ficoll (Type 400, Pharmacia), 5 g BSA (Fraction V; Sigma)) and 100,g/ml denatured salmon sperm DNA followed by washing in a solution comprising 2.0× SSPE, 0.1% SDS at room temperature when a probe of about 100 to about 1000 nucleotides in length is employed.

High stringency conditions when used in reference to nucleic acid hybridization comprise conditions equivalent to binding or hybridization at 68° C. in a solution consisting of 5× SSPE, 1% SDS, 5× Denhardt's reagent and 100 μg/ml denatured salmon sperm DNA followed by washing in a solution comprising 0.1× SSPE, and 0.1% SDS at 68° C. when a probe of about 100 to about 1000 nucleotides in length is employed.

It is well known in the art that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which promote hybridization under conditions of high stringency (e.g., increasing the temperature of the hybridization and/or wash steps, the use of formamide in the hybridization solution, etc.) are well known in the art.

The term “equivalent” when made in reference to a hybridization condition as it relates to a hybridization condition of interest means that the hybridization condition and the hybridization condition of interest result in hybridization of nucleic acid sequences which have the same range of percent (%) homology. For example, if a hybridization condition of interest results in hybridization of a first nucleic acid sequence with other nucleic acid sequences that have from 50% to 70% homology to the first nucleic acid sequence, then another hybridization condition is said to be equivalent to the hybridization condition of interest if this other hybridization condition also results in hybridization of the first nucleic acid sequence with the other nucleic acid sequences that have from 50% to 70% homology to the first nucleic acid sequence.

When used in reference to a double-stranded nucleic acid sequence such as a cDNA or genomic clone, the term “substantially homologous” refers to any probe which can hybridize either partially or completely to either or both strands of the double-stranded nucleic acid sequence under conditions of low stringency as described above.

When used in reference to a single-stranded nucleic acid sequence, the term “substantially homologous” refers to any probe which can hybridize (i.e., it is the complement of) the single-stranded nucleic acid sequence under conditions of low stringency as described above.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀t or R₀t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ81.5+0.41(% G+C), when a nucleic acid is in aqueous solution at 1 M NaCl (see e.g., Anderson and Young, Quantitative Filter Hybridization, in Nucleic Acid Hybridization [1985]). Other references include more sophisticated computations which take structural as well as sequence characteristics into account for the calculation of Tₘ.

The term “heterologous nucleic acid sequence” or “heterologous DNA” are used interchangeably to refer to a nucleotide sequence which is ligated to a nucleic acid sequence to which it is not ligated in nature, or to which it is ligated at a different location in nature. Heterologous DNA is not endogenous to the cell into which it is introduced, but has been obtained from another cell. Generally, although not necessarily, such heterologous DNA encodes RNA and proteins that are not normally produced by the cell into which it is expressed. Examples of heterologous DNA include reporter genes, transcriptional and translational regulatory sequences, selectable marker proteins (e.g., proteins which confer drug resistance), etc.

“Amplification” is defined herein as the production of additional copies of a nucleic acid sequence and is generally carried out using polymerase chain reaction technologies well known in the art (see, e.g., Dieffenbach and Dveksler, PCR Primer, a Laboratory Manual, Cold Spring Harbor Press, Plainview N.Y. [1995]). As used herein, the term “polymerase chain reaction” (“PCR”) refers to the methods of U.S. Pat. Nos. 4,683,195, 4,683,202, and 4,965,188, all of which are hereby incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

The terms “reverse transcription polymerase chain reaction” and “RT-PCR” refer to a method for reverse transcription of an RNA sequence to generate a mixture of cDNA sequences, followed by increasing the concentration of a desired segment of the transcribed cDNA sequences in the mixture without cloning or purification. Typically, RNA is reverse transcribed using a single primer (e.g., an oligo-dT primer) prior to PCR amplification of the desired segment of the transcribed DNA using two primers.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and of an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that it is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “restriction endonucleases” and “restriction enzymes” refer to bacterial enzymes, each of which cut double- or single-stranded DNA at or near a specific nucleotide sequence.

As used herein, the term “an oligonucleotide having a nucleotide sequence encoding a gene” means a nucleic acid sequence comprising the coding region of a gene (i.e. the nucleic acid sequence which encodes a gene product). The coding region may be present in either cDNA, genomic DNA or RNA form. When present in a DNA form, the oligonucleotide may be single-stranded (i.e., the sense strand) or double-stranded. Suitable control elements such as enhancers, promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers, splice junctions, intervening sequences, polyadenylation signals, etc. or a combination of both endogenous and exogenous control elements.

Transcriptional control signals in eukaryotes comprise “enhancer” elements. Enhancers consist of short arrays of DNA sequences that interact specifically with cellular proteins involved in transcription (Maniatis et al., Science 236:1237 [1987]). Enhancer elements have been isolated from a variety of eukaryotic sources including genes in plant, yeast, insect and mammalian cells and viruses. The selection of a particular enhancer depends on what cell type is to be used to express the protein of interest.

The presence of “splicing signals” on an expression vector often results in higher levels of expression of the recombinant transcript. Splicing signals mediate the removal of introns from the primary RNA transcript and consist of a splice donor and acceptor site (Sambrook, et al. Molecular Cloning: A Laboratory Manual, 2nd ed., Cold Spring Harbor Laboratory Press, New York, pp. 16.7-16.8 [1989]). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is isolated from one gene and placed 3′ of another gene.

The term “promoter,” “promoter element,” or “promoter sequence” as used herein, refers to a DNA sequence which when placed at the 5′ end of (i.e., precedes) an oligonucleotide sequence is capable of controlling the transcription of the oligonucleotide sequence into mRNA. A promoter is typically located 5′ (i.e., upstream) of an oligonucleotide sequence whose transcription into mRNA it controls, and provides a site for specific binding by RNA polymerase and for initiation of transcription.

The term “promoter activity” when made in reference to a nucleic acid sequence refers to the ability of the nucleic acid sequence to initiate transcription of an oligonucleotide sequence into mRNA.

As used herein, the terms “nucleic acid molecule encoding,” “nucleotide encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “isolated” when used in relation to a nucleic acid, as in “an isolated oligonucleotide” refers to a nucleic acid sequence that is separated from at least one contaminant nucleic acid with which it is ordinarily associated in its natural source. Isolated nucleic acid is nucleic acid present in a form or setting that is different from that in which it is found in nature. In contrast, non-isolated nucleic acids are nucleic acids such as DNA and RNA which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs which encode a multitude of proteins. However, isolated nucleic acid encoding a polypeptide of interest includes, by way of example, such nucleic acid in cells ordinarily expressing the polypeptide of interest where the nucleic acid is in a chromosomal or extrachromosomal location different from that of natural cells, or is otherwise flanked by a different nucleic acid sequence than that found in nature. The isolated nucleic acid or oligonucleotide may be present in single-stranded or double-stranded form. Isolated nucleic acid can be readily identified (if desired) by a variety of techniques (e.g., hybridization, dot blotting, etc.). When an isolated nucleic acid or oligonucleotide is to be utilized to express a protein, the oligonucleotide will contain at a minimum the sense or coding strand (i.e., the oligonucleotide may be single-stranded). Alternatively, it may contain both the sense and anti-sense strands (i.e., the oligonucleotide may be double-stranded).

As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

As used herein, the term “structural gene” or “structural nucleotide sequence” refers to a DNA sequence coding for RNA or a protein which does not control the expression of other genes. In contrast, a “regulatory gene” or “regulatory sequence” is a structural gene which encodes products (e.g., transcription factors) which control the expression of other genes.

As used herein, the term “regulatory element” refers to a genetic element which controls some aspect of the expression of nucleic acid sequences. For example, a promoter is a regulatory element which facilitates the initiation of transcription of an operably linked coding region. Other regulatory elements include splicing signals, polyadenylation signals, termination signals, etc.

As used herein, the term “gene” means the deoxyribonucleotide sequences comprising the coding region of a structural gene. A “gene” may also include non-translated sequences located adjacent to the coding region on both the 5′ and 3′ ends such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogenous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, post-transcriptional cleavage and polyadenylation.

As used herein, the terms “auto-antibody” or “auto-antibodies” refer to any immunoglobulin that binds specifically to an antigen that is native to the host organism that produced the antibody (i.e., the antigen is not synthetic and/or has not been artificially supplied to the host organism). However, the term encompasses antibodies originally produced in response to the administration or presence of a foreign and/or synthetic substance in the host, but also cross-react with “self” antigens. The presence of auto-antibodies is termed “autoimmunity.”

As used herein, the term “antigen” is used in reference to any substance that is capable of reacting with an antibody. It is intended that this term encompass any antigen and “immunogen” (i.e., a substance which induces the formation of antibodies). Thus, in an immunogenic reaction, antibodies are produced in response to the presence of an antigen or portion of an antigen.

The term “antigenic determinant” or “epitope” as used herein refers to that portion of an antigen that makes contact with a particular antibody variable region. When a protein or fragment of a protein is used to immunize a host animal, numerous regions of the protein may induce the production of antibodies which bind specifically to a given region or three-dimensional structure on the protein; these regions or structures are referred to as antigenic determinants.

A “cultured cell” is a cell which has been maintained and/or propagated in vitro. Cultured cells include primary cultured cells and cell lines.

“Primary cultured cells” are primary cells which are in in vitro culture and which preferably, though not necessarily, are capable of undergoing ten or fewer passages in in vitro culture before senescence and/or cessation of proliferation.

The terms “cell line” and “immortalized cell” refer to a cell which is capable of a greater number of cell divisions in vitro before cessation of proliferation and/or senescence as compared to a primary cell from the same source. A cell line includes, but does not require, that the cells be capable of an infinite number of cell divisions in culture. Cell lines include, but are not limited to, finite cell lines and continuous cell lines. As used herein, the term “finite cell line” refers to a cell line which is capable of a limited number (from about 1 to about 50, more preferably from about 1 to about 40, and most preferably from about 1 to about 20) of cell divisions prior to senescence.

The terms “neoplasm” and “tumor” refer to a tissue growth. Often, though not necessarily, neoplasms are characterized, in part, by angiogenesis. Neoplasms may be benign and are exemplified, but not limited to, a hemangioma, glioma, teratoma, and the like. Neoplasms may alternatively be malignant, for example, a carcinoma, sarcoma, glioblastoma, astrocytoma, neuroblastoma, retinoblastoma, and the like.

The terms “malignant neoplasm” and “malignant tumor” refer to a neoplasm which contains at least one cancer cell. A “cancer cell” refers to a cell undergoing early, intermediate or advanced stages of multi-step neoplastic progression as previously described [H.C. Pitot (1978) in “Fundamentals of Oncology,” Marcel Dekker (Ed.), New York pp 15-28]. The features of early, intermediate and advanced stages of neoplastic progression have been described using microscopy. Cancer cells at each of the three stages of neoplastic progression generally have abnormal karyotypes, including translocations, inversion, deletions, isochromosomes, monosomies, and extra chromosomes. A cell in the early stages of malignant progression is referred to as “hyperplastic cell” and is characterized by dividing without control and/or at a greater rate than a normal cell of the same cell type in the same tissue. Proliferation may be slow or rapid but continues unabated. A cell in the intermediate stages of neoplastic progression is referred to as a “dysplastic cell.” A dysplastic cell resembles an immature epithelial cell, is generally spatially disorganized within the tissue and loses its specialized structures and functions. During the intermediate stages of neoplastic progression, an increasing percentage of the epithelium becomes composed of dysplastic cells. “Hyperplastic” and “dysplastic” cells are referred to as “pre-neoplastic” cells. In the advanced stages of neoplastic progression a dysplastic cell become a “neoplastic” cell. Neoplastic cells are typically invasive i.e., they either invade adjacent tissues, or are shed from the primary site and circulate through the blood and lymph to other locations in the body where they initiate one or more secondary cancers, i.e., “metastases.” Thus, the term “cancer” is used herein to refer to a malignant neoplasm, which may or may not be metastatic.

The term “cancer cell” refers to a cell undergoing early, intermediate or advanced stages of multi-step neoplastic progression as previously described [H. C. Pitot (1978) in “Fundamentals of Oncology,” marcel Dekker (Ed.), New York pp 15-28]. The features of early, intermediate and advanced stages of neoplastic progression have been described using microscopy. Cancer cells at each of the three stages of neoplastic progression generally have abnormal karyotypes, including translocations, inversion, deletions, isochromosomes, monosomies, and extra chromosomes. A cell in the early stages of malignant progression is referred to as “hyperplastic cell” and is characterized by dividing without control and/or at a greater rate than a normal cell of the same cell type in the same tissue. Proliferation may be slow or rapid but continues unabated. A cell in the intermediate stages of neoplastic progression is referred to as a “dysplastic cell.” A dysplastic cell resembles an immature epithelial cell, is generally spatially disorganized within the tissue and loses its specialized structures and functions. During the intermediate stages of neoplastic progression, an increasing percentage of the epithelium becomes composed of dysplastic cells. “Hyperplastic” and “dysplastic” cells are referred to as “pre-neoplastic” cells. In the advanced stages of neoplastic progression a dysplastic cell become a “neoplastic” cell. Neoplastic cells are typically invasive i.e., they either invade adjacent tissues, or are shed from the primary site and circulate through the blood and lymph to other locations in the body where they initiate secondary cancers. The term “cancer” or “neoplasia” refers to a plurality of cancer cells.

The term “epithelial cell” refers to a cuboidal-shaped, nucleated cell which generally located on the surface of a tissue. A layer of epithelial cells generally functions to provide a protective lining and/or surface that may also be involved in transport processes. An epithelial cell is readily distinguished from a non-epithelial cell (e.g., muscle cell, nerve cell, etc.) using histological methods well known in the art.

The term “control” as used herein when in reference to a sample, cell, tissue, animal, etc., refers to any type of sample, cell, tissue, animal, etc. that one of ordinary skill in the art may use for checking the results of another sample, cell, tissue, animal, etc., by maintaining the same conditions except in some one particular factor, and thus inferring the causal significance of this varied factor.

### DESCRIPTION OF THE INVENTION

The present invention provides compositions and methods for the suppression of mammary epithelial cell proliferation. In particular, the present invention provides compositions and methods for IKKα kinase inhibition in breast cancer therapy that is relatively free of toxic side effects.

The present invention was based, in part, on the inventors' discovery that reducing IKKα kinase activity results in a reduction in mammary epithelial cell proliferation. Based on this, the inventors further discovered that inhibitors of IKKα kinase activity are useful in breast cancer therapy since such inhibitors reduce tumor mammary cell proliferation that is associated with tumor progression. Additionally, the inventors discovered that agents that are useful in breast tumor therapy may be screened by determining whether such agents act as inhibitors of IKKα kinase activity. Further still, it is the inventors' view that tumor mammary epithelial cells may be detected by screening tissues for elevated levels of IKKα kinase activity.

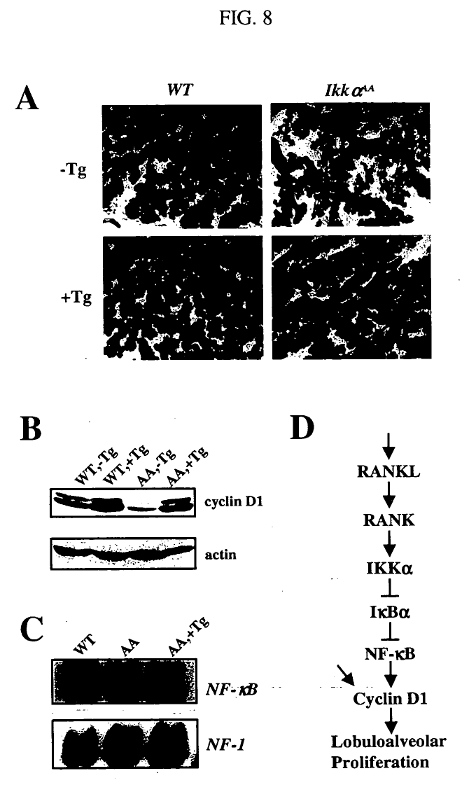

In particular, the inventors discovered that transgenic Ikkα^{AA/AA} female mice that contain a knockin Ikkα^{AA} allele that inactivates the kinase activity of IKKα are characterized by defective proliferation of the lobuloalveolar tree during pregnancy and lactation. The inventors also discovered that IKKα kinase activity was required for NF-κB activation in the mammary epithelium during pregnancy and in response to RANKL application as well as for cyclin D1 induction, and that IKKα kinase activity was not required for TNFα responsiveness. These discoveries further led the inventors to determine that IKKα kinase activity plays a central role in the proliferation of mammary epithelium (FIG. 8, panel C).

One advantage of the inventions' compositions and methods is that they provide therapeutic approaches that have low toxic side effects. This is based on the inventors' observation that while inhibiting of IKKα kinase activity reduced mammary epithelial cell proliferation, this inhibition did not adversely impact the treated subject's immune response, the development of other epithelial cells (e.g., epidermal cells), or limb development.

### DETAILED DESCRIPTION OF THE INVENTION

The present invention provides compositions and methods for the suppression of mammary epithelial cell proliferation. In particular, the present invention provides compositions and methods for IKKα kinase inhibition in breast cancer therapy that is relatively free of toxic side effects.

In the present invention, IKKα was found to be required for NF-κB activation in the mammary epithelium during pregnancy and in response to RANKL application. IKKα activity, however, is not required for TNFα responsiveness. Furthermore, IKKα activity was found to be required for induction of cyclin D1. Interestingly, cyclin D1 was previously suggested to be an NF-κB target gene (Guttridge et al., Mol. Cell Biol., 19:5785-5799 [1999]) and its ablation results in a mammary gland defect similar to the one displayed by Rank^{-/-}, Rankl^{-/-} and Ikkα^{AA/AA} mice (See e.g., Fantl et al., Genes Dev., 9:2364-2372 [1995]; and Sicinski et al., Cell 82:621-630 [1995]).

In the development of the present invention, a “knockin” Ikkα^{AA} allele was generated in which the two codons specifying the activation loop serines, whose phosphorylation is required for kinase activation, were replaced with alanine codons. Data provided herein demonstrates that inhibition of NF-κB in the mammary gland achieved by tissue specific expression of a non-phosphorylatable IκBα mutant (Iκbα^{A32/36}) was found also to result in decreased cyclin D1 expression and defective lobuloalveolar proliferation. Conversely, a mammary gland-specific cyclin D1 transgene was found to completely rescue the lactation defect in Ikkα^{AA/AA} mice. These results establish IKKα as the critical intermediate in a signaling pathway initiated by binding of RANKL to RANK, that leads to NF-κB-dependent cyclin D1 induction in mammary epithelial cells. This pathway is responsible for proliferation of lobuloalveolar cells during pregnancy. As elevated cyclin D1 is required for breast carcinogenesis (Yu et al., Nature 411:2162-2167 [2001]) and Ikkα^{AA/AA} mice do not exhibit the severe sensitivity to apoptosis and infections that is displayed by IKKβ-deficient mice (Li et al., Science 284:321-325 [1999]; Li et al., J. Exp. Med., 189:1839-1845 [1999]; and Senftleben et al., [2001a], supra), data obtained during the development of the present invention demonstrate that IKKα kinase inhibition is suitable for use in breast cancer therapy that is relatively free of toxic side effects.

The invention is further is described under (A) Mammary Gland Development, (B) Generation of The Exemplary Ikkα^{AA/AA} Knockin Mice, (C) IKKα is Essential for Lactation and Lobuloalveolar Development During Pregnancy As Demonstrated By the Transgenic Ikkα^{AA/AA} Animals Of The Invention, (D) The Lactation Defect in The Exemplary Ikkα^{AA/AA} Mice is Due to Impaired Epithelial Cell Proliferation, (E) Defective Cyclin D1 Induction in Ikkα^{AA/AA} Mammary Glands Of the Invention's Transgenic Mice, (F) Defective NF-κB Activation in The Exemplary Ikkα^{AA/AA} Mammary Glands Of The Invention's Transgenic Mice, (G) RANKL Activates NF-κB in Wild-Type, But Not Ikkα^{AA/AA} Mammary Epithelial Cells Of The Invention's Transgenic Mice, (H) Inhibition of NF-κB Activity in Mammary Glands of Transgenic Mice Of The Invention Inhibits Their Development, (I) The Ikkα^{AA/AA} Mutant Phenotype Of The Invention's Transgenic Mice is Rescued by a Cyclin D1 Transgene, (J) RANK, NF-κB and the Control of Mammary Gland Development, (K) The Many Functions of IKKα, (L) Reducing Mammary Epithelial Cell Proliferation, (M) Reducing Mammary Epithelial Cell Proliferation And Mammary Tumors In A Subject, (N) Detecting Tumor Mammary Epithelial Cells, (O) Screening Compounds, and (P) Transgenic Animals.

A. Mammary Gland Development

NF-κB transcription factors are well-established as critical regulators of innate and adaptive immune responses (Delhase and Karin, Cold Spring Harbor Symp. Quant. Biol., 64:491-503 [1999]; and Ghosh et al., Ann. Rev. Immunol., 16:225-260 [1998]). By inducing genes coding for cytokines, chemokines and adhesion molecules, NF-κB is also thought to be a key player in chronic inflammatory diseases (Barnes and Karin, New Eng. J. Med., 336:1066-1071 [1997]). Substantial evidence suggests that NF-κB may also be involved in carcinogenesis (Gilmore et al., “Misregulation of a signal transduction pathway: The role of Rel/NF-κB transcription factors in oncogenesis,” in Ehrlich (ed.), “DNA Alterations in Cancer,” Bio Techniques Books, Natick, MA [1999]). Activation of NF-κB inhibits apoptosis by tumor necrosis factor (TNF)α (Beg and Baltimore, Science 274:782-784 [1996]; Liu et al., Cell 87:565-576 [1996]; and Van Antwerp et al., Science 274:787-789 [1996]) and various anti-cancer drugs and radiation (Wang et al., Nature 369:669-671 [1996]). In addition, NF-κB activation may promote cell proliferation (Bargou et al., J. Clin. Invest., 100:2961-2969 [1997]; Guttridge et al., Mol. Cell. Biol., 19:5785-5799 [1999]; Hinz et al., Mol. Cell Biol., 19:2690-2698 [1999]; Kontgen et al., Genes Dev., 9:1965-1977 [1995]; and Snapper et al., J. Immunol., 156:183-191 [1996]).

The c-rel gene, encoding a component of NF-κB, is the cellular homolog of the v-rel oncogene (Gilmore, Oncogene 18:6925-6937 [1999]) and chromosomal translocations that constitutively activate NF-κB2 (another NF-κB component) have been linked to lymphomas (Gilmore et al., supra; and Neri et al., Cell 67:1075-1087 [1991]). Elevated NF-κB DNA binding activity was detected in a variety of lymphoid and non-lymphoid tumors (Gilmore et al., supra). In particular, constitutive NF-κB DNA binding activity was found in a majority of hormone-dependent and -independent human breast cancers as well as chemically-induced breast cancers in rodents (Cogswell et al., Oncogene 19:1123-1131 [2000]; Nakshatri et al., Mol. Cell Biol., 17:3629-3639 [1997]; and Sovak et al., J. Clin. Invest., 100:2952-2960 [1997]). However, these studies have not clearly indicated whether the elevated level of NF-κB is just a marker of breast tumor cells or whether it plays a causative role in tumor development by suppressing apoptosis or promoting cell proliferation. This is of particular importance as NF-κB activity is also induced during normal mammary gland development (Clarkson et al., J. Biol. Chem., 275:12737-12742 [2000]; and Geymayer and Doppler, FASEB J., 14:1159-1170 [2000]).

The mammary gland is an ideal system for studying the control of organogenesis. As described herein, the mammary gland and its development were investigated during the development of the present invention to determine the potential role of NF-κB in this process. Mammary gland development can be divided into seven stages (Hennighausen and Robinson, Genes Dev., 12:449-455 [1998]; and Medina, J. Mammary Gland Biol. Neoplasia 1:5-19 [1996]). In the mouse, the embryonic stage starts at E11 with small epithelial buds that increase in size until E15.5, when they begin to invade the fat pad that forms the surrounding mesenchyme. At birth, the mammary epithelium has formed distinct ductal and lobular units. During the postnatal stage (days 1-21), the ducts elongate and start branching. Ductal growth increases during the juvenile stage, which starts at week 3 postnatally. Terminal end buds with high mitotic rates begin to form and invade the stromal fat pad. High rates of apoptosis can be detected in the interior of the end bud. During puberty, the mitotic rate remains high while the rate of apoptosis declines, resulting in net ductal growth. Most tissue growth, however, occurs during pregnancy, involving extensive and rapid proliferation of the mammary epithelium, as well as extensive branching of the ductal tree. At this point, the gland is composed mostly of lobuloalveolar epithelium. The next stage, lactation, begins near the end of pregnancy; during this stage, epithelial cells undergo terminal differentiation to synthesize milk proteins. The last stage, involution, begins at weaning; during this stage, the entire ductal tree regresses in size, through apoptosis, to that of the pubescent gland. NF-κB activation occurs in two waves during this process, namely during pregnancy (peaking around days 15-16 post-coitum), and during involution (Clarkson et al., supra; and Geymayer and Doppler, supra). The exact role of NF-κB in mammary gland development is not clear; it has been suggested to either maintain the proliferative state of lobuloalveolar cells, prevent their terminal differentiation, or delay the onset of apoptosis during involution.

Not only is the function of NF-κB in mammary gland development obscure, but also the signaling pathway responsible for its activation during this organogenic process is not known. Most commonly, NF-κB activity is regulated through interaction of NF-κB dimers with specific inhibitors, the IκBs, which mask their NF-κB nuclear localization signal and trap them in the cytoplasm (Ghosh et al., supra). In response to cell stimulation with proinflammatory and innate immune stimuli (e.g., TNFα, interleukin 1 (IL-1) or bacterial endotoxin), the IκBs are phosphorylated at two conserved serines, a modification that targets them to rapid ubiquitin-dependent proteolysis (Karin and Ben-Neriah, Ann. Rev. Immunol., 18:621-663 [2000]). IKB phosphorylation is carried out by the IκB kinase (IKK), a protein complex composed of three subunits: IKKα, IKKβ and IKKγ/NEMO (Rothwarf and Karin, “The NF-κB activation pathway: A paradigm in information transfer from membrane to nucleus,” Science's STKE www.stke.org/cgi/content/fullO_sigtrans; 1999/5/rel [1999]).

IKKα and IKKβ are 52% identical in sequence and serve as the catalytic subunits of the complex, whereas IKKγ/NEMO is the regulatory subunit. IKKα and IKKβ contain very similar kinase domains with essentially identical activation loops (Mercurio et al., Science 278:860-866 [1997]; Woronicz et al., Science 278:866-869 [1997]; and Zandi et al., Cell 91:243-252 [1997]). Despite their structural and biochemical similarities, IKKα and IKKβ are functionally distinct (Rothwarf and Karin, supra). Whereas IKKβ is essential for NF-κB activation in response to proinflammatory and innate immune stimuli, IKKα is not required for such responses (Chu et al., Immun., 11:721-731 [1999]; Hu et al., Science 284:316-320 [1999]; Li et al., Science 284:321-325 [1999]; Li et al., J. Exp. Med., 189:1839-1845 [1999]; Senftleben et al., Immun., 14:217-230 [2001a]; and Tanaka et al., Immun., 10:421-429 [1999]). However, IKKα plays a unique and critical role in development of the epidermis (Hu et al., stupra; and Takeda et al., Science 284:313-316 [1999]), but its ability to induce keratinocyte differentiation is independent of its protein kinase activity or NF-κB activation (Hu et al., Nature 410:710-714 [2001]). Recently, IKKα was found to be required for B cell maturation, another unique function that is not provided by IKKβ (Kaisho et al., J. Exp. Med., 193:417-426 [2001]; and Senftleben et al., Science 293:1495-1499 [2001b]). This function of IKKα is dependent on its protein kinase activity, but instead of being mediated through inducible IκB degradation, it is exerted via a second NF-κB activation pathway, which is dependent on processing of the NF-κB2/p100 precursor protein to the mature p52 NF-κB subunit (Senftleben et al., [2001b], supra). This pathway requires the activity of another protein kinase, NIK (NF-κB inducing kinase), which may function as an activator of IKKα (Senftleben et al., supra [2001b]; and Yin et al., Science 291:2162-2167 [2001]).

One receptor-ligand pair postulated to mediate NF-κB activation during mammary gland development consists of RANK (receptor activator of NF-κB), a member of the TNF receptor (TNFR) family and RANK ligand (“RANKL,” also known as “OPGL,” “ODF,” and “TRANCE”), a member of the TNF family (Fata et al., Cell 103:41-50 [2000]). As its namesake indicates, RANK is an efficient NF-κB activator (Anderson et al., Nature 390:175-179 [1997]). Although RANK and RANKL were originally characterized as playing important roles in lymphocyte and osteoclast differentiation and activation (Kong et al., Nature 397:315-323 [1999]; and Lacey et al., Cell 93:165-176 [1998]), they were recently found to be essential for mammary gland development (Fata et al., supra). The biochemical pathway by which RANK controls mammary gland development has not been defined.

B. Generation of the Exemplary Ikkα^{AA/AA} Knockin Mice

Activation of IKKβ requires the phosphorylation of serines 177 and 181 in its activation loop (Delhase et al., supra; Mercurio et al., supra). The same serines are present in IKKα and in principle, were thought to have been required for its activation. Surprisingly, however, a S176A, S180A mutant of IKKα (Ikkα^{AA}) still supports TNFα or IL-1 mediated IKK activation in HeLa cells (Delhase et al., supra). These results indicated that IKKα may be involved. To identify such a function of IKKα in vivo, a mouse mutant expressing Ikkα^{AA} instead of the wt allele in all tissues was generated. In that mutant, a “knockin” Ikkα^{AA} allele was generated in which the two codons specifying the activation loop serines, whose phosphorylation is required for kinase activation (Delhase et al., Science 284:309-313 [1999]; and Mercurio et al., supra), were replaced with alanine codons.

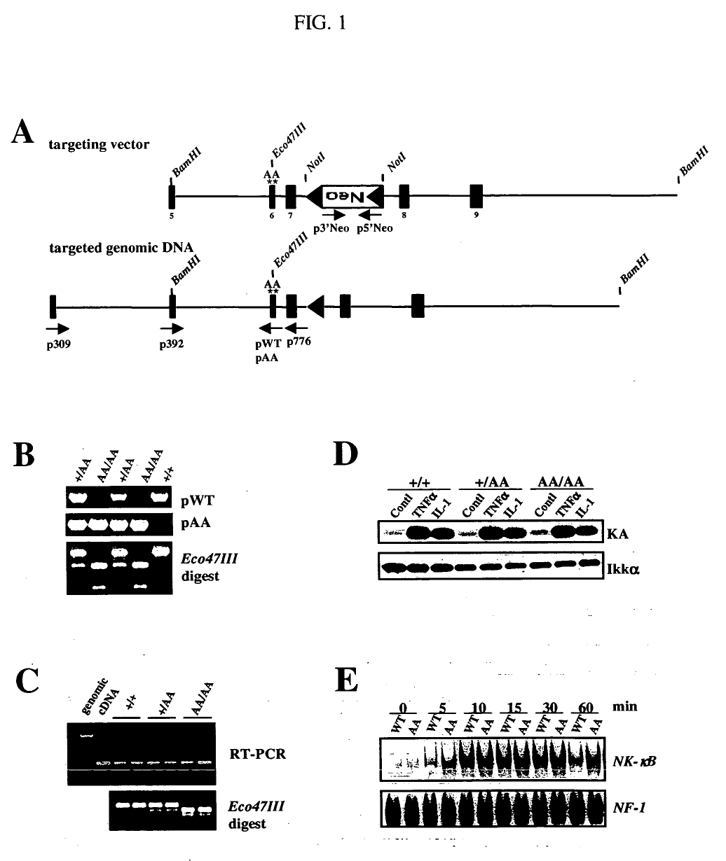

To replace serines 176 and 180 of IKKα with alanines, a NotI site was introduced into the 7^{th} intron of Ikkα, into which a loxP-flanked Neo^{r} cassette was inserted (See, FIG. 1, Panel A). Serine to alanine substitutions were introduced by site-directed mutagenesis, which also created an Eco47III site. A targeting vector containing these alterations was electroporated into embryonic stem (ES) cells and six clones with homologous recombination at the Ikkα locus were selected. To remove the Neo^{r} cassette, these ES clones were electroporated with a plasmid expressing Cre recombinase. A heterozygous Ikkα^{α/AA} ES clone was selected and injected into mouse blastocysts, yielding chimeric mice. These mice were bred to produce Ikkα^{+/AA} heterozygotes, which were intercrossed to generate homozygous Ikkα^{+/AA} knockin mice. The F₂ generation was genotyped by PCR analysis of tail DNAs using allele-specific primers and confirmed by Eco47III digestion (See, FIG. 1, Panel B). The frequency of Ikkα^{+/+}:Ikkα^{AA/AA}:Ikkα^{AA/AA} mice was approximately 1:2:1, as expected based on Mendelian inheritance. RT-PCR analysis of liver RNA demonstrated expression of the Ikkα^{AA}-allele (See, FIG. 1, Panel C). Sequencing of PCR products (using standard methods known in the art) confirmed the presence of the designed mutations. Ikkeα^{AA/AA} mice were normal from birth until adulthood and largely indistinguishable from their wt or heterozygote littermates in weight, size, appearance, development, fertility and behavior. In contrast to Ikk+^{-/-} mice (Hu et al., supra), Ikkα^{AA/AA} mice showed no defects in skin or limb development.

To further examine whether IKKα kinase activity contributes to IKK or NF-κB activation by proinflammatory cytokines, embryonic fibroblasts (MEF) were treated with TNFα or IL-1. Ikkα^{AA/AA} MEFs did not show any defects in IKK (See, FIG. 1, Panel D) or NF-κB (See, FIG. 1, Panel E) activation. IKK and NF-κB activation also proceeded normally in Ikkα^{AA/AA} mice administered endotoxin. These data are consistent with previous results, affirming that IKKβ is the major catalytic subunit responsible for IKK and NF-κB activation by proinflammatory stimuli.

C. IKKα is Essential for Lactation and Lobuloalveolar Development During Pregnancy as Demonstrated by the Transgenic Ikkα^{AA/AA} Animals of the Invention

In agreement with previous findings that IKKα kinase activity is not required for epidermal differentiation (Hu et al., [2001] supra), Ikkα^{AA/AA} mice are viable, healthy, fertile and morphologically normal. However, surprisingly, as described further herein and observed during the development of the present invention, the inventors discovered that Ikkα^{AA/AA} females display a severe lactation defect due to defective proliferation of the lobuloalveolar tree during pregnancy. Ikkα^{AA/AA} females completed pregnancy and gave birth to pups whose size and numbers were absolutely normal. However, all pups born to Ikkα^{AA/AA} mothers died within 1-2 days, although they exhibited normal nursing. Examination of the pups' stomachs revealed no milk. This phenotype was specific to Ikkα^{AA/AA} mothers and was independent of the pup genotype. When cross-fostering experiments were performed, no pups survived with Ikkα^{AA/AA} mothers, while pups nursed by Ikkα^{+/AA} or wt mothers did. These results confirmed that Ikkα^{AA/AA} mothers have a specific lactation defect that caused the lethality of their offspring. This defect was exhibited even after multiple pregnancies (at least 10).

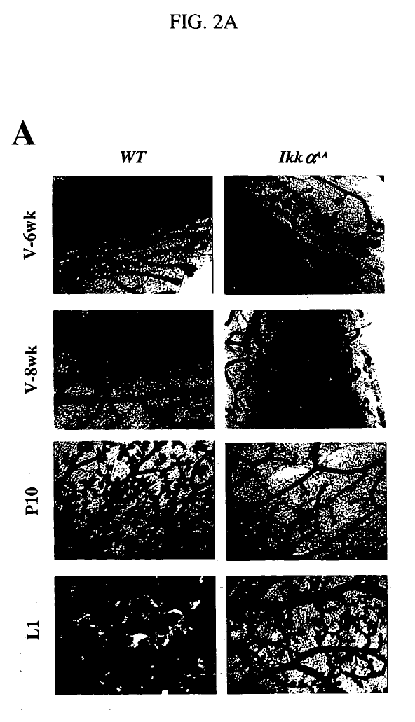

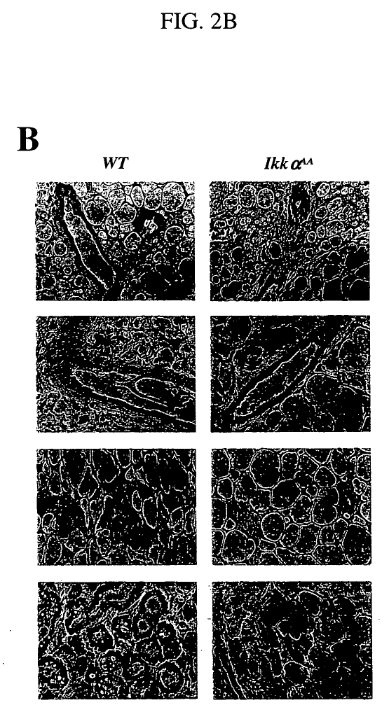

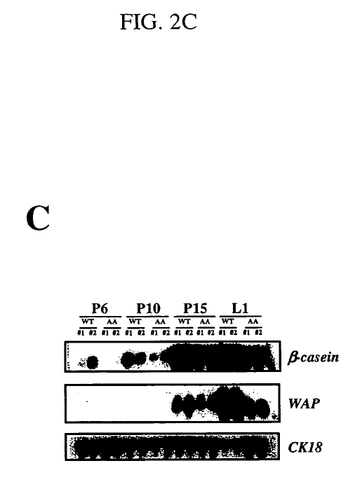

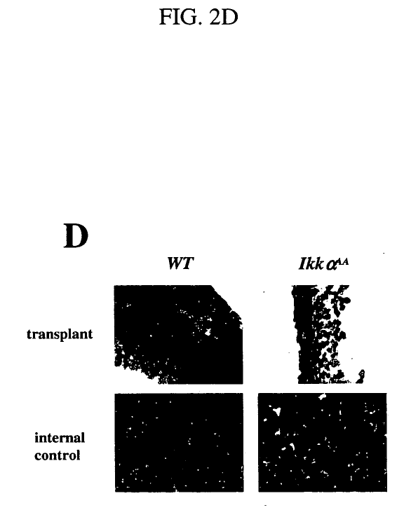

Wholemount analysis of mammary glands demonstrated that Ikkα^{AA/AA} virgin females completed normal ductal development (See, FIG. 2, Panel A). Surprisingly, however, examination on day 1 after delivery (L1), revealed that the lobuloalveolar tree in Ikkα^{AA/AA} mammary glands was severely undeveloped. Although some side branching and sprouting of mammary ducts were observed, their extent was comparable to that of wt mammary glands at 10 days of pregnancy (See, FIG. 2, Panel A). Histological analysis confirmed the failure of lobuloalveolar development in Ikkα^{AA/AA} mammary glands (See, FIG. 2, Panel B). The number of secretory alveoli was dramatically reduced in comparison to wt glands of same development age, and the alveoli of Ikkα^{AA/AA} glands were small with small or closed lumina. In summary, the extensive lobuloalveolar development which occurs during pregnancy, is severely impaired in Ikkα^{AA/AA} mice. Notably, mammary gland development in Ikkα^{AA/AA} females appeared to be normal during sexual maturation.

To evaluate the differentiation status of Ikkα^{AA/AA} mammary glands, expression of two milk protein genes, β-casein and whey acidic protein (WAP), was examined by Northern blotting. Milk protein genes were activated in Ikkα^{AA/AA} mammary glands, although at slightly lower levels relative to the wt (See, FIG. 2, Panel C), indicating that the differentiation process is not blocked.

Proper mammary gland development requires hormonal stimulation and epithelial-stromal interaction (Hennighausen and Robinson, supra; and Medina, supra). To determine whether the developmental defect of Ikkα^{AA/AA} mice was intrinsic to the mammary epithelium or due to either hormonal insufficiency or stromal cell defects, transplantation experiments were performed. Small pieces of mammary gland tissue taken from wt or Ikkα^{AA/AA} donors were implanted into cleared fat pads of 3 weeks old Ragl^{-/-} recipients. These mice were mated 9 weeks later and their mammary glands were analyzed shortly after delivery. Transplanted wt epithelia showed lobuloalveolar development similar to the internal control, while transplanted Ikkα^{AA/AA} epithelia failed to develop proper lobuloalveolar structures. Hence, the developmental defect of Ikkα^{AA/AA} mice was found to be autonomous to the mammary epithelium.

D. The Lactation Defect in The Exemplary Ikkα^{AA/AA} Mice is Due to Impaired Epithelial Cell Proliferation

To determine whether the mammary development defect was the result of reduced cell proliferation or increased cell death, BrdU incorporation and TUNEL assays were performed. Wt and Ikkα^{AA/AA} females at either 10 days of pregnancy (P10) or L1 were administered BrdU, whose incorporation into DNA was detected by immunohistochemistry. The proliferation index was calculated as the percentage of BrdU-positive alveolar cells per total epithelial cells for the samples (See, FIG. 3). At P10, the proliferation rate in the Ikkα^{AA/AA} mammary epithelium was approximately half of the wt rate (7.4% vs. 12.8%). At L1, the defect was even more dramatic (0.9% vs. 5.7%). It should be noted, however, that the number of epithelial cells was greatly reduced in the mutant glands. Similar results were obtained by staining sections of wt and Ikkα^{AA/AA} mammary glands with antibodies to proliferating cell nuclear antigen (PCNA), a marker for cells at the S phase of the cell cycle. The number of PCNA positive cells in the Ikkα^{AA/AA} mammary epithelium at P10 was 50% lower than in the wt. On the other hand, there were no significant differences in the apoptotic rates, measured by TUNEL assay, between wt and Ikkα^{AA/AA} mammary glands at either P10 or L1. Very few apoptotic cells could be detected at either time point. These results strongly indicate that IKKα is essential for proliferation of the lobuloalveolar epithelium in response to pregnancy signals.

E. Defective Cyclin D1 Induction in Ikkα^{AA/AA} Mammary Glands of the Invention's Transgenic Mice

Since cyclin D1 is the major G1 cyclin expressed in mammary epithelial cells, and cyclin D1^{-/-} females exhibit a similar defect in mammary gland development as Ikkα^{AA/AA} females (Fantl et al., supra; and Sicinski et al., supra), cyclin D1 expression in wt and Ikkα^{AA/AA} mammary glands was investigated. Immunohistochemical analyses revealed that at P10 only 20.0% of the Ikkα^{AA/AA} mammary epithelial cells were cyclin D1 positive, as compared to 42.4% of the wt cells (See, FIG. 4, Panel A). Similar results were revealed in immunoblot analyses which showed a reduction of approximately 50% in cyclin D1 levels at either P6 or P10 in Ikkα^{AA/AA} mammary glands (See, FIG. 4, Panel B). Similar results were obtained in RNA and microarray analyses, which also failed to reveal decreased expression of other cell cycle regulators. These results indicate that the proliferation defect in the Ikkα^{AA/AA} mammary epithelium is due to reduced cyclin D1 expression.

F. Defective NF-κB Activation in the Exemplary Ikkα^{AA/AA} Mammary Glands of the Invention's Transgenic Mice

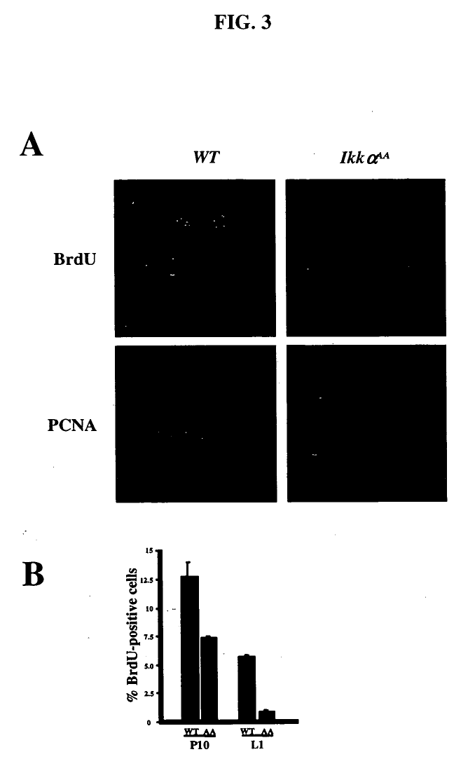

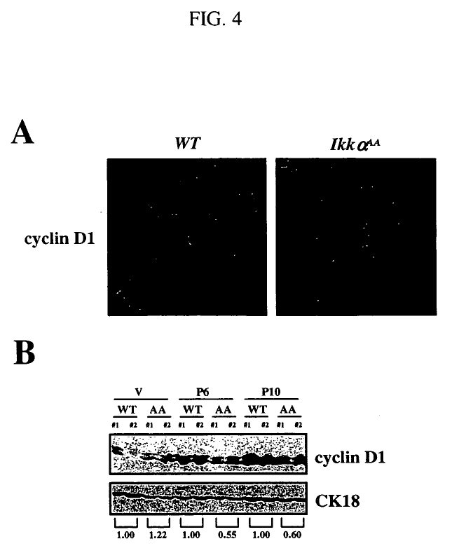

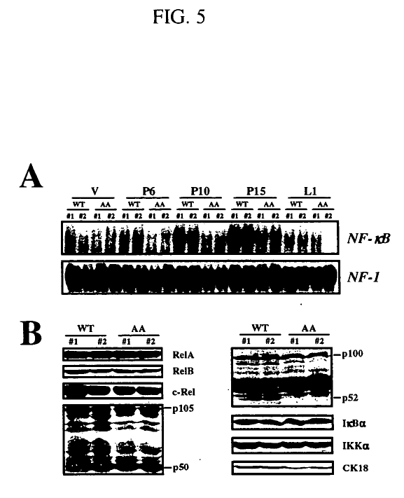

NF-κB DNA binding activity has been shown to be induced during pregnancy (Clarkson et al., supra; and Geymayer and Doppler, supra). In addition, cyclin DI has been suggested to be an NF-κB target gene (Guttridge et al., supra; and Hinz et al., supra). Thus, it was considered possible by the inventors that the reduced expression of cyclin D1 in Ikkα^{AA/AA} mammary glands may be caused by an NF-κB activation defect. Therefore, the induction of NF-κB DNA binding activity in wt and Ikkα^{AA/AA} mammary glands was monitored by gel mobility shift assay (See, FIG. 5, Panel A). Indeed, NF-κB activation in Ikkα^{AA/AA} mammary glands was almost completely diminished between P6 and L1. However, Western blot analyses revealed similar levels of NF-κB proteins, including RelA (p65), RelB, c-Rel, NF-κB1 p105, and p50, in wt and Ikkα^{AA} mammary glands analyzed at P10 (See, FIG. 5, Panel B). Consistent with previous results (Senftleben et al. [2001b], supra), processing of NF-κB2 p100 into mature p52 was defective in Ikkα^{AA/AA} mammary glands (See, FIG. 5, Panel B). It should be noted, however, that the total level of NF-κB2 in mammary glands is low, unlike the situation in B cells (Senftleben et al. [2001b], supra). Furthermore, no p52 could be detected even in wt NF-κB complexes, which were mostly p65:p50 heterodimers.

G. RANKL Activates NF-κB in Wild-Type, but not Ikkα^{AA/AA} Mammary Epithelial Cells of the Invention's Transgenic Mice

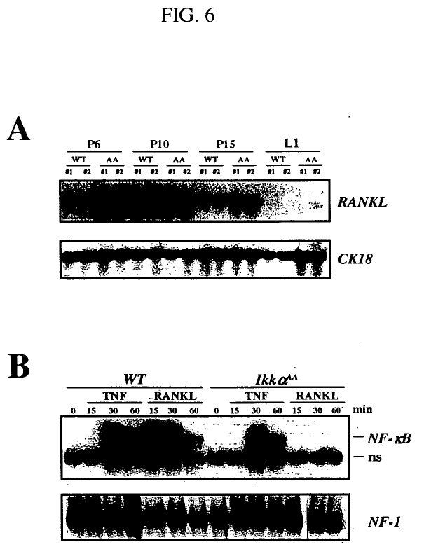

Defective mammary gland development, as exhibited by Ikkα^{AA/AA} and cyclin D1^{-/-} females, is exhibited by female mice lacking RANKL (OPGL/TRANCE/ODF) or its receptor RANK (Fata et al., supra). RANK ligation results in NF-κB activation (Anderson et al., supra). Therefore, it was considered possible by the inventors that the defect in mammary gland development caused by the Ikkα^{AA/AA} mutation is due to either diminished RANKL expression or defective RANK signaling. Northern blot analysis indicated that RANKL mRNA was actually elevated in Ikkα^{AA/AA} mammary glands between P6 and P15 (See, FIG. 6, Panel A). To examine RANK signaling in wt and Ikkα^{AA/AA} mammary epithelial cells, these cells were cultured using an established procedure (Pullan and Streuli, “The Mammary Epithelial Cell,” in Harris (ed.), “Epithelial Cell Culture,” Cambridge University Press, Cambridge, U.K. [1997], pages 97-121) and then stimulated with recombinant RANKL or TNFα, used as control. While TNFα induced NF-κB DNA binding activity in both wt and Ikkα^{AA/AA} epithelial cells, RANKL induced NF-κB activity (composed of p65:p50 heterodimers) in wt, but not Ikkα^{AA/AA} cells (See, FIG. 6, Panel B). These results were obtained at least three times with different primary cultures.

H. Inhibition of NF-κB Activity in Mammary Glands of Transgenic Mice Of The Invention Inhibits Their Development

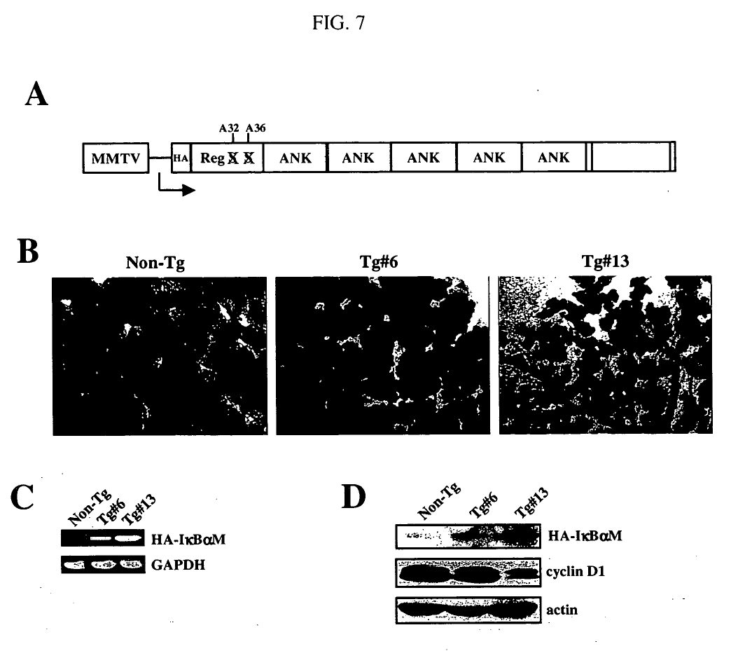

To assess whether the developmental defect in Ikkα^{AA/AA} mammary glands could be the direct consequence of diminished NF-κB activation, transgenic mice overexpressing the IκBα superrepressor mutant in their mammary epithelium were generated (See, FIG. 7, Panel A). This IκBα mutant contains alanines instead of serines 32 and 36 and therefore is no longer sensitive to IKK activation (DiDonato et al., Nature 388:548-554 [1997]). A similar construct was used to inhibit NF-κB activity in thymocytes (Hettmann et al., J. Exp. Med., 189:145-158 [1999]). Of two lines of MMTV-Iκbα^{A32/36} transgenic mice that were analyzed, females of line 13 exhibited a lactation defect similar to Ikkα^{AA/AA} mice, resulting in death of all newborns within 48 hours, while females of line 6 could lactate and nurse their pups.

Wholemount analysis of mammary glands taken at L1 confirmed a lobuloalveolar developmental defect in line 13 but not in line 6. Although both lines were positive for MMTV-Iκbα^{A}^{32/A36}, DNA expression of the transgene was considerably higher in line 13 (See, FIG. 7, Panel B). Correspondingly, expression of cyclin D1 was reduced in line 13 but not in line 6 mammary glands (See, FIG. 7, Panel C).

I. The Ikkα^{AA/AA} Mutant Phenotype of the Invention's Transgenic Mice is Rescued by a Cyclin D1 Transgene

The results presented above suggested to the inventors that the defect in lobuloalveolar development in Ikkα^{AA/AA} mammary glands is the result of a failure to properly induce cyclin D1 in response to activation of RANK or other receptors. Thus, assuming their hypothesis is correct, the inventors reasoned that restoration of cyclin D1 expression in Ikkα^{AA/AA} mammary glands should suppress the developmental defect and allow normal lactation to occur. Therefore, Ikkα^{AA/AA} males were crossed with MMTV-cyclin D1 transgenic females. The latter were shown to display mammary gland hyperplasia and increased susceptibility to development of breast tumors (Wang et al., Nature 369:669-671 [1994]). Ikka+MAMMTV-cyclin D1 heterozygotes were selected and intercrossed to generate Ikkα^{+/AA}MMMTV-cyclin D1 homozygous females which were mated and analyzed for lactation and mammary gland development. Expression of the MMTV-cyclin D1 transgene in the Ikk^{αAA/AA} genetic background was sufficient to completely suppress the lactation defect even after the first pregnancy. Wholemount analysis of mammary glands at L1 validated these results and revealed restoration of normal lobuloalveolar development by the MMTV-cyclin D1 transgene.

Immunoblot analysis indicated that expression of the MMTV-cyclin D1 transgene in the wt background resulted in only a modest elevation in cyclin D1, whereas its expression in the Ikkα^{AA/AA} background restored cyclin D1 expression to its normal level (See, FIG. 8, Panel A). However, the MMTV-cyclin D1 transgene did not restore NF-κB activity (See, FIG. 8, Panel B). These results strongly indicate that IKKα is a crucial intermediate in a signaling pathway through which RANK activation results in cyclin D1 induction, leading to increased proliferation of mammary epithelial cells (See, FIG. 8, Panel C). Defects in at least five components of this pathway result in the same outcome.

The results described above define a signaling pathway initiated by binding of the cytokine RANKL to its receptor RANK that culminates in induction of cyclin D1 in mammary gland epithelial cells. This pathway, composed of at least six components acting in a linear cascade: RANK, RANKL, IKKα, IkBα, NF-κB and cyclin D1 (See, FIG. 8, Panel C), is fully supported by genetic analysis and demonstrated to play a key role in controlling proliferation of the mammary epithelium during pregnancy. The results obtained during the development of the present invention establish the IKKα subunit of the IKK complex as a central intermediate in this pathway, responsible for cell type specific activation of NF-κB in response to RANK signaling. Remarkably, Ikkα^{AA/AA} mice are phenotypically normal, relatively healthy and fertile. The only perturbations found to be caused by this mutation, which destroys the activating phosphorylation sites of IKKα, are the defect in mammary gland development described herein and a partial defect in B cell maturation and formation of secondary lymphoid organs described elsewhere (Senftleben et al. [2001b], supra). Importantly, Ikkα^{AA/AA} mice do not display any of the morphogenetic defects caused by complete absence of IKKα (Hu et al. [1999]; and Takeda et al. [1999], supra) or the marked sensitivity to TNFα-induced apoptosis and deficient innate immune defenses caused by ablation of IKKβ (Li et al. Science 284:321-325 [1999]; Li et al., J. Exp. Med., 189:1839-1845 [1999]; Senftleben et al. [2001a], supra; and Tanaka et al. [1999], supra).

Based on the nature of the Ikkα^{AA} mutation, the function of IKKα in RANK signaling in mammary epithelial cells is likely to depend on its catalytic activity. However, an understanding of the mechanisms involved is not required in order to use the present invention. The Ikkα^{AA} mutation, however, does not interfere with NF-κB activation by TNFα in mammary epithelial cells or in response to TNFα, IL-1 or endotoxin in other cell types. The importance of these results is three-fold. First, they establish a well defined signaling pathway, fully supported by genetic analysis, that controls an organogenic process in mammals. Second, with the recent demonstration of cyclin D1 requirement for breast carcinogenesis (Yu et al., supra) and the highly specific and relatively mild pathology caused by the Ikkα^{AA} mutation, the present results provide compositions and methods for the development of specific therapies for breast cancer designed to be relatively free of toxic side effects. Third, the new results demonstrate that IKKα and IKKβ are suitable for differential deployment by different members of the TNF and TNFR families to induce IKB phosphorylation and NF-κB activation.

J. RANK, NF-κB and the Control of Mammary Gland Development

The genetic control of organogenesis has been extensively studied in organisms such as C. elegans and D. melanogaster. In many of the model systems studied, it has been established that one of the major signaling pathways controlling tissue growth and specification is the Ras-MAP kinase pathway activated by tyrosine kinase growth factor receptors (RTKs) (Sternberg and Han, Trends Genet., 14:466-472 [1998]; and Wassarman, Curr. Opin. Genet. Dev., 5:44-50 [1995]). Given the established importance of RTK signaling, it was somewhat of a surprise when a member of the TNF family, RANKL, and its receptor, RANK, were found to play a critical role in growth of the mammary epithelium and formation of its lobuloalveolar tree (Fata et al., supra). However, unlike the present invention, these initial findings, did not establish means by which RANK signaling controls mammary gland development.

The Ikkα^{AA} mutation causes the same defect in mammary gland development as the complete ablation of either RANK or RANKL (Fata et al., supra). In all three cases, the defect is restricted to growth of the lobuloalveolar tree during pregnancy. However, the Ikkα^{AA} mutation also results in defective NF-κB activation in mammary epithelial cells during pregnancy and in response to RANKL application. Furthermore, the specific inhibition of NF-κB in mammary epithelial cells via targeted expression of the IκBα superrepressor also prevents the expansion of the lobuloalveolar tree. It therefore follows that RANK signaling controls mammary gland development through an IKKα-dependent NF-κB activation pathway. Interestingly, however, the Ikkα^{AA} mutation does not result in osteopetrosis or any obvious effects on T cell development, as observed in Rank^{-/-} and Rankl^{-/-} mice (Kong et al., supra; and Lacey et al., supra).

The results obtained during the development of the present invention also establish the role of NT-κB in controlling proliferation of the mammary epithelium. Although experiments performed in transformed cell lines have suggested that cyclin D1 may be an NF-κB target gene (Guttridge et al., supra; and Hinz et al., supra) the experiments described herein are the first to establish an in vivo link between NF-κB and the cell cycle machinery. Previously, the loss of RANK signaling was suggested to cause a modest increase in apoptosis of mammary epithelial cells (Fata et al., supra). However, it was not possible to detect elevated apoptosis in Ikkα^{AA/AA} mammary glands during pregnancy. Although NF-κB is well established as an inhibitor of apoptosis (Beg and Baltimore, supra; Liu et al., supra; Van Antwerp et al., supra; and Wang et al., Science 274:787-789 [1996]), the apoptosis that ensues in its absence is not spontaneous and is dependent on TNFα signaling (Li et al. Science 284:321-325 [1999]; and Senftleben et al. [2001a], supra). Interestingly, increased growth of the mammary epithelium was also observed in transplanted mammary glands derived from IκBα-deficient mice (Brantley et al., Mol. Biol. Cell 12:1445-1455 [2001]), further lending support to the role of NF-κB in stimulating the proliferation of this tissue. The inhibition of NF-κB activation, either by the Ikkα^{AA} mutation or via cell-type specific expression of the IκBα superrepressor, results in decreased expression of cyclin D1. Most importantly, restoration of cyclin D1 to its normal level was found to completely suppress the defect in mammary gland development in Ikkα^{AA/AA} mice, thereby providing strong genetic evidence of the critical role of the IKKα-dependent cyclin D1 induction pathway.

Although most members of the TNF family are regulators of innate and adaptive immune responses (Locksley et al., Cell 104:487-501 [2001]), the results obtained during the development of the present invention firmly establish the importance of RANKL, a member of this family, in the formation of an epithelial-based tissue. Recently, members of the TNFR family, EDAR and XEDAR and their ligand EDA, were shown to be involved in hair follicle development (Headon and Overbeek, Nat. Genet., 22:370-374 [1999]), but the exact cellular and biochemical mechanisms through which they act remain obscure. Nonetheless, it is becoming increasingly clear that members of thc TNF and TNFR families regulate many biological processes.

Whereas the results described herein illustrate the existence of a linear signaling cascade through which RANKL activates IKKα and NF-κB to induce cyclin D1 in mammary epithelial cells (See, FIG. 8, Panel C), this pathway is likely to be subjected to more intricate regulation. For instance, it is contemplated that the synthesis of RANKL in mammary epithelial cells is regulated by hormonal cues and cell-cell interactions (Fata et al., supra). It is further contemplated that RANKL expression is also subjected to negative feedback from NF-κB, as suggested by its elevated expression in Ikkα^{AA/AA} mammary glands and transient expression in wt glands. NF-κB activity itself is also under negative regulation and the cyclin D1 promoter contains multiple cis elements, including an AP-1 site, that allow the integration of additional signaling cascades, such as those activated by RTKs. However, an understanding of the mechanisms involved is not necessary in order to use the present invention.

K. The Many Functions of IKKα

Structurally and biochemically, IKKα is very similar to IKKβ, except for its lower IκB kinase activity (Rothwarf and Karin, supra). Therefore, it was a surprise when IKKα-deficient cells and mice were found to have rather minor defects in NF-κB activation in response to proinflammatory stimuli in the face of the major defect caused by the absence of IKKβ (Rothwarf and Karin, supra). Although IKKα is uniquely required for keratinocyte differentiation and formation of the epidermis (Hu et al. [1999], supra; and Takeda et al., supra), this function does not depend on its protein kinase activity or NF-κB (Hu et al. [2001], supra).

Recently, IKKα was found to have a second function that does depend on its protein kinase activity—the processing of NF-κB2 p100 to the mature p52 DNA binding subunit (Senftleben et al. [2001b], supra). Although IKKα kinase activity is also required for p100 processing in mammary epithelial cells, it is unlikely that a p52 deficiency accounts for the defect in mammary gland development. First, unlike B cells where it is expressed in relatively high levels (Senftleben et al. [2001b], supra), p52 is only a minor component of NF-κB in mammary epithelial cells. Second, lactation defects have not been reported for NF-κB2 deficient mice (Caamano et al., J. Exp. Med., 187:185-196 [1998]; and Franzoso et al., J. Exp. Med., 187:147-159 [1998]).

It is contemplated by the inventors that the proliferation of mammary epithelial cells and cyclin D1 induction during pregnancy are dependent on the classical NF-κB activation pathway. Indeed, the loss of IκBα results in increased growth of the mammary epithelium (Brantley et al., Mol. Biol. Cell 12:1445-1455 [2001]) and a general reduction in NF-κB activity phenocopies the Ikkα^{AA} mutation. Thus, IKKα has a third essential function—the phosphorylation of IκBα in response to specific signals.

Although IKKα was expected by the inventors to be an IκB kinase, the finding that its kinase activity is required for NF-κB activation only in response to distinct stimuli is novel and highly surprising. However, an understanding of the mechanisms involved is not necessary in order to use the present invention.

Based on biochemical analysis of two human cell lines (HeLa and Jurkat), the IKK complex was found to be composed of IKKα:IKKβ heterodimers, that are assembled into a heterotetramer through IKKγ-mediated dimerization (Rothwarf and Karin, supra). Given the tight interaction between IKKα and IKKβ and their strong autophosphorylation activity, it is counterintuitive that the two catalytic subunits can be differentially activated. Surprisingly, the present results strongly suggest that in mammary epithelial cells, IKKα and IKKβ are differentially regulated. Whereas Ikkα^{AA/AA} cells show normal NF-κB activation in response to TNFα, presumably through IKKβ, they are non-responsive to RANKL. Thus, IKKα rather than IKKβ is required for NF-κB activation by this TNF family member.

While an understanding of the mechanism is not necessary to practice the instant invention, the simplest mechanism that could account for differential IKKα and IKKβ activation entails the existence of two separate IKK complexes, one corresponding to the classical IKKα:IKKβ:IKKγ complex, and the other an IKKα:IKKα:IKKγ complex. This situation could arise through overproduction of the IKKα subunit in a particular cell type. However, the relative expression of IKKα in mammary epithelial cells has not been found to be different from that in mouse fibroblasts. Another possible mechanism entails the existence of two IKK-kinases, one involved in IKKβ activation by TNFα, IL-1 or endotoxin, and the other responsible for IKKα activation by RANKL. It was previously thought that NIK may represent a general IKK-kinase responsible for activation by most stimuli (Woronicz et al., Science 278:866-869 [1997]). However, the recent knockout of the Nik gene demonstrated that it is not required for NF-κB activation by most stimuli, with the exception of lymphotoxin β (LTβ), a member of the TNF family (Yin et al., supra). During the development of the present invention, evidence was found that NIK may be a specific IKKα activator (Senftleben et al. [2001b], supra). Although Nik^{-/-} mice were reported to exhibit normal lactation (Yin et al., supra), it should be noted that aly mice, which carry a point mutation in the Nik gene that interferes with kinase activation (Shinkura et al., Nat. Genet., 22:74-77 [1999]), were found to exhibit defective mammary gland development, similar to Ikkα^{AA/AA} mice (Nishimura et al., Am. J. Reprod. Immunol., 43:351-358 [2000]). Thus, it is contemplated that NIK may be a specific IKKαx-kinase involved in its activation by a subset of the TNF and TNFR families. However, an understanding of the mechanism(s) involved is not necessary in order to use the present invention.

Although the details of IKK activation by any one stimulus, including TNFα, remain to be elucidated, the present results demonstrate for the first time that IKKα and IKKβ differ in their responsiveness to upstream stimuli. These differences find use in the design of therapeutically useful IKK inhibitors. Furthermore, an understanding of the mechanism(s) involved is not necessary in order to use the present invention. While selective IKKβ inhibition is expected to inhibit inflammatory responses, it is further contemplated to block innate immune responses and greatly enhance the sensitivity of various cells to TNFα-induced apoptosis ((Li et al. Science 284:321-325 [1999]; Li et al., J. Exp. Med., 189:1839-1845 [1999]; and Senftleben et al. [2001a], supra). In contrast to selective IKKβ inhibitors, a selective IKKα inhibitor, on the other hand, is contemplated to inhibit B cell maturation and block the proliferation of mammary epithelial cells, but leave innate immunity as well as T-cell mediated adaptive immunity intact. Such an inhibitor should not sensitize cells to TNFα-induced apoptosis. Given the common occurrence of constitutive NF-κB in breast tumors (Cogswell et al., supra; Nakshatri et al., Mol. Cell Biol., 17:3629-3639 [1997]; and Sovak et al., J. Clin. Invest., 100:2952-2960 [1997]) and their reliance on cyclin D1 (Yu et al., Nature 411:1017-1021 [2001]), it is contemplated that at least some breast cancer cases will be treatable with selective IKKα inhibitors without causing the severe side effects associated with inhibition of IKKβ.

L. Reducing Mammary Epithelial Cell Proliferation

The invention provides methods for reducing mammary epithelial cell proliferation in a tissue, comprising administering an agent that specifically reduces IKKα kinase activity to a tissue that contains mammary epithelial cells such that mammary epithelial cell proliferation in the treated tissue is reduced.

The term “cell proliferation” as used herein refers to the physiological and morphological progression of changes that cells undergo when dividing. The cell cycle is generally recognized to be composed of phases termed “interphase,” “prophase,” “metaphase,” “anaphase,” and “telophase.” Additionally, parts of the cell cycle may be termed “M (mitosis),” “S (synthesis),” “G0,” “G1 (gap 1)” and “G2 (gap2).” Furthermore, the cell cycle includes periods of progression that are intermediate to the above named phases.

The terms “reducing mammary epithelial cell proliferation,” “inhibiting mammary epithelial cell proliferation,” “diminishing mammary epithelial cell proliferation,” “suppressing mammary epithelial cell proliferation,” and grammatical equivalents thereof, refer to reducing the level of mammary epithelial cell proliferation by any amount that is statistically significant using any art-accepted statistical method of analysis in a preferred embodiment, reduced mammary epithelial cell proliferation in a sample refers to a quantity of mammary epithelial cell proliferation that is preferably at least 10% less than, more preferably at least 50% less than, yet more preferably at least 75% less than, even more preferably at least 90% less than, the quantity in a control sample, and most preferably is at the same level which is observed in a control sample. A reduced level of mammary epithelial cell proliferation need not, although it may, mean an absolute absence of mammary epithelial cell proliferation. The invention does not require, and is not limited to, methods that wholly eliminate mammary epithelial cell proliferation.

The level of mammary epithelial cell proliferation may be determined using methods well known in the art. For example, cells may be incubated with bromodeoxyuride (BrdU), which is incorporated into the DNA of dividing cells, followed by detecting BrdU incorporation into DNA by immunohistochemistry. As disclosed herein, the proliferation index may calculated as the percentage of BrdU-positive alveolar cells per total epithelial cells for the samples (See, FIG. 3). Alternatively, the level of mammary epithelial cell proliferation may be determined by staining tissue sections with antibodies to proliferating cell nuclear antigen (PCNA), which is a marker for cells at the S phase of the cell cycle, followed by counting the number of PCNA positive cells in the tissue.

The invention's methods are useful where the sample is in vitro (e.g., a biopsy from a subject, or cultured cells) as well as in a tissue in vivo (i.e., that is in a subject).

i. Agents that Specifically Reduce IKKα Kinase Activity

The methods of the present invention include the step of utilizing an agent that specifically reduces IKKα kinase activity.

The terms “IKKα kinase activity” and “IKKα protein kinase activity” are used interchangeably herein to refer to the biochemical activity of phosphorylation of at least one of the substrates GST-IκBα (amino acids 1 to 54), full-length GST-IκBα, GST-IκBβ (amino acids 1-44) or full-length GST-IκBα. Methods for assaying IKKα kinase activity are known in the art (DiDonato et al., Nature 38:548-554 [1997]) and disclosed herein using commercially available IKKγ antibody (PharMingen).

The terms “specific” and “specifically” as used herein in reference to an agent's effect on IKKα kinase activity, or an agent's interaction with IKKα, means that the agent's effect or interaction is dependent upon the presence of a particular structure on IKKα. For example, if an agent is specific for epitope “A,” the presence of a protein containing epitope A (or free, unlabelled A) in a reaction containing labeled “A” and the agent will reduce the amount of labeled A bound to the agent.

The terms “reduce IKKα kinase activity,” “diminish IKKα kinase activity,” and “inhibit IKKα kinase activity” are used interchangeably when in reference to the effect of an agent on IKKα kinase activity, and mean that the agent reduces the level of IKKα kinase activity in a first sample relative to a second sample by any amount that is statistically significant using any art-accepted statistical method of analysis. In a preferred embodiment, reduced IKKα kinase activity in a sample refers to a quantity of IKKα kinase activity that is preferably at least 10% less than, more preferably at least 50% less than, yet more preferably at least 75% than, even more preferably at least 90% less than, the quantity in a control sample, and most preferably is at the same level which is observed in a control sample. A reduced level of IKKα kinase activity need not, although it may, mean an absolute absence of IKKα kinase activity. The invention does not require, and is not limited to, methods that wholly eliminate IKKα kinase activity. For example, reduced IKKα kinase activity in a first sample relative to a second sample can mean that the first sample contains only basal IKKα kinase activity, whereas the second sample contains basal IKKα kinase activity as well as activated IKKα kinase activity.

The term “specifically reduces” when in reference to the effect on a specified enzyme activity (such as IKKα kinase activity) means that the reduced activity is attained “in the absence of” an (i.e., without a statistically significant) effect on another phenomenon. For example, an agent that “specifically reduces IKKα kinase activity” means that the agent reduces IKKα kinase activity in the absence of an effect on the development of skin and/or limbs, as determined by, for example, histological or clinical tests.

The terms “agent,” “test agent,” “molecule,” “test molecule,” “compound,” and “test compound” as used herein refer to any type of molecule (for example, a peptide, nucleic acid, carbohydrate, lipid, organic, and inorganic molecule, etc.) obtained from any source (for example, plant, animal, and environmental source, etc.), or prepared by any method (for example, purification of naturally occurring molecules, chemical synthesis, and genetic engineering methods, etc.). Agents are exemplified by, but not limited to, antibodies, nucleic acid sequences, and other agents as further described below.

1. Antibodies

In one preferred embodiment, the agent is an antibody that specifically binds to an IKKα domain. The terms “antibody” and “immunoglobulin” are interchangeably used to refer to a glycoprotein or a portion thereof (including single chain antibodies), which is evoked in an animal by an immunogen and which demonstrates specificity to the immunogen, or, more specifically, to one or more epitopes contained in the immunogen. The term “antibody” includes polyclonal antibodies, monoclonal antibodies, naturally occurring antibodies as well as non-naturally occurring antibodies, including, for example, single chain antibodies, chimeric, bifunctional and humanized antibodies, as well as antigen-binding fragments thereof, including, for example, Fab, F(ab′)₂, Fab fragments, Fd fragments, and Ev fragments of an antibody, as well as a Fab expression library. It is intended that the term “antibody” encompass any immunoglobulin (e.g., IgG, IgM, IgA, IgE, IgD, etc.) obtained from any source (e.g., humans, rodents, non-human primates, caprines, bovines, equines, ovines, etc.). The term “polyclonal antibody” refers to an immunoglobulin produced from more than a single clone of plasma cells; in contrast “monoclonal antibody” refers to an immunoglobulin produced from a single clone of plasma cells. Monoclonal and polyclonal antibodies may or may not be purified. For example, polyclonal antibodies contained in crude antiserum may be used in this unpurified state.

Antibodies useful in the instant invention include, without limitation, “anti-IKKα antibodies,” i.e., antibodies that specifically bind to IKKα polypeptide, and “anti-IKKα kinase domain antibodies,” i.e., antibodies that specifically bind to the IKKα kinase domain. Exemplary antibodies include, without restriction, anti-human IKKα kinase domain antibodies; humanized anti-human IKKα kinase domain antibodies; chimeric antibodies that specifically bind to the IKKα kinase domain, and that are generated using methods disclosed by Newman et al., U.S. Pat. No. 5,750,105, the contents of which are incorporated by reference; rat anti-mouse IKKα kinase domain antibodies that may be generated using methods known in the art [e.g., Chisholm et al. (1993) European J. Immunol 23: 682-688]; mouse anti-rat IKKα kinase domain antibodies that may be generated using methods known in the art [e.g., Issekutz (1991) J. Immunol 147:4178-4184]; and rat anti-mouse IKKα kinase domain antibodies that may be generated using art known methods [e.g., Holzmann et al. (1989) Cell 56: 37-46].

Commercially available antibodies that may be useful in the invention's methods include anti-IKKα antibodies such as, without limitation, monoclonal antibodies from BD PharMingen, San Diego Calif., cat# 556532; several antibodies from Santa Cruz Biotechnology, Inc. Santa Cruz CA, for example, cat# sc-7606 (monoclonal), cat# sc-7121 (polyclonal against C-terminus), cat# sc-7218 (polyclonal), cat# sc-7183 (polyclonal against mouse amino acids 465-745), cat# sc-7190 (polyclonal), and cat# sc-7184 (polyclonal against mouse amino acids 248-452); and antibodies from Imgenex, San Diego Calif., such as cat# IMG-136 (monoclonal).

In a preferred embodiment, the antibody is an “anti-phosphoserine antibody” which refers to an antibody that is specific for phosphorylated serine. These antibodies are useful in detecting activated IKKα kinase, since the activated IKKα kinase contains phosphoserine, whereas in activated IKKα does not. Anti-phosphoserine antibodies are commercially available such as from New England Bioloab, catalog #2681, and can be made using standard methods as described herein. One of skill in the art recognizes that since an anti-phosphoserine antibody may bind to both activated IKKα and activated IKKα, the presence and/or quantity of binding of the anti-phosphoserine antibody to IKKα may be distinguished from its binding to IKKβ by relying on the difference in molecular weight of IKKα and IKKβ. For example, the antibody that is bound to IKKα may be resolved from the antibody that is bound to IKKβ by SDS-polyacrylamide gel electrophoresis, followed by detection of the bound antibody (e.g., by detecting fluorescence, radiolabel, etc.).

Naturally occurring antibodies may be generated in any species including murine, rat, rabbit, hamster, human, and simian species using methods known in the art. Non-naturally occurring antibodies can be constructed using solid phase peptide synthesis, can be produced recombinantly or can be obtained, for example, by screening combinatorial libraries consisting of variable heavy chains and variable light chains as previously described [Huse et al., Science 246:1275-1281 (1989)]. These and other methods of making, for example, chimeric, humanized, CDR-grafted, single chain, and bifunctional antibodies are well known to those skilled in the art (Winter and Harris, Immunol. Today 14:243-246 (1993); Ward et al., Nature 341:544-546 (1989); Hilyard et al., Protein Engineering: A practical approach (IRL Press 1992); and Borrabeck, Antibody Engineering, 2d ed. (Oxford University Press 1995).

The term “specifically binds” and “specific binding” are in reference to the binding of an antibody to an IKKα kinase domain refer to an interaction of the antibody with one or more epitopes on an IKKα kinase domain, where the interaction is dependent upon the presence of a particular structure on the IKKα kinase domain. For example, if an antibody is specific for epitope “A” on an IKKα kinase domain, then the presence of a protein containing epitope A (or free, unlabelled A) in a reaction containing labelled “A” and the antibody will reduce the amount of labelled A bound to the antibody.

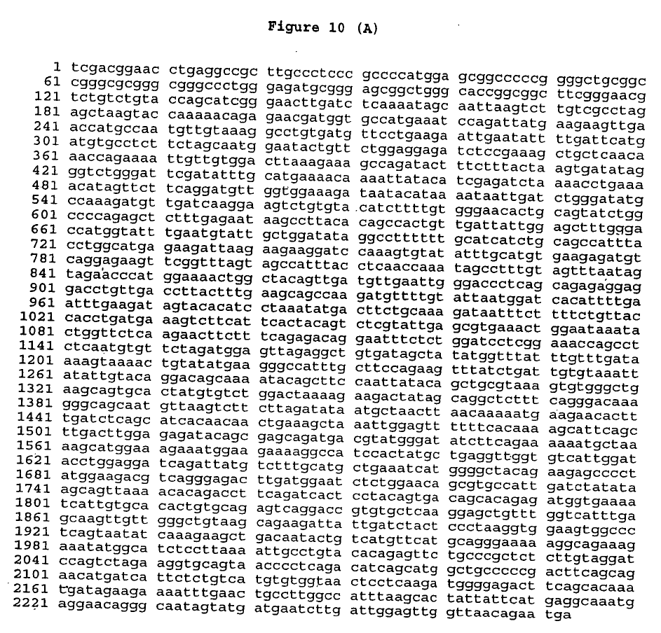

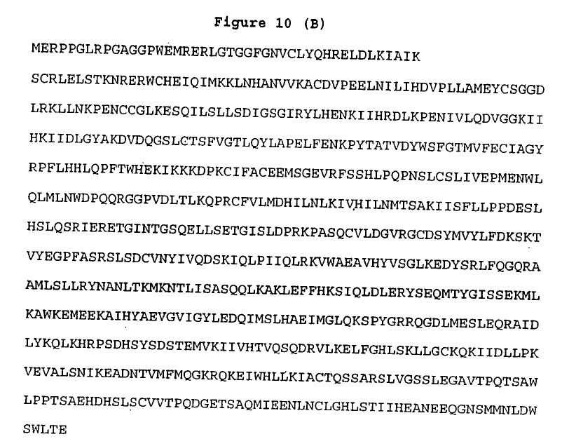

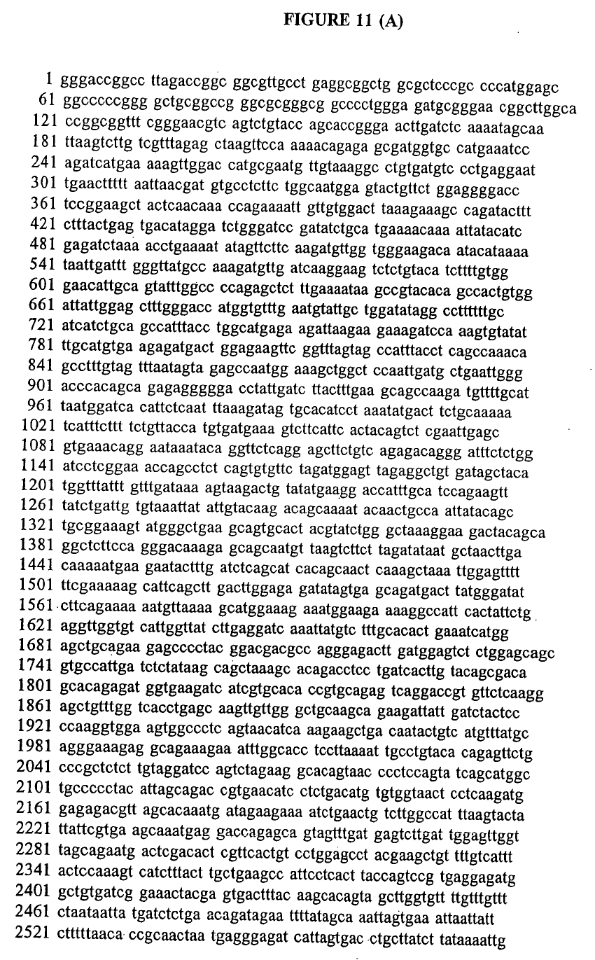

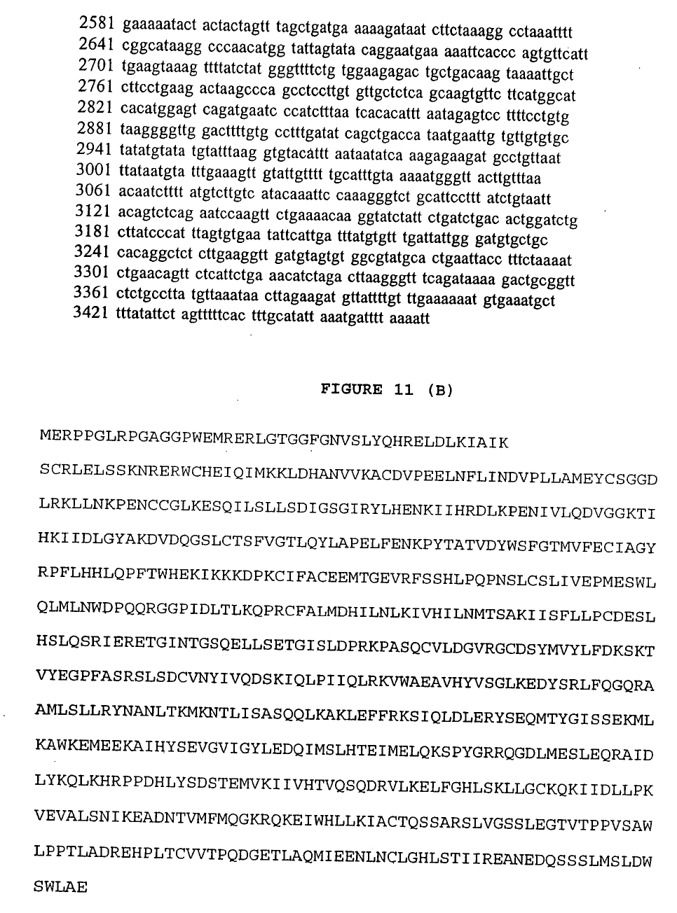

The terms “IKKα kinase domain,” “IKKα protein kinase domain,” and “IKKα protein kinase catalytic domain” are used interchangeably herein to refer to amino acids 1 to 301 of the human IKKα kinase (SEQ ID NO:13) (FIG. 10), and/or to amino acids 1 to 301 of the mouse IKKα kinase (SEQ ID NO:15) (FIG. 11), and/or portions thereof that exhibit IKKα kinase activity.

In one embodiment, the invention's antibodies are characterized by having specific binding activity for the IKKα kinase domain of at least about 1×10⁵M⁻¹, more preferably at least about 1×10⁶M⁻¹, and yet more preferably at least about 1×10⁷M⁻¹.

Those skilled in the art know how to make polyclonal and monoclonal antibodies which are specific to a desirable polypeptide. For the production of monclonal and polyclonal antibodies, various host animals can be immunized by injection with the peptide corresponding to any molecule of interest in the present invention, including but not limited to rabbits, mice, rats, sheep, goats, etc. In one preferred embodiment, the peptide is conjugated to an immunogenic carrier (e.g., diphtheria toxoid, bovine serum albumin (BSA), or keyhole limpet hemocyanin [KLH]). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies directed toward molecules of interest in the present invention, any technique that provides for the production of antibody molecules by continuous cell lines in culture may be used (See, e.g., Harlow and Lane, Antibodies: A Laboratory Manual, Cold Spring Harbor Laboratory Press, Cold Spring Harbor, N.Y.). These include but are not limited to the hybridoma technique originally developed by Kohler and Milstein (Köhler and Milstein, Nature 256:495-497 [1975]), as well as the trioma technique, the human B-cell hybridoma technique (See e.g., Kozbor et al. Immunol. Today 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]). In some particularly preferred embodiments of the present invention, the present invention provides monoclonal antibodies of the IgG class.

In additional embodiments of the invention, monoclonal antibodies can be produced in germ-free animals utilizing technology such as that described in PCT/US90/02545. In addition, human antibodies may be used and can be obtained by using human hybridomas (Cote et al., Proc. Natl. Acad. Sci. U.S.A.80:2026-2030 [1983]) or by transforming human B cells with EBV virus in vitro (Cole et al., in Monoclonal Antibodies and Cancer Therapy, Alan R. Liss, pp. 77-96 [1985]).

Furthermore, techniques described for the production of single chain antibodies (See e.g., U.S. Pat. No. 4,946,778; herein incorporated by reference) can be adapted to produce single chain antibodies that specifically recognize a molecule of interest (e.g., at least a portion of IKK, as described herein). An additional embodiment of the invention utilizes the techniques described for the construction of Fab expression libraries (Huse et al., Science 246:1275-1281 [1989]) to allow rapid and easy identification of monoclonal Fab fragments with the desired specificity for a particular protein or epitope of interest (e.g., at least a portion of IKK).

The invention also contemplates humanized antibodies. Humanized antibodies may be generated using methods known in the art, including those described in U.S. Pat. Nos. 5,545,806; 5,569,825 and 5,625,126, the entire contents of which are incorporated by reference. Such methods include, for example, generation of transgenic non-human animals which contain human immunoglobulin chain genes and which are capable of expressing these genes to produce a repertoire of antibodies of various isotypes encoded by the human immunoglobulin genes.

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al., Science, 246:1275-1281 [1989]) to allow rapid and easy identification of monoclonal Fab fragments with the desired specificity.

Antibody fragments that contain the idiotyLpe (antigen binding region) of the antibody molecule can be generated by known techniques. For example, such fragments include but are not limited to: the F(ab′)₂ fragment that can be produced by pepsin digestion of an antibody molecule; the Fab′ fragments that can be generated by reducing the disulfide bridges of an F(ab′)₂ fragment, and the Fab fragments that can be generated by treating an antibody molecule with papain and a reducing agent.

In the production of antibodies, screening for the desired antibody can be accomplished by techniques known in the art (e.g., radioimmunoassay, ELISA [enzyme-linked immunosorbent assay], “sandwich” immunoassays, immunoradiometric assays, gel diffusion precipitin reactions, immunodiffusion assays, in situ immunoassays [e.g., using colloidal gold, enzyme or radioisotope labels], Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. As is well known in the art, the immunogenic peptide should be provided free of the carrier molecule used in any immunization protocol. For example, if the peptide was conjugated to KLH, it may be conjugated to BSA, or used directly, in a screening assay.

The foregoing antibodies can be used in methods known in the art relating to the localization and structure of compounds such as IKK and/or IKK subunits (e.g., for Western blotting to detect IKKβ, IKKα, etc.), and measuring levels thereof in appropriate biological samples, etc. For example, the antibodies can be used to detect proteins of interest in a biological sample from an individual.

The biological samples can then be tested directly for the presence of a protein of interest (e.g., IKK and its subunits) using an appropriate immunoassay strategy. Alternatively, proteins in the sample can be size separated (e.g., by polyacrylamide gel electrophoresis (PAGE), in the presence (or absence) of sodium dodecyl sulfate (SDS), and the presence of the protein of interest is then detected by immunoblotting (Western blotting).

The foregoing explanations of particular assay systems are presented herein for purposes of illustration only, in fulfillment of the duty to present an enabling disclosure of the invention. It is to be understood that a variety of immunochemical assay protocols are encompassed within the spirit and scope of the present invention.

2. Nucleic Acid Sequences

In an alternative embodiment, the agent that specifically reduces IKKα kinase activity is a nucleic acid sequence. The terms “nucleic acid sequence” and “nucleotide sequence” as used herein refer to two or more nucleotides which are covalently linked to each other. Included within this definition are oligonucleotides, polynucleotide, and fragments or portions thereof, DNA or RNA of genomic or synthetic origin which may be single- or double-stranded, and represent the sense or antisense strand. Nucleic acid sequences which are particularly useful in the instant invention include, without limitation, antisense sequences and ribozymes.

a. Antisense

Antisense sequences have been successfully used to inhibit the expression of several genes [Markus-Sekura (1988) Anal. Biochem. 172:289-295; Hambor et al. (1988) J. Exp. Med. 168:1237-1245; and patent EP 140 308], including the gene encoding VCAMI, one of the integrin α4β1 ligands [U.S. Pat. No. 6,252,043, incorporated in its entirety by reference]. The terms “antisense DNA sequence” and “antisense sequence” as used herein interchangeably refer to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex which is transcribed by a cell in its natural state into a “sense mRNA.” Sense mRNA generally is ultimately translated into a polypeptide. Thus, an “antisense DNA sequence” is a sequence which has the same sequence as the non-coding strand in a DNA duplex, and which encodes an “antisense RNA” (i.e., a ribonucleotide sequence whose sequence is complementary to a “sense mRNA” sequence). The designation (−) (i.e., “negative”) is sometimes used in reference to the antisense strand, with the designation (+) sometimes used in reference to the sense (i.e., “positive”) strand. Antisense RNA may be produced by any method, including synthesis by splicing an antisense DNA sequence to a promoter which permits the synthesis of antisense RNA. The transcribed antisense RNA strand combines with natural mRNA produced by the cell to form duplexes. These duplexes then block either the further transcription of the mRNA or its translation, or promote its degradation.

Any antisense sequence is contemplated to be within the scope of this invention if it is capable of reducing the level of expression of IKKα, and more particularly the level of expression of the IKKα kinase domain to a quantity which is less than the quantity of IKKα kinase domain expression in a corresponding control tissue which is (a) not treated with the antisense IKKα kinase domain sequence, (b) treated with a corresponding sense IKKα kinase domain sequence, or (c) treated with a nonsense sequence.

The terms “reducing the level of expression of IKKα kinase domain,” “diminishing IKKα kinase domain expression” and grammatical equivalents thereof, refer to reducing the level of IKKα kinase domain expression to a quantity which is preferably at least 20% less than the quantity in a corresponding control tissue, more preferably is at least 50% less than the quantity in a corresponding control tissue, yet more preferably is at least 90% less than the quantity in a corresponding control tissue, and most preferably is at the background level of, or is undetectable by, a Western blot analysis of IKKα kinase domain, by immunofluorescence for detection of IKKα kinase domain, by reverse transcription polymerase chain (RT-PCR) reaction for detection of IKKα kinase domain mRNA, or by in situ hybridization for detection of IKKα kinase domain mRNA. When a background level or undetectable level of IKKα kinase domain peptide or mRNA is measured, this may indicate that IKKα kinase domain is not expressed. A reduced level of IKKα kinase domain need not, although it may, mean an absolute absence of expression of IKKαt kinase domain. The invention does not require, and is not limited to, antisense IKKα kinase domain sequences which eliminate expression of IKKα kinase domain.

In one preferred embodiment, the antisense sequence is an IKKα kinase domain antisense sequence. The term “IKKα kinase domain antisense sequence” refers to an antisense sequence that is capable of hybridizing with at least a portion of IKKα kinase domain cDNA under high stringency or low stringency conditions, more preferably under high stringency conditions. The term “IKKα kinase domain antisense sequence” also can refer to an antisense sequence that has at least 51%, preferably at least 70%, more preferably at least 80%, yet more preferably at least 90%, and most preferably 100%, homology to the sense strand of dsDNA encoding IKKα kinase domain.

In another embodiment, the antisense sequence is an IKKα antisense sequence. The term “IKKα antisense sequence” refers to an antisense sequence that is capable of hybridizing with at least a portion of IKKα cDNA under high stringency or low stringency conditions, more preferably under high stringency conditions. The term “IKKα antisense sequence” also can refer to an antisense sequence that has at least 51%, preferably at least 70%, more preferably at least 80%, yet more preferably at least 90%, and most preferably 100%, homology to the sense strand of dsDNA encoding IKKα.

Antisense sequences within the scope of this invention may be designed using approaches known in the art. In a preferred embodiment, the antisense sequences are designed to be hybridizable to at least a portion of an mRNA that is encoded by the coding region of the IKKα gene, and in particular, to at least a portion of an mRNA encoding at least a portion of an IKKα kinase domain. Alternatively, while less preferred, antisense sequences may be designed to reduce transcription by hybridizing to upstream nontranslated sequences, thereby preventing promoter binding to transcription factors.

In a preferred embodiment, the antisense oligonucleotide sequences of the invention range in size from about 8 to about 100 nucleotide residues. In yet a more preferred embodiment, the oligonucleotide sequences range in size from about 8 to about 30 nucleotide residues. In a most preferred embodiment, the antisense sequences have 20 nucleotide residues.

However, the invention is not intended to be limited to the number of nucleotide residues in the oligonucleotide sequence disclosed herein. Any oligonucleotide sequence which is capable of reducing expression of IKKα and/or IKKα kinase domain is contemplated to be within the scope of this invention. For example, oligonucleotide sequences may range in size from about 3 nucleotide residues to the entire IKKα and/or IKKα kinase domain cDNA sequence. The art skilled know that the degree of sequence uniqueness decreases with decreasing length, thereby reducing the specificity of the oligonucleotide for IKKα mRNA and/or IKKα kinase domain mRNA.

The antisense oligonucleotide sequences which are useful in the methods of the instant invention may comprise naturally occurring nucleotide residues as well as nucleotide analogs. Nucleotide analogs may include, for example, nucleotide residues which contain altered sugar moieties, altered inter-sugar linkages (e.g., substitution of the phosphodiester bonds of the oligonucleotide with sulfur-containing bonds, phosphorothioate bonds, alkyl phosphorothioate bonds, N-alkyl phosphoramidates, phosphorodithioates, alkyl phosphonates and short chain alkyl or cycloalkyl structures), or altered base units. Oligonucleotide analogs are desirable, for example, to increase the stability of the antisense oligonucleotide compositions under biologic conditions since natural phosphodiester bonds are not resistant to nuclease hydrolysis. Oligonucleotide analogs may also be desirable to improve incorporation efficiency of the oligonucleotides into liposomes, to enhance the ability of the compositions to penetrate into the cells where the nucleic acid sequence whose activity is to be modulated is located, in order to reduce the amount of antisense oligonucleotide needed for a therapeutic effect thereby also reducing the cost and possible side effects of treatment.

Antisense oligonucleotide sequences may be synthesized using any of a number of methods known in the art, as well as using commercially available services (e.g., Genta, Inc.). Synthesis of antisense oligonucleotides may be performed, for example, using a solid support and commercially available DNA synthesizers. Alternatively, antisense oligonucleotides may also be synthesized using standard phosphoramidate chemistry techniques. For example, it is known in the art that for the generation of phosphodiester linkages, the oxidation is mediated via iodine, while for the synthesis of phosphorothioates, the oxidation is mediated with 3H-1,2-benzodithiole-3-one,1-dioxide in acetonitrile for the step-wise thioation of the phosphite linkages. The thioation step is followed by a capping step, cleavage from the solid support, and purification on HPLC, e.g., on a PRP-1 column and gradient of acetonitrile in triethylammonium acetate, pH 7.0.

b. Ribozyme

In some alternative embodiments, the agent that specifically reduces IKKα kinase activity is a ribozyme. Ribozyme sequences have been successfully used to inhibit the expression of several genes including the gene encoding VCAM1, which is one of the integrin α4β1 ligands [U.S. Pat. No. 6,252,043, incorporated in its entirety by reference].

The term “ribozyme” refers to an RNA sequence that hybridizes to a complementary sequence in a substrate RNA and cleaves the substrate RNA in a sequence specific manner at a substrate cleavage site. Typically, a ribozyme contains a “catalytic region” flanked by two “binding regions.” The ribozyme binding regions hybridize to the substrate RNA, while the catalytic region cleaves the substrate RNA at a “substrate cleavage site” to yield a “cleaved RNA product.” The nucleotide sequence of the ribozyme binding regions may be completely complementary or partially complementary to the substrate RNA sequence with which the ribozyme binding regions hybridize. Complete complementarity is preferred, in order to increase the specificity, as well as the turnover rate (i.e., the rate of release of the ribozyme from the cleaved RNA product), of the ribozyme. Partial complementarity, while less preferred, may be used to design a ribozyme binding region containing more than about 10 nucleotides. While contemplated to be within the scope of the claimed invention, partial complementarity is generally less preferred than complete complementarity since a binding region having partial complementarity to a substrate RNA exhibits reduced specificity and turnover rate of the ribozyme when compared to the specificity and turnover rate of a ribozyme which contains a binding region having complete complementarity to the substrate RNA. A ribozyme may hybridize to a partially or completely complementary DNA sequence but cannot cleave the hybridized DNA sequence since ribozyme cleavage requires a 2′-OH on the target molecule, which is not available on DNA sequences.

The ability of a ribozyme to cleave at a substrate cleavage site may readily be determined using methods known in the art. These methods include, but are not limited to, the detection (e.g., by Northern blot analysis as described herein, reverse-transcription polymerase chain reaction (RT-PCR), in situ hybridization and the like) of reduced in vitro or in vivo levels of RNA which contains a ribozyme substrate cleavage site for which the ribozyme is specific, compared to the level of RNA in controls (e.g., in the absence of ribozyme, or in the presence of a ribozyme sequence which contains a mutation in one or both unpaired nucleotide sequences which renders the ribozyme incapable of cleaving a substrate RNA).

Ribozymes contemplated to be within the scope of this invention include, but are not restricted to, hammerhead ribozymes [See e.g., Reddy et al., U.S. Pat. No. 5,246,921; Taira et al., U.S. Pat. No. 5,500,357, Goldberg et al., U.S. Pat. No. 5,225,347, the contents of each of which are herein incorporated by reference], Group I intron ribozyme [Kruger et al. (1982) Cell 31: 147-157], ribonuclease P [Guerrier-Takada et al. (1983) Cell 35: 849-857], hairpin ribozyme [Hampel et al., U.S. Pat. No. 5,527,895 incorporated by reference], and hepatitis delta virus ribozyme [Wu et al. (1989) Science 243:652-655].

A ribozyme may be designed to cleave at a substrate cleavage site in any substrate RNA so long as the substrate RNA contains one or more substrate cleavage sequences, and the sequences flanking the substrate cleavage site are known. In effect, expression in vivo of such ribozymes and the resulting cleavage of RNA transcripts of a gene of interest reduces or ablates expression of the corresponding gene.

For example, where the ribozyme is a hammerhead ribozyme, the basic principle of a hammerhead ribozyme design involves selection of a region in the substrate RNA which contains a substrate cleavage sequence, creation of two stretches of antisense oligonucleotides (i.e., the binding regions) which hybridize to sequences flanking the substrate cleavage sequence, and placing a sequence which forms a hammerhead catalytic region between the two binding regions.

In order to select a region in the substrate RNA which contains candidate substrate cleavage sites, the sequence of the substrate RNA needs to be determined. The sequence of RNA encoded by a genomic sequence of interest is readily determined using methods known in the art. For example, the sequence of an RNA transcript may be arrived at either manually, or using available computer programs (e.g., GENEWORKS, from IntelliGenetic Inc., or RNADRAW available from the internet at ole@mango.mef.ki.se), by changing the T in the DNA sequence encoding the RNA transcript to a U.

Substrate cleavage sequences in the target RNA may by located by searching the RNA sequence using available computer programs. For example, where the ribozyme is a hammerhead ribozyme, it is known in the art that the catalytic region of the hammerhead ribozyme cleaves only at a substrate cleavage site which contains a NUH, where N is any nucleotide, U is a uridine, and H is a cytosine (C), uridine (U), or adenine (A) but not a guanine (G). The U-H doublet in the NUH cleavage site does not include a U-G doublet since a G would pair with the adjacent C in the ribozyme and prevent ribozyme cleavage. Typically, N is a G and H is a C. Consequently, GUC has been found to be the most efficient substrate cleavage site for hammerhead ribozymes, although ribozyme cleavage at CUC is also efficient.

In a preferred embodiment, the substrate cleavage sequence is located in a loop structure or in an unpaired region of the substrate RNA. Computer programs for thc prediction of RNA secondary structure formation are known in the art and include, for example, “RNADRAW” [ole@mango.mef.ki.se], “RNAFOLD” [Hofacker et al. (1994) Monatshefte F. Chemie 125:167-188; McCaskill (1990) Biopolymers 29:1105-1119]. “DNASIS” (Hitachi), and The Vienna Package.

In addition to the desirability of selecting substrate cleavage sequences which are located in a loop structure or an unpaired region of the substrate RNA, it is also desirable, though not required, that the substrate cleavage sequence be located downstream (i.e., at the 3′-end) of the translation start codon (AUG or GUG) such that the translated truncated polypeptide is not biologically functional.

In a preferred embodiment, the ribozyme is an “IKKα kinase domain ribozyme” which refers to a ribozyme whose substrate cleavage sequence is designed to partially or completely hybridize with at least a portion of the IKKα kinase domain mRNA. An “IKKαt kinase domain ribozyme” also refers to a ribozyme that has at least 51%, preferably at least 70%, more preferably at least 80%, and most preferably 100% homology to at least a portion of the IKKα kinase domain mRNA.

In another embodiment, the ribozyme is an “IKKα ribozyme” which refers to a ribozyme whose substrate cleavage sequence is designed to partially or completely hybridize with at least a portion of the IKKα mRNA. An “IKKα ribozyme” also refers to a ribozyme that has at least 51%, preferably at least 70%, more preferably at least 80%, and most preferably 100% homology to at least a portion of the IKKα mRNA.

It is known in the art that the specificity of ribozyme cleavage for a substrate RNA molecule is determined by the sequence of nucleotides which flank the substrate cleavage site and which hybridize with the ribozyme binding regions. Thus, ribozymes can be designed to cleave at different locations within a substrate RNA molecule by altering the sequence of the binding regions that surround the ribozyme catalytic region of the ribozyme such that the binding regions hybridize with any known sequence on the substrate RNA.

In addition to varying the sequence of the binding regions to effect binding to different locations on the RNA substrate, the number of nucleotides in each of the ribozyme binding regions may also be altered in order to change the specificity of the ribozyme for a given location on the RNA substrate. The number of nucleotides in a binding region is preferably between about 5 and about 25 nucleotides, more preferably between about 11 and about 15 nucleotides, yet more preferably between about 7 nucleotides and about 10 nucleotides.

One of skill in the art appreciates that it is not necessary that the two binding regions which flank the ribozyme catalytic region be of equal length. Binding regions which contain any number of nucleotides are contemplated to be within the scope of this invention so long as the desirable specificity of the ribozyme for the RNA substrate and the desirable cleavage rate of the RNA substrate are achieved. One of skill in the art knows that binding regions of longer nucleotide sequence, while increasing the specificity for a particular substrate RNA sequence, may reduce the ability of the ribozyme to dissociate from the substrate RNA following cleavage to bind with another substrate RNA molecule, thus reducing the rate of cleavage. On the other hand, though binding regions with shorter nucleotide sequences may have a higher rate of dissociation and cleavage, specificity for a substrate cleavage site may be compromised.

It is well within the skill of the art to determine an optimal length for the binding regions of a ribozyme such that a desirable specificity and rate of cleavage are achieved. Both the specificity of a ribozyme for a substrate RNA and the rate of cleavage of a substrate RNA by a ribozyme may be determined by, for example, kinetic studies in combination with Northern blot analysis or nuclease protection assays.

In a preferred embodiment, the complementarity between the ribozyme binding regions and the substrate RNA is complete. However, the invention is not limited to ribozyme sequences in which the binding regions show complete complementarity with the substrate RNA. Complementarity may be partial, so long as the desired specificity of the ribozyme for a substrate cleavage site, and the rate of cleavage of the substrate RNA are achieved. Thus, base changes may be made in one or both of the ribozyme binding regions as long as substantial base pairing with the substrate RNA in the regions flanking the substrate cleavage sequence is maintained and base pairing with the substrate cleavage sequence is minimized. The term “substantial base pairing” means that greater than about 65%, more preferably greater than about 75%, and yet more preferably greater than about 90% of the bases of the hybridized sequences are base-paired.

It may be desirable to increase the intracellular stability of ribozymes expressed by an expression vector. This is achieved by designing the expressed ribozyme such that it contains a secondary structure (e.g., stem-loop structures) within the ribozyme molecule. Secondary structures which are suitable for stabilizing ribozymes include, but are not limited to, stem-loop structures formed by intra-strand base pairs. An alternative to the use of a stem-loop structure to protect ribozymes against ribonuclease degradation is by the insertion of a stem loop at each end of the ribozyme sequence [Sioud and Drlica (1991) Proc. Natl. Acad. Sci. USA 88:7303-7307]. Other secondary structures which are useful in reducing the susceptibility of a ribozyme to ribonuclease degradation include hairpin, bulge loop, interior loop, multibranched loop, and pseudoknot structure as described in “Molecular and Cellular Biology,” Stephen L. Wolfe (Ed.), Wadsworth Publishing Company (1993) p. 575. Additionally, circularization of the ribozyme molecule protects against ribonuclease degradation since exonuclease degradation is initiated at either the 5′-end or 3′-end of the RNA. Methods of expressing a circularized RNA are known in the art [see, e.g., Puttaraju et al. (1993) Nucl. Acids Res. 21:4253-4258].

Once a ribozyme with desirable binding regions, a catalytic region and nuclease stability has been designed, the ribozyme may be produced by any known means including chemical synthesis. Chemically synthesized ribozymes may be introduced into a cell by, for example, microinjection electroporation, lipofection, etc. In a preferred embodiment, ribozymes are produced by expression from an expression vector which contains a gene encoding the designed ribozyme sequence.

3. Other Agents

While the present invention is illustrated herein using antibody and nucleic acid sequences that specifically reduce IKKα kinase activity, the invention expressly contemplates within its scope other agents (e.g., organic molecules, inorganic molecules, etc.) so long as the agent is capable of specifically reducing IKKα kinase activity. In a preferred embodiment, the agent reduces IKKα kinase activity by acting either competitively or non-competitively. Such agents may be identified by screening libraries of test compounds using a competitive binding assay. In a competitive binding assay, IKKα kinase domain or IKKα is contacted with a labelled known IKKα kinase domain ligand (e.g., anti-IKKαkinase domain antibody), or IKKα ligand (e.g., anti-IKKα antibody). The test compounds are tested for their ability to inhibit binding of the labelled ligand to IKKα kinase domain or to IKKα. Compounds which inhibit such binding are identified as agents which are capable of inhibiting the specific binding of IKKα kinase domain or IKKα to the ligand, and thus as candidate compounds useful in the invention's methods.

IKKα kinase domain ligands and/or IKKα ligands that specifically reduce IKKα kinase activity other than those described above (e.g., antibodies) are also contemplated to be within the scope of the invention. These ligands may be determined using routine methods available to those skilled in the art. For example, antibodies against IKKα kinase domain or IKKα make possible methods for isolating other IKKα kinase domain ligands or IKKα ligands. One method takes advantage of an antibody characteristic known as idiotypy. Each antibody contains a unique region that is specific for an antigen. This region is called the idiotype. Antibodies themselves contain antigenic determinants; the idiotype of an antibody is an antigenic determinant unique to that molecule. By immunizing an organism with antibodies, one can raise “anti-antibodies” that recognize antibodies, including antibodies that recognize the idiotype. Antibodies that recognize the idiotype of another antibody are called anti-idiotypic antibodies. Some anti-idiotypic antibodies mimic the shape of the original antigen that the antibody recognizes and are said to bear the “internal image” of the antigen [Kennedy (1986) Sci. Am. 255:48-56]. For example, anti-idiotypic antibodies have been successfully generated against anti-ELAM1 antibodies and were found to recognize the ELAM1 ligand [U.S. Pat. No. 6,252,043, incorporated in its entirety by reference].

Other molecules which find use as agents for specifically reducing IKKα kinase activity include naturally occurring compounds and man-made compounds such as those in combinatorial libraries, which can be screened using methods of the invention, and which may be prepared using methods known in the art. These are exemplified by methods for preparing oligonucleotide libraries [Gold et al., U.S. Pat. No. 5,270,163, incorporated by reference]; peptide libraries [Koivunen et al. J. Cell Biol., 124: 373-380 (1994)]; peptidomimetic libraries [Blondelle et al., Trends Anal. Chem. 14:83-92 (1995)] oligosaccharide libraries [York et al., Carb. Res. 285:99-128 (1996); Liang et al., Science 274:1520-1522 (1996); and Ding et al., Adv. Expt. Med. Biol. 376:261-269 (1995)]; lipoprotein libraries [de Kruif et al., FEBS Lett., 399:232-236 (1996)]; glycoprotein or glycolipid libraries [Karaoglu et al., J. Cell Biol. 130:567-577 (1995)]; or chemical libraries containing, for example, drugs or other pharmaceutical agents [Gordon et al., J. Med. Chem. 37:1385-1401 (1994); Ecker and Crook, Bio/Technology 13:351-360 (1995), U.S. Pat. No. 5,760,029, incorporated by reference]. Libraries of diverse molecules also can be obtained from commercial sources.

ii. Carriers

Compositions that are useful in the instant invention may comprise a carrier that is linked to an agent that specifically reduces IKKα: kinase activity, and is optionally also linked to one or more reporter molecule, chelator, cytotoxin, and therapeutic nuclide. The terms “carrier” and “backbone” as used herein refer to a molecule which is capable of forming a covalent and/or non-covalent linkage with an agent that specifically reduces IKKα kinase activity, and which is capable of delivering the agent to a cell and/or tissue. Exemplary carriers include, but are not limited to, dextrans as described in U.S. Pat. No. 6,409,990, the entire contents of which are incorporated herein by reference; liposomes, including crosslinked liposomes as described in, for example, Hood et. al., Science 296, 2402-2405 (2002); polyethylene glycol based conjugates as described in, for example, Lee, et. al., Bioconjug Chem 10:973-81(1999); acrylic acid based conjugates as described in, for example, Liu, et. al., Biomacromolecules 2:362-8 (2001); acrylamide and N-(2-hydroxypropyl) methacrylamide (HPMA) polymers as described in, for example, Luo, Pharm Res 2002 April;19(4):396-402; colloidal gold conjugates as described in, for example, Gole, et. al., Bioconjug Chem 12:684-90 (2001); genetically or chemically modified viruses such as adenovirus, herpes simplex virus and others such as those described in Nicklin, et. al, Mol Ther 4:534-42(2001); quantum dots (for example, zinc sulfide-capped cadmium selenide) as described in, for example, Chan et. al., Science 281:2016-2018(1998); inorganic crystals, preferably phosphors, with a particle size of 0.1-0.3 microns, stabilized in suspension with polycarboxylic acids such as those described in Beverloo, et. al. Anal Biochem 203:326-34(1992); fluorescent nanoparticles (9)(10)(11); plasmon resonant particles (e.g., colloidal silver) as described in, for example, Schulz, et. al., PNAS 97, 996-1001; polystyrene based particles as described in, for example, Härmä et.al., Clin Chem 47:561-568 (2001). In one preferred embodiment, the carrier is dextran. The term “dextran” as used herein includes unbranched dextran as well as dextran which has been modified as described in, for example, U.S. Pat. No. 6,409,990 issued on Jun. 25, 2002 to Vera, the entire contents of which are incorporated herein by reference. In preferred embodiments, dextran contains leash structures which are accomplished through reaction of an allyl group with aminoethanethiol. When dextran is used, it may be selected from any molecular weight appropriate for the ultimate use of the molecule, its leashes, and conjugates. Different diagnostic and therapeutic applications will call for different molecular weight dextrans, as the person of ordinary skill in the art is aware.

In most advantageous embodiments, it is preferred that at least one chemical group be conjugated to the dextran backbone via the amino groups of the leashes. These chemical groups may be selected from any of a variety of compounds having useful therapeutic or diagnostic uses, including but not limited to: chelators, receptor ligands, lectins, enzymatic substrates, nucleic acids, peptides, polysaccharides, monosaccharides, radiosensitizers, radioprotectors, and dyes. The groups need not be directly useful, but may be indirectly useful by permitting targeting to a given cell or tissue type such that another functional moiety attached to the backbone may perform the affirmative or negative function desired.

Methods for linking a dextran carrier to the agents of the invention, reporter molecules, cytotoxins and nucleotide sequences are known in the art, such as those described in U.S. Pat. No. 6,409,990, the entire contents of which are incorporated by reference.

The terms “link,” “conjugate,” “attach” and grammatical equivalents thereof when used in reference to a carrier molecule and another molecule refer to create a covalent and/or non-covalent bond between the carrier molecule and other molecule.

Methods for chemically linking a carrier molecule to an agent of the invention, reporter molecule, chelator, cytotoxin, therapeutic nuclides, and nucleotide sequence are known in the art. For example, methods for conjugating polysaccharides to peptides are exemplified by, but not limited to coupling via alpha- or epsilon-amino groups to NaIO₄-activated oligosaccharide, using squaric acid diester (1,2-diethoxycyclobutene-3,4-dione) as a coupling reagent, coupling via a peptide linker wherein the polysaccharide has a reducing terminal and is free of carboxyl groups (U.S. Pat. No. 5,342,770), coupling with a synthetic peptide carrier derived from human heat shock protein hsp65 (U.S. Pat. No. 5,736,146), and using the methods of U.S. Pat. No. 4,639,512. Methods for conjugating proteins to proteins include coupling with a synthetic peptide carrier derived from human heat shock protein hsp65 (U.S. Pat. No. 5,736,146), the methods used to conjugate peptides to antibodies (U.S. Pat. Nos. 5,194,254; 4,950,480), the methods used to conjugate peptides to insulin fragments (U.S. Pat. No. 5,442,043), the methods of U.S. Pat. No. 4,639,512, and the method of conjugating the cyclic decapeptide polymyxin B antibiotic to and IgG carrier using EDAC [1-ethyl-3-(3-dimethylaminopropyl)carbodiimide]-mediated armide formation. (See e.g., Drabick et al., Antimicrob. Agents Chemother., 42:583-588 [1998]). Approaches to conjugate nucleic acids to proteins are also known in the art, such as those described in U.S. Pat. Nos. 5,574,142; 6,117,631; and 6,110,687; each of is incorporated in its entirety by reference. Methods for conjugating lipids to peptides have been described in the art including, but not limited to, the use of reductive amination and an ether linkage which contains a secondary or tertiary amine (U.S. Pat. No. 6,071,532), the methods of U.S. Pat. No. 4,639,512, the methods used for covalently coupling peptides to unilamellar liposomes (Friede et al., Vaccine, 12:791-797 [1994]), of coupling human serum albumin to liposomes using the hetero-bifunctional reagent N-succinimidyl-S-acetylthioacetate (SATA) (Kamps et al., Biochim. Biophys. Acta, 1278:183-190 [1996]), of coupling antibody Fab′ fragments to liposomes using a phospholipid-poly(ethylene glycol)-maleimide anchor (Shahinian et al., Biochim. Biophys. Acta, 1239:157-167 [1995]), and of coupling Plasmodium CTL epitope to palmitic acid via cysteine-serine spacer amino acids (Verheul et al., J. Immunol. Methods, 182:219-226 [1995]).

The invention's compositions which contain a carrier molecule which specifically binds to IKKα kinase domain and/or IKKα can be detected in an individual using an in vivo imaging method, such as radionuclide imaging, positron emission tomography, computerized axial tomography, X-ray or magnetic resonance imaging method, or can be detected using an ex vivo method, wherein, following administration, a sample of the tissue is obtained from the individual, and specific binding of the agent in the sample is detected (e.g., by immunohistochemical analysis).

The invention's compositions which contain a carrier moleucle which specifically binds to an IKKα kinase domain and/or IKKα can be detected directly by detecting the agent, or indirectly by detecting the presence of a moiety such as by detecting radioactivity emitted by a radionuclide moiety. Specifically bound agent also can be detected indirectly by further contacting it with a reagent that specifically interacts with the agent, or with a moiety linked to the agent, and detecting interaction of the reagent with the agent or label. For example, the moiety can be detected by contacting it with an antibody that specifically binds the moiety, particularly w-hen the moiety is linked to the agent. The moiety also can be, for example, a substrate, which is contacted by an enzyme that interacts with and changes the moiety such that its presence can be detected. Such indirect detection systems, which include the use of enzymes such as alkaline phosphatase, horseradish peroxidase, beta-galactosidase and the like, are well known in the art and commercially available, as are the methods for incorporating or, linking the particular moiety to a particular type of agent.

iii. Cytotoxins and Radionuclides

The agents of the invention that specifically reduce IKKα kinase activity may desirably comprise one or more cytotoxins, therapeutic radionuclides, and/or phototherapy compounds. The term “cytotoxin” as used herein refers to any substance having a toxic effect upon cells, including (for example) “tumor chemotherapeutic compounds,” i.e., compounds that delaying the onset of development of tumor development and/or reduce the number, weight, volume, and/or growth rate of tumors. Cytotoxins are exemplified by, without limitation, second messengers such as cAMP,; Bacterial toxins such as the exemplary Pertussis toxin, Cholera toxin, and C3 exoenzyme; Lectins such as Ricin A (Engert et al. Blood. 1997 Jan. 15;89(2):403-10.). Also included are chemotherapeutic agents exemplified by Topisomerase inhibitors such as etoposide, Campothecin irinotecan, topotecan, anthracyclines (doxorubicine, daunorubicine); Microtubule inhibitors such as vincristine, vinblastine, vinorelbine, paclitaxel, docetaxel; Platinum containing compounds such as cisplatin, carboplatin, oxaloplatin, etc.; Alkylating agents such as cyclophosphamide, and ifosfamide; Antimetabolites such as methotrexate and mercaptoprine; Anti-estrogens such as tamoxifen and toremifene; Retinoids such as all trans-retinoic acid; and others such as Adriamycin, gemcitabine, and 5-fluoruracil (Cancer: Principles and Practice of Oncology (1997) Ed. DeVita, Hellman and Rosenberg Lippincott-Raven Publishers, Philadelphia pp375-498).

Also included within the scope of the invention are cytotoxins such as Maytanisoids (Liu et al. (1996) Proc Natl Acad Sci USA. 93:8618-23.)

The invention's agents may further include therapeutic radionuclides. These are exemplified by Yttrium 90 (Hendrix et al. (2002) J Oncol Nurs. 6:144-8); Bismuth 213 (Sandmaier et al. (2002) Blood. 100:318-26); and Astatine 211 (Kennel et al. (2002) At. Radiation Res. 157:633-41)

The invention's agents may further include phototherapy compounds, such as, without limitation, propenochlorine and benzochlorine (Chen et al. (2002) Cancer J 8:154-63).

iv. Chelators and Imaging Reporter Molecules

The invention's agents that specifically reduce IKKα kinase activity may comprise one or more chelators and/or imaging reporter molecules such as those described in U.S. Pat. No. 6,409,990, the entire contents of which are incorporated by reference.

The term “chelator” refers to a molecule that is capable of reacting with another molecule to form a chelate (i.e., cyclic structure) that usually (but not necessarily) contains 5 or 6 atoms in a ring. Preferably, the ring has a central metal ion (such as bivalent copper or bivalent or trivalent iron) that is held in a coordinating complex by one or more groups (as citrate or ethylenediamine) each of which can attach itself to the central ion by at least two bonds. Exemplary chelators include, without limitation, tetraazacyclododecanetetraacetic acid (DOTA) [Sieving et al. (1990) Bioconjugate Chem 1: 65-71], mercaptoacetylglycylglycyl-glycine (MAG3) [Fritzberg et al. (1986) J Nucl Med 27: 111-116], diethylenetriamine pentaacetic acid (DTPA) [Krejcarek et al. (1977) Biochem Biophys Res Comm 77: 581-585], 4-[2-[(2-mercapto-2-methylpropyl)methylamino]-ethyl]-6,6-dimethyl-2-thiomorpholinone (DADT) [Baidoo et al. (1990) Bioconjugate Chem 1: 132-137], and Deferoxamine [Yokoyama et al. (1982) J Nucl Med 23:909-914.].

The terms “reporter” and “reporter molecule” refer to a molecule that is detectable in any detection system, including, but not limited to enzyme (e.g., ELISA, as well as enzyme-based histochemical assays), fluorescent, radioactive, and luminescent systems. Examples of imaging reporters include, without limitation, Technetium-99m (Tc-99m), Indium-111 (In-III), Gallium-68 (Ga-68), Gallium-67 (Ga-67), Rhenium-186 (Re-186) [Visser et al. (1993) J Nucl Med 34: 1953-1963], Rhenium-1 88 (Re-1 88) [Guhlke et al. (1998) Nucl Med Biol 25: 621-631]; Iodine-123 (1-123), Iodine-125 (1-125), Iodine-131 (1-131), Iodine (I), Gadolinium (Gd), Ytterbium (Yb) [Krause et al. (1996) Invest Radiol 31:502-511], Dysprosium (Dy) [Vera et al. (2002) Acad Radiol 9:784-792], Europium (Eu), Perflubron-based emulsions [Mattrey et al. (1990) Invest Radiol 25: 915-921], and Microbubble-based emulsions [Sirlin et al. (1999) Ultrasound Med Biol 25: 331-338].

M. Reducing Mammary Epithelial Cell Proliferation and Mammary Tumors In A Subject

With respect to administration of the agents of the invention to a subject, it is contemplated that the agents be administered in a “pharmaceutically effective amount.” The terms “pharmaceutically effective amount,” “therapeutically effective amount,” and “therapeutic amount” are used interchangeably herein to refer to an amount which is sufficient to achieve a desired result. In particular, a therapeutic amount is that amount that results in reducing a mammary tumor in the treated subject. The term “reducing a mammary tumor” and grammatical equivalents as used herein refer to delaying the onset of mammary tumor development and/or reducing the number, weight, volume, and/or growth rate of mammary tumors. Preferably, though not necessarily, “reducing a mammary tumor” refers to complete elimination of the onset of mammary tumor development, and/or complete elimination of an increase in weight, volume, and/or growth rate of mammary tumors.

One of ordinary skill recognizes that a therapeutically effective amount varies depending on the therapeutic agent used, the subject's age, condition, and the extent of the disease in the subject. Generally, the dosage should not be so large as to cause adverse side effects, such as hyperviscosity syndromes, pulmonary edema, congestive heart failure, and the like. The dosage can also be adjusted by the individual physician or veterinarian to achieve the desired therapeutic goal.

As used herein, the actual amount encompassed by the term “pharmaceutically effective amount” will depend on the route of administration, the type of subject being treated, and the physical characteristics of the specific subject under consideration. These factors and their relationship to determining this amount are well known to skilled practitioners in the medical, veterinary, and other related arts. This amount and the method of administration can be tailored to achieve optimal efficacy but will depend on such factors as weight, diet, concurrent medication and other factors which those skilled in the art will recognize.

The dosage amount and frequency are selected to create an effective level of the agent without substantially harmful effects. When administered orally or intravenously, the dosage of a agent will generally range from 0.001 to 1000 mg/Kg/day, more preferably from 0.01 to 100 mg/Kg/day, and most preferably from 0.1 to 10 mg/Kg/day. To achieve these concentrations in the subject, when intended for oral administration, the weight of the agent may be from 0.01% to 90%, more preferably from 0.1% to 50%, and most preferably from 0.1% to 70% of the total weight of the composition. Preferred parenteral dosage units contain from 0.001% to 10%, more preferably from 0.01% to 10%, and most preferably from 0.01% to 1% by weight of any one of the agents.

A pharmaceutically effective amount may be determined using in vitro assays employing normal mammary epithelial cell lines (for example, HC11), tumor mammary epithelial cell lines (for example, human tumor mammary epithelial cell lines such as MCF7, T47D, MDA-MB-231, MDA-MB-435, SKBR3, BT474, and Hs578T; and rodent tumor mammary epithelial cell lines such as mouse CSMLO, mouse N1H-3T3, and rat RM22-F5), primary cultured normal mammary epithelial cells, and/or primary cultured tumor mammary epithelial cell lines. Methods for the isolation and culture of normal and tumor mammary epithelial cells are known in the art [Pullan and Streuli (1997) supra] and disclosed herein. Alternatively, pharmaceutically effective amounts of the invention's agents may be determined using in vivo assays (e.g., animal models) known in the art.

Methods of administering a pharmaceutically effective amount of the invention's agents are well known in the art and include, without limitation, administration in parenteral, oral, intraperitoneal, and sublingual routes. Parenteral routes of administration include, for example, subcutaneous, intravenous, intramuscular, intrastemal injection, and infusion routes. In a preferred embodiment, the agent is administered into the mammary tumor.

The agents of the invention may be administered before, concomitantly with, and/or after manifestation of one or more symptoms of mammary tumors. The term “concomitant” when in reference to the relationship between administration of a compound and a disease symptoms means that administration occurs at the same time as, or during, manifestation of the disease symptom. Also, the invention's agents may be administered before, concomitantly with, and/or after administration of another type of drug or therapeutic procedure (e.g., surgery, chemotherapy, radiotherapy, etc.).

Pharmaceutical compositions preferably comprise one or more agents of the present invention associated with one or more pharmaceutically acceptable carrier, diluent or excipient. In preparing such compositions, the active ingredients are usually mixed with or diluted by an excipient or enclosed within such a carrier which can be in the form of a capsule or sachet in which the coating may be gelatin, sugar, shellac, and other enteric coating agents. When the excipient serves as a diluent, it may be a solid, semi-solid, or liquid material which acts as a vehicle, carrier, or medium for the active ingredient. Thus, the compositions can be in the form of tablets, pills, powders, elixirs, suspensions, emulsions, solutions, syrups, soft and hard gelatin capsules, suppositories, sterile injectable solutions and sterile packaged powders. Examples of suitable excipients include lactose, dextrose, sucrose, sorbitol, mannitol, starch, gum acacia, calcium silicate, microcrystalline cellulose, polyvinylpyrrolidone, cellulose, water, syrup, and methyl cellulose.

Pharmaceutically acceptable carriers are known in the art such as those described in, for example, Remingtons Pharmaceutical Sciences, Mack Publishing Co. (A. R. Gennaro edit. 1985). Exemplary pharmaceutically acceptable carriers are sterile saline, phosphate-buffered saline at physiological pH, polyethylene glycols, polypropylene copolymCers, and water solublc gels. Other agents that may be included with the invention's compositions include, for example, diluents, fillers, salts, buffers, preservatives (e.g., sodium benzoate, sorbic acid and esters of p-hydroxybenzoic acid), stabilizers, dyes, antioxidants, flavoring agents, lubricating agents (such as talc, magnesium stearate and mineral oil), wetting agents, emulsifying and suspending agents, preserving agents such as methyl- and propylhydroxybenzoates, sweetening agents and/or flavoring agents.

The pharmaceutically acceptable carriers may be liquid, with the compositions being, for example, an oral syrup or injectable liquid. Compositions in solid or liquid form may include an agent which binds to the active component(s) and thereby assists in the delivery of the active components. Suitable agents which may act in this capacity include a monoclonal or polyclonal antibody, a protein or a liposome.

When intended for oral administration, the composition is preferably in either solid or liquid form, where semi-solid, semi-liquid, suspension and gel forms are included within the forms considered herein as either solid or liquid.

As a solid composition for oral administration, the composition may be formulated into a powder, granule, compressed tablet, pill, capsule, chewing gum, wafer or the like form. Such a solid composition will typically contain one or more inert diluents or edible carriers. In addition, one or more of the following adjuvants may be present: binders such as carboxymethylcellulose, ethyl cellulose, microcrystalline cellulose, or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin, a flavoring agent such as peppermint, methyl salicylate or orange flavoring, and a coloring agent.

In one preferred embodiment, the subject to whom the agents are administered is “capable of developing a mammary tumor” in a tissue. This term encompasses any mammal that contains mammary epithelial cells, including (for example), mammals that are predisposed to developing mammary epithelial tumors for genetic or environmental reasons.

N. Detecting Tumor Mammary Epithelial Cells

The invention further provides methods for detecting tumor mammary epithelial cells in a sample, by detecting increased IKKα kinase activity in the tumor mammary epithelial cells or in the sample compared to IKKα kinase activity in normal mammary epithelial cells or in a control sample. The term “increased IKKα kinase activity” when in reference to the activity of a first sample relative to a second sample, means that the level of IKKα kinase activity in the first sample is higher than in the second sample by any amount that is statistically significant using any art-accepted statistical method of analysis. In a preferred embodiment, increased IKKα kinase activity in a first sample refers to a quantity of IKKα kinase activity in the first sample that is preferably at least 10% greater than, more preferably at least 50% greater than, yet more preferably at least 75% greater than, even more preferably at least 90% greater than, the quantity in a second sample.

In a further embodiment, the detection relies, in part, on detecting an increased level of phosphorylation of IKKα or of the IKKα kinase domain since it is the inventors' view that activated IKKα and activated IKKα kinase domain are phosphorylated at Serine, whereas inactivated IKKα and inactivated IKKα kinase domain are not so phosphorylated. The terms “activated IKKα” and “activated IKKα kinase domain” refer to an IKKα polypeptide and an IKKα kinase domain polypeptide, respectively, that have IKKα kinase activity. This is in contrast to the terms “inactivated IKKα” and “inactivated IKKα kinase domain” which refer to an IKKα polypeptide and to an IKKα kinase domain polypeptide, respectively, that do not have IKKα kinase activity.

Accordingly, the invention provides a method for detecting tumor mammary epithelial cells in a sample, comprising: a) providing: i) a sample suspected of comprising tumor mammary epithelial cells; and ii) an anti-phosphoserine antibody; b) administering the antibody to the sample such that the antibody binds to phosphoserine in an TKKα kinase domain to produce a treated sample; and c) detecting increased binding of the antibody to the phosphoserine in the treated sample compared to binding of the antibody to the phosphoserine in a control sample, thereby detecting tumor mammary epithelial cells in the sample.

The term “increased binding of antibody to phosphoserine,” and grammatical equivalents, refers to an increased level of binding of the antibody to phosphoserine by any amount that is statistically significant using any art-accepted statistical method of analysis. In a preferred embodiment, an increased level of binding of an antibody to phosphoserine in a sample refers to a quantity of binding of the antibody to phosphoserine that is preferably at least 10% more than, more preferably at least 50% more than, yet more preferably at least 75% more than, even more preferably at least 90% more than, the quantity in another sample (such as a control sample). The level of binding of an antibody to phosphoserine may be determined using methods known in the art, and such binding is detectable in any detection system, including, but not limited to enzyme [e.g., enzyme-linked immunosorbent assay (ELISA)], fluorescent, radioactive, and luminescent systems. It is not intended that the present invention be limited to any particular detection system or label. The tumor mammary epithelial cells may be in vitro or in vivo.

In an alternative embodiment, tumor mammary epithelial cells may be detected in a sample by detecting the level of at least a portion of a molecule selected from IKKα polypeptide, IKKα kinase domain polypeptide, IKKα mRNA, and IKKαC kinase domain mRNA, rather than by detecting IKKα kinase activity. In this embodiment, an agent that specifically binds to a molecule selected from at least a portion of a molecule selected from IKKα polypeptide, IKKα kinase domain polypeptide, IKKα mPNA, and IKKα kinase domain mRNA, is administered to the sample (e.g., biopsy) such that the agent specifically binds to the molecule, followed by detecting increased specific binding of the molecule to the agent in the treated sample or in the treated cells as compared to specific binding of the molecule to the agent in a control sample (for example, not containing tumor mammary epithelial cells). For example, the presence of elevated levels of at least a portion of IKKα polypeptide or of IKKα kinase domain polypeptide in a tissue may be detected using an anti-IKKα antibody or anti-IKKα kinase domain antibody in Western blot analysis or immunofluorescence. Alternatively, elevated levels of at least a portion of IKKα mRNA or IKKα kinase domain mRNA may be detecting using reverse transcription polymerase chain (RT-PCR), or in situ hybridization. These methods are well known and well within the ordinary skill of those in the art.

In one embodiment, the agent which is used in detecting the presence of at least a portion of a molecule selected from IKKα polypeptide, IKKαkinase domain polypeptide, IKKα mRNA, and IKKα kinase domain mRNA, can be detectably labeled, for example, by linking the agent to a moiety, which is selected based, for example, on whether specific binding of the agent is to be detected in vivo or whether a tissue to which the agent is suspected of binding is to be removed (e.g., by biopsy) and examined ex vivo.

A moiety useful for labeling an agent antagonist can be a radionuclide, a paramagnetic material, an X-ray attenuating material, a fluorescent, chemiluminescent or luminescent molecule, a molecule such as biotin, or a molecule that can be visualized upon reaction with a particular reagent, for example, a substrate for an enzyme or an epitope for an antibody. The moiety can be linked to an agent using well known methods, which are selected, in part, based on the chemical nature of the agent and the moiety. For example, where the moiety is an amino acid sequence such as a hexahistidine (His6) sequence, and the agent is a peptide, the His6 sequence can be synthesized as part of the peptide, and the His6-labeled agent can be identified by the binding of a nickel ion reagent to the His6 moiety.

A specifically bound agent can be detected in an individual using an in vivo imaging method, such as a radionuclide imaging, positron emission tomography, computerized axial tomography, X-ray or magnetic resonance imaging method, or can be detected using an ex vivo method, wherein, following administration, a sample of the tissue is obtained from the individual, and specific binding of the agent in the sample is detected (e.g., by immunohistochemical analysis).

An agent that is specifically bound to at least a portion of a molecule selected from IKKα polypeptide, IKKα kinase domain polypeptide, IKKα mRNA, and IKKα kinase domain mRNA, in a sample can be detected directly by detecting the agent, or indirectly by detecting the presence of a moiety such as by detecting radioactivity emitted by a radionuclide moiety. Specifically bound agent also can be detected indirectly by further contacting it with a reagent that specifically interacts with the agent, or with a moiety linked to the agent, and detecting interaction of the reagent with the agent or label. For example, the moiety can be detected by contacting it with an antibody that specifically binds the moiety, particularly when the moiety is linked to the agent. The moiety also can be, for example, a substrate, which is contacted by an enzyme that interacts with and changes the moiety such that its presence can be detected. Such indirect detection systems, which include the use of enzymes such as alkaline phosphatase, horseradish peroxidase, beta-galactosidase and the like, are well known in the art and commercially available, as are the methods for incorporating or, linking the particular moiety to a particular type of agent.

O. Screening Compounds

The invention further provides methods for screening and identifying test compounds which are capable of reducing mammary epithelial cell proliferation and/or mammary tumors. A screening assay of the invention can be performed by administering one or more test compounds to mammary epithelial cells and detecting a reduction in IKKα kinase activity in the treated mammary epithelial cells, thereby identifying the test compound as reducing mammary epithelial cell proliferation and/or mammary tumors. Mammary epithelial cells may be contacted with the agent in vivo or ex vivo (see, for example, U.S. Pat. No. 5,622,699, incorporated by reference). Where a screening method of the invention is performed using an in vitro format, it can be adapted to automated procedure, thus allowing high throughput screening assays for examining libraries of molecules to identify inhibitors of mammary epithelial cell proliferation. The mammary epithelial cells used in the assays may be a normal cell line (e.g., HC11), a tumor cell line (e.g., human tumor mammary epithelial cell lines such as MCF7, T47D, MDA-MB-231, MDA-MB-435, SKBR3, BT474, and Hs578T, and a rodent tumor mammary epithelial cell line such as mouse CSMLO and NIH-3T3, and rat RM22-F5), or primary cultured normal or tumor cells.

In another embodiment, the invention provides a method for screening compounds that reduce mammary epithelial cell proliferation by screening those compounds that impact IKKα kinase activity. For example, the invention provides a method for screening a test compound as reducing mammary epithelial cell proliferation, comprising: a) providing: i) a sample comprising IKKα or IKKαkinase domain; ii) an IKKαsubstrate, iii) a test compound; and b) contacting the sample, the IKKαsubstrate, and the test compound such that the IKKαsubstrate is phosphorylated by the IKKα or the IKKα kinase domain; and c) detecting a reduction in the level of phosphorylation of the IKKα substrate in the presence of the test compound compared to in the absence of the test compound, thereby identifying the test compound as reducing mammary epithelial cell proliferation.

The terms “IKKαsubstrate” and “IKKαkinase substrate” are used interchangeably to refer to any protein that is capable of being phosphorylated by the IKKαkinase activity of IKKα. Such substrates are known in the art and are exemplified by IκBα and by κB2 (also known as p100).

The term “reduction in the level of phosphorylation of an IKKα substrate,” and grammatical equivalents, refers to reducing the level of phosphorylation of the IKKα substrate by any amount that is statistically significant using any art-accepted statistical method of analysis. In a preferred embodiment, a reduced level of phosphorylation of an IKKα substrate in a sample refers to a quantity of phosphorylation of the IKKα substrate that is preferably at least 10% less than, more preferably at least 50% less than, yet more preferably at least 75% less than, even more preferably at least 90% less than, the quantity in another sample (such as a control sample), and most preferably is at the same level which is observed in the other sample to which it is being compared (such as a control sample). A reduced level of phosphorylation of an IKKα substrate need not, although it may, mean an absolute absence of phosphorylation of the IKKα substrate. The invention does not require, and is not limited to, methods that wholly eliminate phosphorylation of the IKKα substrate.

The level of phosphorylation of the IKKα substrate may be determined using methods known in the art [DiDonato et al. (1997) supra], and disclosed herein, for determining kinase activity. For example, a cell extract is treated with IKKγ antibody and/or IKKα antibody for immunoprecipitation of the IKK complex that contains IKKα. The immunoprecipitated complex is further incubated (in the absence and presence of the test compound) with the IKKα substrate (IκBα or and by κB2). The reaction mixture is resolved by SDS-polyacrylamide gel electrophoresis, and the level of phosphorylation of the substrate on the gel is determined (e.g., by autoradiography). In order to more specifically measure the effect on the kinase activity of IKKα rather than of another kinase, in a preferred embodiment, the IKKα in the sample is recombinant IKKα. In a preferred embodiment, the recombinant IKKα has the amino acid sequence of SEQ ID NO:13 (FIG. 10) or of SEQ ID NO:15 (FIG. 11).

In a further embodiment, the invention provides a method for screening compounds that reduce mammary epithelial cell proliferation by screening those compounds that impact alter the level of phosphorylated IKKα, and/or phosphorylated IKKα kinase domain. In other words, this method relies, in part, on the inventors' observation that IKKα that has kinase activity is itself phosphorylated at a Serine residue, whereas IKKα that does not have kinase activity is not so phosphorylated. Thus, the invention provides a method for screening a test compound as reducing mammary epithelial cell proliferation, comprising: a) providing: i) a sample comprising IKKα or IKKα kinase domain; ii) an anti-phosphoserine antibody; iii) a test compound; and b) contacting the sample, the antibody, and the test compound such that the antibody binds to the IKKα or IKKα kinase domain; and c) detecting a reduction in the level of binding of the antibody to the IKKα or the IKKα kinase domain in the presence of the test compound compared to in the absence of the test compound, thereby identifying the test compound as reducing mammary epithelial cell proliferation.

The terms “reduction in the level of binding of an antibody to IKKα or IKKα kinase domain,” and grammatical equivalents, refers to reducing the level of binding of the antibody to IKKα or IKKα kinase domain by any amount that is statistically significant using any art-accepted statistical method of analysis. In a preferred embodiment, a reduced level of binding of an antibody to IKKα or IKKα kinase domain in a sample refers to a quantity of binding of the antibody to IKKα or IKKα kinase domain that is preferably at least 10% less than, more preferably at least 50% less than, yet more preferably at least 75% less than, even more preferably at least 90% less than, the quantity in another sample (such as a control sample), and most preferably is at the same level that is observed in the other sample to which it is being compared (such as a control sample). A reduced level of binding of an antibody to IKKα or IKKα kinase domain need not, although it may, mean an absolute absence of binding of the antibody to IKKα or IKKα kinase domain. The invention does not require, and is not limited to, methods that wholly eliminate binding of the antibody to IKKα or IKKα kinase domain. The level of binding of an antibody to IKKα or IKKα kinase domain may be determined using methods known in the art, and is detectable in any detection system, including, but not limited to enzyme [e.g., enzyme-linked immunosorbent assay (ELISA)], fluorescent, radioactive, and luminescent systems. It is not intended that the present invention be limited to any particular detection system or label.

The invention also provides methods for screening test compounds that specifically reduce IKKα kinase activity using transgenic animals, and observing the impact of the test compounds on mammary tumor formation. In one embodiment, the invention provides a method for screening a test compound as specifically reducing IKKα kinase activity, comprising: a) providing: i) a first transgenic non-human animal selected from mouse, sheep, pig, rabbit and cattle expressing: 1) an oncogene that causes mammary tumors in said first transgenic animal; ii) a second transgenic non-human animal expressing: 1) said oncogene; and 2) a moduiied IKKα polypeptide having reduced IKKα kinase activity, wherein said second transgenic animal is of the same species as said first transgenic animal; and iii) a test compound; and b) administering said test compound to said first transgenic animal to produce a first treated transgenic animal and to said second transgenic animal to produce a second treated transgenic animal; and c) detecting a reduction in mammary tumors in said first treated transgenic animal that is not greater than a reduction in mammary tumors in said second treated transgenic animal, thereby identifying said test compound as specifically reducing IKKα kinase activity.

The term “not greater than” when in refernce to the reduction in mammary tumors in a first animal compared to a second animal means that the delay in the onset, and/or the reduction in size, weight, level of angiogenesis, or any other indicia of mammary tumor development, in the first animal is not greater than in the second animal by any amount that is statistically significant using any art-accepted statistical method of analysis.

In cotrast, the term “greater reduction in mammary tumors” when in reference to the level of reduction in mammary tumors in a second animal as compared to a first animal means that the delay in the onset, and/or the reduction in size, weight, level of angiogenesis, or any other indicia of mammary tumor development, in the second animal is greater than in the first animal by any amount that is statistically significant using any art-accepted statistical method of analysis. In a preferred embodiment, greater reduction in mammary tumors in a second animal refers to a delay in onset, or reduction in the quantity of an indicator of mammary tumor development, that is preferably at least 10% more than, more preferably at least 50% more than, yet more preferably at least 75% more than, even more preferably at least 90% more than, the quantity of the same indicator in a first animal.

In a more preferred embodiment, the method further comprises identifying the test compound as reducing mammary epithelial cell proliferation. In an alternative embodiment, the non-human animal is a mouse, more preferably, the mouse comprises a mutation in the IKKα kinase domain, yet more preferably, the mutation in the IKKα kinase domain comprises replacing at least one of the Serine residues at amino acids 176 and 180 of IKKα kinase polypeptide with Alanine. Alternatively, the mutation in the IKKα kinase domain comprises replacing Lysine at amino acid 44 of IKKα kinase polypeptide with Alanine or Methionine. In one embodiment, the oncogene is selected from neu (also known as c-erbB-2, or HER2/neu), c-myc, and s-src, and more preferably, the oncogene is the neu oncogene. Data provided herein (Example 9) shows the generation of the exemplary Ikkα^{AA/AA}/MMTV-c-neu animals.

Methods for preparing libraries of molecules, which can be screened using the methods of the invention to identify test compounds that reduce mammary epithelial cell proliferation and/or mammary tumors are known in the art. [Gold et al., U.S. Pat. No. 5,270,163, incorporated by reference]; peptide libraries [Koivunen et al. J. Cell Biol., 124: 373-380 (1994)]; peptidomimetic libraries [Blondelle et al., Trends Anal. Chem. 14:83-92 (1995)] oligosaccharide libraries [York et al., Carb. Res. 285:99-128 (1996); Liang et al., Science 274:1520-1522 (1996); and Ding et al., Adv. Expt. Med. Biol. 376:261-269 (1995)]; lipoprotein libraries [de Kruif et al., FEBS Lett., 399:232-236 (1996)]; glycoprotein or glycolipid libraries [Karaoglu et al., J. Cell Biol. 130:567-577 (1995)]; or chemical libraries containing, for example, drugs or other pharmaceutical agents [Gordon et al., J. Med. Chem. 37:1385-1401 (1994); Ecker and Crook, Bio/Technology 13:351-360 (1995), U.S. Pat. No. 5,760,029, incorporated by reference]. Libraries of diverse molecules also can be obtained from commercial sources. Furthermore, test compounds which are capable of reducing mammary epithelial cell proliferation and/or mammary tumors may be identified by direct screening in vitro and in vivo of natural products and combinatorial libraries.

P. Transgenic Animals

The present invention provides transgenic non-human animals which express a nucleotide sequence of interest in an age-related manner. These animals provide useful models for disease (e.g., breast cancer), and for the study of normal phenomena, such as mammary epithelial cell development. For example, the invention's transgenic animals may be used as animal models for monitoring the efficacy of drug treatment of breast cancer. In one embodiment, as disclosed herein, inhibition of MMTV-c-neu induced tumors by the administration of an IKKα targeting drug in a wildtype background should not exceed what is observed in the Ikkα^{AA/AA} transgenic mice that are crossed with MMTV-c-neu transgenic mice, unless additional targets are involved.

In one embodiment, the invention provides transgenic animals expressing a modified IKKα polypeptide (exemplified by Ikkα^{AA/AA} transgenic mice) or a modified IκBα polypeptide (exemplified by MMTV-Iκbα^{A32/36} transgenic mice).

i. Transgenic Animals Expressing a Modified IKKα Polypeptide

The invention provides transgenic animals expressing a modified IKKα polypeptide that has reduced IKKα kinase activity and reduced IκBα phosphorylation activity. The terms “modified,” “mutant,” and grammatical equivalents thereof when used in reference to an amino acid sequence refer to an amino acid sequence which differs by insertion, deletion, and/or substitution (conservative or non-conservative) of one or more amino acids from the amino acid sequence to which it is compared. The term “conservative substitution” of an amino acid refers to the replacement of that amino acid with another amino acid which has a similar hydrophobicity, polarity, and/or structure. For example, the following aliphatic amino acids with neutral side chains may be conservatively substituted one for the other: glycine, alanine, valine, leucine, isoleucine, serine, and threonine. Aromatic amino acids with neutral side chains which may be conservatively substituted one for the other include phenylalanine, tyrosine, and tryptophan. Cysteine and methionine are sulphur-containing amino acids which may be conservatively substituted one for the other. Also, asparagine may be conservatively substituted for glutamine, and vice versa, since both amino acids are amides of dicarboxylic amino acids. In addition, aspartic acid (aspartate) my be conservatively substituted for glutamic acid (glutamate) as both are acidic, charged (hydrophilic) amino acids. Also, lysine, arginine, and histidine my be conservatively substituted one for the other since each is a basic, charged (hydrophilic) amino acid. Guidance in determining which and how many amino acid residues may be substituted, inserted or deleted without abolishing biological or immunological activity may be found using computer programs well known in the art, for example, DNAStar software.

In a preferred embodiment, the transgenic animal comprises a mutation in the IKKα kinase domain. More preferably, the mutation comprises replacing Serine at each of amino acids 176 and 180 of IKKα kinase polypeptide with Alanine.

In one embodiment, the transgenic animal is chimeric. Such animals are useful for generating transgenic animals that are heterozygous for the mutation in the IKKα polypeptide. In an alternative embodiment, the transgenic animal is homozygous for the mutation, and is exemplified by the Ikkα^{AA/AA} knockin mouse disclosed herein. Preferably, the female of the transgenic animal has a reduced mammary epithelial cell proliferation rate during pregnancy, lactation, or both pregnancy and lactation compared to a control female animal. For example, data herein shows that the mammary epithelial cell proliferation rate in females of the Ikkα^{AA/AA} knockin mouse at P10 had approximately half of the wt rate, while at L1 the females had a rate of 0.9% versus 5.7% in control females, as determined by BrdU incorporation.

Preferably also, the female of the transgenic animal has a reduced number of mammary epithelial cells during pregnancy, lactation, or both pregnancy and lactation compared to a control female animal. For example, data herein demonstrates that females of the Ikkα^{AA/AA} knockin mouse at P10 had a 50% lower number of mammary epithelial cells than in the wt animals as determined by staining mammary glands with antibodies to PCNA.

In one embodiment, the female of the transgenic animal has substantially the same (i.e., in the absence of a statistically significant difference) mammary epithelial cell apoptotic rate compared to a control female animal, as determined by, for example, the TUNEL assay used herein. Date herein demonstrates that there were no significant differences in the apoptotic rates, measured by TUNEL assay, between wt and Ikkα^{AA/AA} mammary glands at either P10 or L1.

In an alternative embodiment, the female of the transgenic animal has a reduced level of cyclin D1 polypeptide, cyclin D1 mRNA, or both cyclin D1 polypeptide and cyclin D1 mRNA compared to a control female animal. For example, data herein shows that at P6 or P10, there was reduction of approximately 50%″ in mammary glands of Ikkα^{AA/AA} knockin mice as determined by immunoblot analysis (FIG. 4B). Also, only 20% of the Ikkα^{AA/AA} mammary epithelial cells were cyclin D1 positive, as compared to 42.4% of the wt cells (FIG. 4A).

In another embodiment, the female transgenic animal has a reduced level (i.e., at least 5% less, more preferably at least 10% less, more preferably, at least 50% less, and most preferably, at least 90% less than a control) of NF-κB activation during pregnancy, lactation, or both pregnancy and lactation compared to a control female animal. For example, data herein shows that between P6 and L1, there was an almost complete inhibition of NF-κB activation in Ikkα^{AA/AA} mammary glands, as determined by gel mobility shift assay (FIG. 5A). In one preferred embodiment, the female of the animal has normal epidermis and limbs compared to a control female animal, as determined by clinical, and/or histological observation.

In an alternative preferred embodiment, the female of the transgenic animal has the same number of T cells and/or does not exhibit osteoporosis compared to a control female animal.

ii. Transgenic Animals Expressing a Modified IκBα Polypeptide

The invention further provides a transgenic animal expressing a modified IκBα polypeptide, wherein the female of the transgenic animal has a reduced level of IκBα phosphorylation during pregnancy, lactation, or both pregnancy and lactation, compared to a control animal. In one preferred embodiment, the modified IκBα polypeptide contains a mutation in which Serine at each of amino acids 32 and 36 is replaced with Alanine. As disclosed herein, this mutation of IκBα renders it insensitive to IKK activation.

In one embodiment, the female of the transgenic animal has a reduced mammary epithelial cell proliferation rate during pregnancy, lactation, or both pregnancy and lactation compared to a control female animal. For example, data herein demonstrates a lobuloalveolar developmental defect by wholemount analysis of mammary glands. In another embodiment, the female of the transgenic animal has a reduced level of cyclin D1 polypeptide, cyclin D1 mRNA, or both cyclin D1 polypeptide and cyclin D1 mRNA compared to a control female animal. This is exemplified by the instantly disclosed data showing that at L1, there was reduced expression of cyclin D1 as determined by immunoblot analysis (FIG. 7C).

iii. Generation of Transgenic Animals

Transgenic animals of the invention may be generated by introducing a construct that contains the nucleic acid sequences of interest into target cells. Several methods are available for accomplishing this, including microinjection, retroviral infection, and implantation of embryonic stem cells. These methods are discussed as follows.

1. Microinjection Methods in Mice, Sheep, Pigs, Rabbits and Cattle

Direct microinjection of expression vectors into pronuclei of fertilized eggs is the preferred, and most prevalent, technique for introducing heterologous nucleic acid sequences into the germ line. Technical aspects of the microinjection procedure and important parameters for optimizing integration of nucleic acid sequences have been previously described [Hogan et al., (1986) Manipulation of the Mouse Embryo: A Laboratory Manual. Cold Spring Harbor, N.Y.: Cold Spring Harbor Lab.].

Once the expression vector has been injected into the fertilized egg cell, the cell is implanted into the uterus of a pseudopregnant female and allowed to develop into an animal. Of the founder transgenic animals born, 70% carry the expression vector sequence in all of their cells, including the germ cells. The remaining 30% of the transgenic animals are chimeric in somatic and germ cells because integration of the expression vector sequence occurs after one or more rounds of replication. Heterozygous and homozygous animals can then be produced by interbreeding founder transgenics. This method has been successful in producing transgenic mice, sheep, pigs, rabbits and cattle [Hammer et al., (1986) J. Animal Sci.:63:269; Hammer et al., (1985) Nature 315:680-683].

2. Retroviral Methods

Retroviral infection of pre-implantation embryos with genetically engineered retroviruses may also be used to introduce transgenes into an animal cell. For example, blastomeres have been used as targets for retroviral infection [Jaenisch, (1976) Proc. Natl. Acad. Sci USA 73:1260-1264]. Transfection is typically achieved using a replication-defective retrovirus carrying the transgene [Jahner et al., (1985) Proc. Natl. Acad. Sci. USA 82:6927-6931; Van der Putten et al., (1985) Proc. Natl. Acad Sci USA 82:6148-6152]. Transfection is obtained, for example, by culturing eight-cell embryos, from which the zona pellucida has been removed with fibroblasts which produce the virus [Van der Putten (1985), supra; Stewart et al., (1987) EMBO J. 6:383-388]. The transfected embryos are then transferred to foster mothers for continued development. Alternatively, infection can be performed at a later stage. Virus or virus-producing cells can be injected into the blastocoele [Jahner et al., (1982) Nature 298:623-628]. Yet another alternative method involves intrauterine retroviral infection of the midgestation embryos [Jahner et al. (1982), supra].

The advantages of retroviral infection methods include the ease of transfection and the insertion of a single copy of the transgene, which is flanked by the retroviral long terminal repeats (LTRs), into the chromosome. However, this method is not a preferred method because most of the founders will show mosaicism since infection occurs after cell division has begun. This necessitates outbreeding to establish homozygous and heterozygous lines suitable for analysis of gene expression. More importantly, the retroviral LTR sequences may interfere with the activity of the hINV upstream sequences in directing expression of the heterologous nucleic aid sequences.

3. Embryonic Stem Cell Implantation

Another method of introducing transgenes into the germ line involves using embryonic stem (ES) cells as recipients of the expression vector. ES cells are pluripotent cells directly derived from the inner cell mass of blastocysts [Doetchman et al., (1988) Dev. Biol. 127:224-227], from inner cell masses [Tokunaga et al., (1989) Jpn. J. Anim. Reprod. 35:173-178], from disaggregated morulae [Eistetter, (1989) Dev. Gro. Differ. 31:275-282] or from primordial germ cells [Matsui et al., (1992) Cell 70:841-847]. Expression vectors can be introduced into ES cells using any method which is suitable for gene transfer into cells, e.g., by transfection, cell fusion, electroporation, microinjection, DNA viruses, and RNA viruses [Johnson et al., (1989) Fetal Ther. 4 (Suppl. 1):28-39].

The advantages of using ES cells include their ability to form permanent cell lines in vitro, thus providing an unlimited source of genetic material. Additionally ES cells are the most pluripotent cultured animal cells known. For example, when ES cells are injected into an intact blastocyst cavity or under the zona pellucida, at the morula stage embryo, ES cells are capable of contributing to all somatic tissues including the germ line in the resulting chimeras.

Once the expression vector has been introduced into an ES cell, the modified ES cell is then introduced back into the embryonic environment for expression and subsequent transmission to progeny animals. The most commonly used method is the injection of several ES cells into the blastocoel cavity of intact blastocysts [Bradley et al., (1984) Nature 309:225-256]. Alternatively, a clump of ES cells may be sandwiched between two eight-cell embryos [Bradley et al., (1987) in “Teratocarcinomas and Embryonic Stem Cells: A Practical Approach,” Ed. Robertson E. J. (IRL, Oxford, U.K.), pp. 113-151; Nagy et al., (1990) Development 110:815-821]. Both methods result in germ line transmission at high frequency.

Target cells which contain the heterologous nucleic acid sequences are recovered, and the presence of the heterologous nucleic acid sequence in the target cells as well as in the animal is accomplished as described supra.

4. Ikkα^{AA} Mutations and Carcinogenesis.

Our preliminary data show that the Ikkα^{AA} mutation reduces tumor incidence, delays tumor onset and decreases tumor growth by the Neu/ErbB2/Her2 oncogene but not by the Ha-Ras oncogene. We have crossed Ikkα^{AA/AA} mice with mice carrying MMTV-c-Neu or MMTV-v-Ha-Ras transgenes. Ikkα^{+/+}/MMTV-c-Neu (or MMTV-v-Ha-Ras) and Ikkα^{AA/AA}/MMTV-c-Neu (or MMTV-v-Ha-Ras) littermates were observed for breast cancer formation by biweekly palpation. In over one year of observation, all of the Ikkα^{+/+}/MMTV-c-Neu females have developed tumors, while 20% of the Ikkα^{AA/AA}/MMTV-c-Neu females remained tumor free. The onset of tumor development was delayed in the Ikkα^{AA/AA}/MMTV-c-Neu mice: it took 42 weeks for 50% of these females to develop tumors compared to 29 weeks for the wt group. Tumor number was markedly decreased in the Ikkα^{AA/AA}/MMTV-c-Neu group, averaging 1.0 tumors per mouse vs 4.6 tumors per mouse for the wild type. We also noticed that the growth rate of some tumors is decreased in the Ikkα^{AA/AA} background. This effect of the Ikkα^{AA} mutation is specific to Neu/ErbB2-induced tumors as it exerted no inhibitory effect on MMTV-v-Ha-Ras induced tumors. Without limiting the mechanisms of the invention, these results suggest that IKKα-dependent NF-κB pathway is one of the signaling pathways that are activated by the Neu/ErbB2 oncogene and are required for its optimal carcinogenic activity. By contrast, the IKKα-dependent NF-κB pathway plays no role in the Ha-Ras induced mammary carcinogenesis.

Our preliminary results also show that the Ikkα^{AA} mutation reduces tumor incidence and delays tumor onset in a chemical carcinogenesis model. The fifth generation of a backcross of Ikkα^{AA} mice to the BALB/c background was used for this experiment. Six weeks old wt and Ikkα^{AA/AA} mice were given subcutaneous slow-release pellet of MPA (medroxyprogesterone acetate) followed by oral gavage of DMBA (7, 12-dimethylbenzaanthracene). Mammary tumor formation was monitored twice a week after the last injection of DMBA. Again, we found that Ikkα^{AA} mutation delays tumor onset and reduces tumor incidence. In the wt group, tumors started to appear after 5 weeks and 50% of mice had developed tumors by 7 weeks, while no tumors were detected before 10 weeks in Ikkα^{AA/AA} mice and it took 13.5 weeks for 50% of these mice to develop tumors. Tumor numbers were also decreased in the Ikkα^{AA/AA} mice, averaging 1.1 tumors per mouse vs 3.1 tumors per mouse for the wt. These results suggest that IKKα is one of the signaling pathways involved in chemically-induced mammary carcinogenesis.

We have also established an in vivo system to test the efficacy of such drugs in mice. We have injected cultured mammary tumorigenic cells into the #4 mammary glands of Rag1^{-/-} mice bilaterally at 10⁴ cell/ml×10 ml in PBS to and found that tumors can be detected around day 10 at such inoculation. Tumor growth rates will be determined by measuring the diameter of the tumors as a function of time. The Alzet osmotic pumps can be used to deliver drugs to mice at a constant infusion rate.

As described above, we have used the Ikkα^{AA/AA} mouse strain to critically evaluate the role of IKKα-driven mammary tumorigenesis in vivo, and show that IKKα plays a critical role in both Her2/ErbB2- and chemical-induced mammary gland carcinogenesis in mice. Importantly, the Ikkα^{AA} mutation was initially found to cause a specific mammary gland developmental defect. The Ikkα^{AA/AA} mice do not show profound immune deficiency that is associated with systemic inhibition of NF-kB activity through the IKKb-dependent classical pathway. They do not exhibit the defects in brain and retina development associated with loss of cyclin D1 expression either. The only defect other than mammary gland development or carcinogenesis in Ikkα^{AA/AA} mice is aberrant development of secondary lymphoid organs, affecting certain B cell-mediated responses. Since innate immunity provided by IKKb as well as T cell functions are still intact in these animals, they are healthy and are not prone to infection. Therefore, selective inhibition of IKKα activity provide a highly specific way to block the growth of Her2/ErbB2 positive breast tumors and sensitize them to apoptosis with a minimal amount of side effects. The studies herein lay the foundation to a new kind of therapeutic strategy which specifically targets a basic mechanism involved in Her2/ErbB2-dependent breast carcinogenesis.

In one embodiment for screening compounds, we will set up a cellular screening assay using immortalized mouse mammary epithelial cells in which IKKα-dependent NF-κP activation can be easily measured. Using these cells we will screen compound libraries for IKK inhibitors. Candidate compounds will be examined in mice for their effect on the development of ErbB2-induced mammary carcinomas. Once verified to be functional in mice with the desired pharmacodynamic properties, candidate compounds will be tested for their activity on xenograft models of human breast cancer, and if effective, will be subjected to Phase I/II clinical trials.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); rpm (revolutions per minute); BSA (bovine serum albumin); CFA (complete Freund's adjuvant); IFA (incomplete Freund's adjuvant); IgG (immunoglobulin G); IM (intramuscular); IP (intraperitoneal); IV (intravenous or intravascular); SC (subcutaneous); H₂O (water); HCl (hydrochloric acid); aa (amino acid); bp (base pair); kb (kilobase pair); kD (kilodaltons); wt (wild-type); gm (grams); μg (micrograms); mg (milligrams); ng (nanograms); μl (microliters); ml (milliliters); mm (millimeters); nm (nanometers); μm (micrometer); M (molar); mM (millimolar); μM (micromolar); U (units); V (volts); MW (molecular weight); sec (seconds); min(s) (minute/minutes); hr(s) (hour/hours); MgCl₂ (magnesium chloride); NaCl (sodium chloride); OD₂₈₀ (optical density at 280 nm); OD₆₀₀ (optical density at 600 nm); BrdU (5-bromo-2′-deoxyuridine); PAGE (polyacrylamide gel electrophoresis); PBS (phosphate buffered saline [150 mM NaCl, 10 mM sodium phosphate buffer, pH 7.2]); H&E (hematoxylin and eosin); PCR (polymerase chain reaction); PEG (polyethylene glycol); PMSF (phenylmethylsulfonyl fluoride); RT-PCR (reverse transcription PCR); SDS (sodium dodecyl sulfate); Tris (tris(hydroxymethyl)aminomethane); w/v (weight to volume); v/v (volume to volume); Amersham Pharmacia (Amersham Pharmacia Biotech AB, Piscataway, N.J.); ICN (ICN Pharmaceuticals, Inc., Costa Mesa, CA); ATCC (American Type Culture Collection, Rockville, Md.); Jackson (Jackson Laboratories, Bar Harbor, Me.); Stratagene (Stratagene, La Jolla, Calif.); PharMingen (PharMingen, San Diego, Calif.); Applied Precision (Applied Precision, Issaquah, WA); NEN (NEN Life Sciences Products, Boston, Mass.); Imgenex (Imgenex, San Diego, Calif.); R & D Systems (R & D Systems, Minneapolis, Minn.); Sigma (Sigma Chemical Co., St. Louis, Mo.); Santa Cruz (Santa Cruz Biotechnology, Santa Cruz, CA); Qiagen (Qiagen, Inc., Valencia, Calif.); Vector (Vector Laboratories, Burlingame, Calif.); Zymed (Zymed Laboratories, South San Francisco, Calif.); and Silicon Graphics (Silicon Graphics, Mountain View, Calif.).

The following methods were used in the Examples described below.

Wholemount, Histology, and Immunofluorescence Analyses

Wholemount staining of mammary glands was performed as known in the art (See e.g., Seagroves et al., Genes Dev., 12:1917-1928 [1998]). For histological analysis, mammary tissues were fixed in cold 4% paraformaldehyde and embedded in paraffin, 5 μm sections were cut and stained with H&E. For immunostaining, paraffin sections were rehydrated using standard protocols and microwaved for 20 min in 10 mM sodium citrate. Sections were then incubated with biotin-labeled anti-BrdU (BD, PharMingen), biotin-labeled anti-PCNA (Zymed), or anti-cyclin D1 (Santa Cruz) antibodies followed by incubation with biotin-conjugated anti-rabbit secondary antibody. The antigen-antibody complexes were conjugated to horseradish peroxidase by the Elite ABC Kit (Vector) and visualized with Cyanine 3 using the TSA Fluorescence System (NEN). Images were captured with a DeltaVision deconvolution microscope system (Applied Precision). Data sets were deconvolved and analyzed using SoftWorx software (Applied Precision) on a Silicon Graphics Octane workstation.

IκB Kinase and Gel Shift Assays

IKK immunecomplex kinase assays were perfonred as known in the art (See e.g., DiDonato et al. [1997], supra) except that an IKKγ antibody (764, PharMingen) was used for immunoprecipitation. Electrophoretic mobility shift assays of NF-κB and NF-1 were conducted as known in the art (See e.g., Senftleben et al. [2001a,b], supra).

Northern and Western Blot Analyses

Total RNA was isolated from #4 mammary glands using RNeasy Kit (Qiagen). 20 μg samples were analyzed by Northern hybridization after gel separation using the QuikHyb Hybridization Solution (Stratagene) and ³²P labeled probes (Prime It II, Stratagene) as known in the art (See, Mori et al., EMBO J., 90:5772-5781 [2000]). Protein lysates were prepared from #4 mammary glands and analyzed by immunoblotting as known in the art (See, Hu et al. [1999], supra). The IKKα antibody used in these experiments was obtained from Imgenex.

### Example 1

### Generation of Ikkα^{AA/AA} Knockin Mice

A genomic Ikkα clone (Hu et al., supra) was used to construct the Ikk^{AA} targeting vector. A 6.0 kb BamHI fragment containing 1.3 and 4.7 kb of DNA upstream and downstream to codons 176/180, respectively, was subcloned into pUC18. A NotI site was introduced by site-directed mutagenesis into the 7^{th} intron using the QuikChange Site-Directed Mutagenesis Kit (Stratagene), followed by insertion of a NotI fragment containing a Neo^{r} cassette flanked by two LoxP sites. Serines 176 and 180 were converted to alanines using the QuikChange Site-Directed Mutagenesis Kit (Stratagene) (Primer sequence 5′-GAT GTT GAT CAA GGA GCG CTC TGT ACA GCG TTT GTG GGA ACA TTG-3′; SEQ ID NO:1; and 5′-CAA TGT TCC CAC AAA CGC TGT ACA GAG CGC TCC TTG ATC AAC ATC-3′; SEQ ID NO:2) to introduce a new Eco47III site. A 200 bp BclI-NcoI fragment covering the mutagenized region was sequenced to exclude other mutations and then swapped back into the pUC18 clone containing the 6.0 kb genomic DNA with the Neo^{r} cassette. The targeting vector was linearized by HindIII and electroporated into ES cells, using methods known in the art.

Approximately 150 G418 resistant clones were collected and screened by PCR to identify homologous integrants at the Ikka locus (the primers used were: p309: 5′-CCGGAAGCTACTCAACAAACCAG-3′ (SEQ ID NO:3); and p5′Neo: 5′-CCATCATGGCTGATGCAA-3′ (SEQ ID NO:4). Six clones positive for the Ikkα^{AA} allele were identified and confirmed by Eco47III digestion.

The positive clones were grown up and electroporated with a Cre expression vector to delete the Neo^{r} cassette. Clones lacking the Neo^{r} cassette were identified by PCR (the primers used were: p5′Neo:above (SEQ ID NO:3); and p3′Neo: 5′-GATACCGTAAAGCACGA-3′ (SEQ ID NO:5)). One such ES cell clone was injected into blastocysts from C57B1/6 females at the UCSD knockout core facility. Two male chimeras that were crossed with C57B1/6 females gave rise to heterozygous Ikkα^{+/AA} mice that were intercrossed to obtain homozygous Ikkα^{AA/AA} mutants. Primers used for genotyping were: p392 (5′-GGATCCGATATCTGCATGAAAAC (SEQ ID NO:6)), pWT (5′-CAATGTTCCCACAAAAGATGTACAGAGACT-3′; SEQ ID NO:7)), pAA (5′-CAATGTTCCCACAAA CGCTGTACAGAGCGC-3′; SEQ ID NO:8)), and p776 (5′-GGTAAATGGCTACTAAACCGAACTTCTCC-3′ (SEQ ID NO:9)).

To confirm expression of the mutant allele, total RNA was extracted from livers of 4-week old mice and subjected to RT-PCR with primers p392 and p776, followed by Eco47III digestion and sequencing, using standard methods known in the art. All Ikka^{AA} mice used were on a mixed 129/C57B1/6 genetic background, and maintained in filter-topped cages at UCSD animal facilities according to NIH guidelines.

The construction of HA-tagged human IκBα cDNA with Ser to Ala substitutions at positions 32 and 36 was conducted as known in the art and previously described (See, DiDonato et al., 1996, supra). The Iκbα^{A32/36} cDNA was inserted at the HindIII-XbaI sites of an expression vector that contains the MMTV promoter (i.e., a mammary gland specific promoter). The MMTV-Iκbα^{A32/36} transgene was then purified and resuspended at 4 μg/ml in microinjection buffer (7.5 mM Tris, 0.15 MM EDTA, pH 7.4). Prior to injection, DNA was diluted at a concentration of 1.8 μg/ml and a few picoliters of the solution were microinjected into the pronuclei of C57BL/6 fertilized oocytes. Fertilized ova for the microinjection of DNA were obtained from female mice sired with stud males. To increase the yield and quality of eggs, female mice were superovulated with gonadotrophins. Groups of 20 embryos that had been injected with the transgene were reimplanted into the oviducts of pseudopregnant female recipients. These females were mated with appropriate timing to vasectomized male mice. These animals gave birth 19-20 days after implantation. Litters were weaned at 3-4 weeks of age and transgene integration was assessed by tail tissue analysis performed as known in the art. Genotyping was performed by PCR using primers specific for the HA sequence (5′-TACCCATACGATGTTCCAGATTACGCT-3′; SEQ ID NO:10) and human Iκbα (5′-TCATAACGTCAGACGCTGGCC-3′; SEQ ID NO:11).

MMTV-cyclin D1 transgenic mice were produced as known in the art and previously described (See, Wang et al., [1994], supra).

To replace serines 176 and 180 of IKKα with alanines, a NotI site was introduced into the 7^{th} intron of Ikkα, into which a loxP-flanked Neo^{r} cassette was inserted (See, FIG. 1, Panel A). Serine to alanine substitutions were introduced by site-directed mutagenesis, which also created an Eco47III site. A targeting vector containing these alterations was electroporated into ES cells and six clones with homologous recombination at the Ikkα locus were selected. To remove the Neo^{r} cassette these ES clones were electroporated with a plasmid expressing Cre recombinase. A heterozygous Ikkα^{+/AA} ES clone was selected and injected into mouse blastocysts, yielding chimeric mice. These mice were bred to produce Ikkα^{+/AA} heterozygotes, which were intercrossed to generate homozygous Ikkα^{AA/AA} knockin mice. The F2 generation was genotyped by PCR analysis of tail DNAs using allele-specific primers and confirmed by Eco47III digestion (See, FIG. 1, Panel B). The frequency of Ikkα^{+/+}:Ikkα^{+/AA}:Ikkα^{AA/AA} mice was approximately 1:2:1, as expected based on Mendelian inheritance. RT-PCR analysis of liver RNA demonstrated expression of the Ikkα^{AA}-allele (See, FIG. 1, Panel C). Sequencing of PCR products using methods known in the art, confirmed the presence of the designed mutations. Ikkα^{AA/AA} mice were normal from birth until adulthood and largely indistinguishable from their wt or heterozygote littermates in weight, size, appearance, development, fertility and behavior. In contrast to Ikkα^{-/-} mice (Hu et al., [1999], supra), Ikkα^{AA/AA} mice showed no defects in skin or limb development.

To further examine whether IKKα kinase activity contributes to IKK or NF-κB activation by proinflammatory cytokines, embryonic fibroblasts (MEF) were treated with TNFα or IL-1. In these experiments, stock solutions of TNFα (20 μg/ml) or IL-1 (10 μg/ml) were added to DMEM at 1:1000 dilution. Cells were incubated at 37° C. with 5% CO₂ for the indicated time before harvest (See, FIG. 1, for time periods).

Ikkα^{AA/AA} MEFs did not show any defects in IKK (See, FIG. 1, Panel D) or NF-κB (See, FIG. 1, Panel E) activation. IKK and NF-κB activation also proceeded normally in Ikkα^{AA/AA} mice administered endotoxin. These data are consistent with previous results, affirming that IKKβ is the major catalytic subunit responsible for IKK and NF-κB activation by proinflammatory stimuli.

### Example 2

### IKKα is Essential for Lactation and Lobuloalveolar Development During Pregnancy

Ikkα^{AA/AA} females completed pregnancy and gave birth to pups whose size and numbers were absolutely normal. However, all pups born to Ikkα^{AA/AA} mothers died within 1-2 days, in spite of normal nursing. Examination of their stomachs revealed no milk. This phenotype was specific to Ikkα^{AA/AA} mothers and was independent of the pup genotype. When cross-fostering experiments were performed (as known in the art), no pups survived with Ikkα^{AA/AA} mothers, while pups nursed by Ikkα^{+/AA} or wt mothers survived. These results confirmed that Ikkα^{AA/AA} mothers have a specific lactation defect that caused the lethality of their offspring. This defect was exhibited even after multiple pregnancies (at least 10).

Wholemount analysis of mammary glands demonstrated that Ikkα^{AA/AA} virgin females completed normal ductal development (See, FIG. 2, Panel A). Examination on day 1 after delivery (L1), however, revealed that the lobuloalveolar tree in Ikkα^{AA/AA} mammary glands was severely undeveloped. Although some side branching and sprouting of mammary ducts were observed, their extent was comparable to that of wt mammary glands at 10 days of pregnancy (See, FIG. 2, Panel A). Histological analysis confirmed the failure of lobuloalveolar development in Ikkta mammary glands (See, FIG. 2, Panel B). The number of secretory alveoli was dramatically reduced in comparison to wt glands of same development age and the alveoli of Ikkα^{AA/AA} glands were small with small or closed lumina. In summary, the extensive lobuloalveolar development which occurs during pregnancy, is severely impaired in Ikkα^{AA/AA} mice. Notably, mammary gland development in Ikkα^{AA/AA} females appeared to be normal during sexual maturation.

Expression of Milk Proteins

To evaluate the differentiation status of Ikkα^{AA/AA} mammary glands, expression of milk protein genes, β-casein and whey acidic protein (WAP), was examined by Northern blotting using methods known in the art. The results indicated that milk protein genes were activated in Ikkα^{AA/AA} mammary glands, although at slightly lower levels relative to the wt (See, FIG. 2, Panel C), suggesting that the differentiation process is not blocked.

Role of Hormonal and Epithelial-Stromal Interaction

Proper mammary gland development requires hormonal stimulation and epithelial-stromal interaction (Hennighausen and Robinson, supra; Medina, supra). To determine whether the developmental defect of Ikkα^{AA/AA} mice was intrinsic to the mammary epithelium or due to either hormonal insufficiency or stromal cell defects, transplantation experiments were performed. Small pieces of mammary gland tissue taken from wt or Ikkα^{AA/AA} donors were implanted into cleared fat pads of 3-week old Rag1^{-/-} recipients. More particularly, mammary epithelia were isolated from 8-week old nulliparous donors and implanted into cleared fat pads of bilateral #4 mammary glands (i.e., mammary gland set number 4) of 3-week old Rag1^{-/-} hosts (Jackson Laboratories).

Mammary glands #3, #4, and #5 were taken from 14-15 day pregnant mice, and epithelial cell fractions were prepared as known in the art and previously described (See, Pullan and Streuli, supra; and Seagroves et al., supra). Cells were cultured approximately 3 days before stimulation with mTNFα (20 ng/ml) or mRANKL (R&D Products, 100 ng/ml). Nuclear extracts were prepared for gel shift assay.

These mice were mated 9 weeks later with wild-type males, and their mammary glands were analyzed shortly after delivery. The #3 mammary glands were taken from the donor for internal controls. Transplanted wt epithelia showed lobuloalveolar development similar to the internal control, while transplanted Ikkα^{AA/AA} epithelia failed to develop proper lobuloalveolar structures. Hence, the developmental defect of Ikkα^{AA/AA} mice is autonomous to the mammary epithelium.

### Example 3

### Lactation Defect in Ikkα^{AA/AA} Mice is Due to Impaired Epithelial Cell Proliferation

To determine whether the mammary development defect was the result of reduced cell proliferation or increased cell death, BrdU incorporation and TUNEL assays were performed.

Wild-type and Ikkα^{AA/AA} females at either 10 days of pregnancy (P10) or L1 were administered BrdU (IP injection of BrdU [Amersham Pharmacia #RPN 201], at 100 μl/10 g body weight). The incorporation of BrdU into DNA of these animals was detected by immunohistochemistry. The proliferation index was calculated as percentages of BrdU-positive alveolar cells per total epithelial cells (See, FIG. 3). At P10 the proliferation rate in the Ikkα^{AA/AA} mammary epithelium was approximately half of the wt rate (7.4% vs. 12.8%). At L1, the defect was even more dramatic (0.9% vs. 5.7%). However, it should be noted that the number of epithelial cells was greatly reduced in the mutant glands. Similar results were obtained by staining sections of wt and Ikkα^{AA/AA} mammary glands with biotinylated anti-PCNA monoclonal antibodies (Zymed) to proliferating cell nuclear antigen (PCNA), a marker for cells at the S phase of the cell cycle. The number of PCNA positive cells in the Ikkα^{AA/AA} mammary epithelium at P10 was 50% lower than in the wt.

On the other hand, there were no significant differences in the apoptotic rates, measured by TUNEL assay performed according to the manufacturer's instructions, between wt and Ikkα^{AA/AA} mammary glands at either P10 or L1. Very few apoptotic cells could be detected at either time point. These results strongly suggest that IKKα is essential for proliferation of the lobuloalveolar epithelium in response to pregnancy signals.

### Example 4

### Defective Cyclin D1 Induction in Ikkα^{AA/AA} Mammary Glands

Since cyclin D1 is the major G1 cyclin expressed in mammary epithelial cells and cyclin D1^{-/-} females exhibit a similar defect in mammary gland development as Ikkα^{AA/AA} females (Fantl et al., supra; Sicinski et al., supra), cyclin D1 expression in wt and Ikkα^{AA/AA} mammary glands was examined. Immunohistochemical analysis performed as known in the art of wt and Ikkα^{AA/AA} mammary glands revealed that at P10 only 20.0% of the Ikkα^{AA/AA} mammary epithelial cells were cyclin D1 positive vs 42.4% of the wt cells (See, FIG. 4, Panel A). Similar results were revealed by immunoblot analysis which showed approximately 50% reduction in cyclin D1 levels at either P6 or P10 in Ikkα^{AA/AA} mammary glands (See, FIG. 4, Panel B). In these immunoblots, polyclonal anti-cyclin D1 antibody (Santa Cruz) was used. Similar results were obtained by RNA analysis by Northern blotting (as described herein) and microarray analyses (performed at the Genechip core, UCSD), which also failed to reveal decreased expression of other cell cycle regulators. These results suggest that the proliferation defect in the Ikkα^{AA/AA} mammary epithelium is due to reduced cyclin D1 expression.

### Example 5

### Defective NF-κB Activation in Ikkα^{AA/AA} Mammary Glands

In order to determine whether the reduced expression of cyclin Dl in Ikkα^{AA/AA} mammary glands is caused by an NF-κB activation defect the induction of NF-κB DNA binding activity in wt and Ikkα^{AA/AA} mammary glands was monitored by gel mobility shift assay performed as known in the art (See, FIG. 5, Panel A). The results indicated that NF-κB activation in Ikkα^{AA/AA} mammary glands was almost completely diminished between P6 and L1. However, Western blot analysis revealed similar levels of NF-κB proteins, including RelA (p65), RelB, c-Rel, NF-κB1 p105 and p50 in wt and Ikkα^{AA} mammary glands analyzed at P10 (See, FIG. 5, Panel B). Consistent with previous results (Senftleben et al. [2001b], supra), processing of NF-κB2 p100 into mature p52 was defective in Ikkα^{AA/AA} mammary glands (See, FIG. 5, Panel B). However, the total level of NF-κB2 in mammary glands was found to be low, unlike the situation in B cells (Senftleben et al., [2001b], supra). Furthermore, no p52 could be detected even in wt NF-κB complexes, which were mostly p65:p50 heterodimers.

### Example 6

### RANKL Activates NF-κB in Wild-Type, but not Ikkα^{AA/AA} Mammary Epithelial Cells

Defective mammary gland development, as exhibited by Ikkα^{AA/AA} and cyclin D1^{-/-} females, is exhibited by female mice lacking RANKL (OPGL/TRANCE/ODF) or its receptor RANK (Fata et al., supra). RANK ligation results in NF-κB activation (Anderson et al., supra). Therefore, it was postulated that the defect in mammary gland development caused by the Ikkα^{AA/AA} mutation is due to either diminished RANKL expression or defective PRANK signaling. Thus, experiments were conducted to determine the levels of RANKL mRNA expression and function.

Northern blot analysis indicated that RANKL mRNA was actually elevated in Ikkα^{AA/AA} mammary glands between P6 and P15 (See, FIG. 6, Panel A). To examine RANK signaling in wt and Ikkα^{AA/AA} mammary epithelial cells, these cells were cultured using an established procedure (See, Pullan and Streuli, supra) and stimulated with recombinant RANKL or TNFα, used as control. While TNFα induced NF-κB DNA binding activity in both wt and Ikkα^{AA/AA} epithelial cells, RANKL induced NF-κB activity (composed of p65:p50 heterodimers) in wt, but not Ikkα^{AA/AA} cells (See, FIG. 6, Panel B). These results were obtained at least three times with different primary cultures.

### Example 7

### Inhibition of NF-κB Activity in Mammary Glands Inhibits Their Development

To assess whether the developmental defect in Ikkα^{AA/AA} mammary glands could be the direct consequence of diminished NF-κB activation, transgenic mice overexpressing the IκBα superrepressor mutant in the mammary epithelium were generated (See, FIG. 7, Panel A). These animals were produced using methods commonly used in the art.

This IκBα mutant contains alanines instead of serines 32 and 36 and therefore is no longer sensitive to IKK activation (DiDonato et al., supra). A similar construct was used to inhibit NF-κB activity in thymocytes (Hettmann et al., supra). Of two lines of MMTV-Iκbα^{A32/36} transgenic mice that were analyzed, females of line 13 exhibited a lactation defect similar to Ikkα^{AA/AA} mice, resulting in death of all newborns within 48 hours, while females of line 6 could lactate and nurse their pups. Wholemount analysis of mammary glands taken at L1 confirmed a lobuloalveolar developmental defect in line 13 but not in line 6. Although both lines were positive for MMTV-Iκbα^{A32/A36} DNA (See, FIG. 7, Panel B) expression of the transgene was considerably higher in line 13 (See, FIG. 7). Correspondingly, expression of cyclin D1 was reduced in line 13 but not in line 6 mammary glands (See, FIG. 7, Panel C).

### Example 8

### The Ikkα^{AA/AA} Mutant Phenotype is Rescued by a Cyclin D1 Transgene

The results discussed in the above Examples suggested that the defect in lobuloalveolar development in Ikkα^{AA/AA} mammary glands is the result of a failure to properly induce cyclin D1 in response to activation of RANK or other receptors. If this hypothesis is correct, restoration of cyclin D1 expression in Ikkα^{AA/AA} mammary glands should suppress the developmental defect and allow normal lactation to occur. Therefore Ikkα^{AA/AA} males were crossed with MMTV-cyclin D1 transgenic females. The latter were shown to display mammary gland hyperplasia and increased susceptibility to development of breast tumors (Wang et al., supra). Ikkα^{+/AA}MMTV-cyclin D1 heterozygotes were selected and intercrossed to generate Ikkα^{AA/AA}MMTV-cyclin D1 homozygous females which were mated and analyzed for lactation and mammary gland development. Expression of the MMTV-cyclin D1 transgene in the Ikkα^{AA/AA} genetic background was sufficient to completely suppress the lactation defect even after the first pregnancy.

Wholemount analysis of mammary glands at L1 validated these results and revealed restoration of normal lobuloalveolar development by the MMTV-cyclin D1 transgene.

Immunoblot analysis (performed as described herein) indicated that expression of the MMTV-cyclin D1 transgene in the wt background resulted in only a modest elevation in cyclin D1, whereas its expression in the Ikkα^{AA/AA} background restored cyclin D1 expression to its normal level (See, FIG. 8, Panel B). However, the MMTV-cyclin D1 transgene did not restore NF-κB activity (See, FIG. 8, Panel C). These results strongly suggest that IKKα is a crucial intermediate in a signaling pathway through which RANK activation results in cyclin D1 induction, leading to increased proliferation of mammary epithelial cells (FIG. 8). Defects in at least five components of this pathway were found to result in the same outcome.

### Example 9

### The Ikkα^{AA} Mutation Reduces Tumor Incidence by MMTV-c-neu Oncogene

This Example addresses the question whether the Ikkα^{AA} mutation protects mice against breast cancer induced by oncogenes. The previous results have revealed that the Ikkα^{AA} mutation blocks pregnancy-induced NF-κB activation and subsequent cyclin D1 induction in mammary epithelia, therefore Ikkα^{AA/AA} mice become an ideal animal model to study breast tumorigenesis associated with high NF-κB activity or cyclin D1 overexpression. As in many types of cancer, tumor cells originate from the normal cells and often retain tissue-specific pathways but in a deregulated way. The inventors hypothesized that upregulation of IKKα/NTF-κb/cyclin D1 pathway uniderlies the pathogenesis of tumor formation in a subset of breast cancer cases. Possible mechanisms include overexpression of any of these components or captivation of the pathway by certain oncogenes leading to constitutive activation.

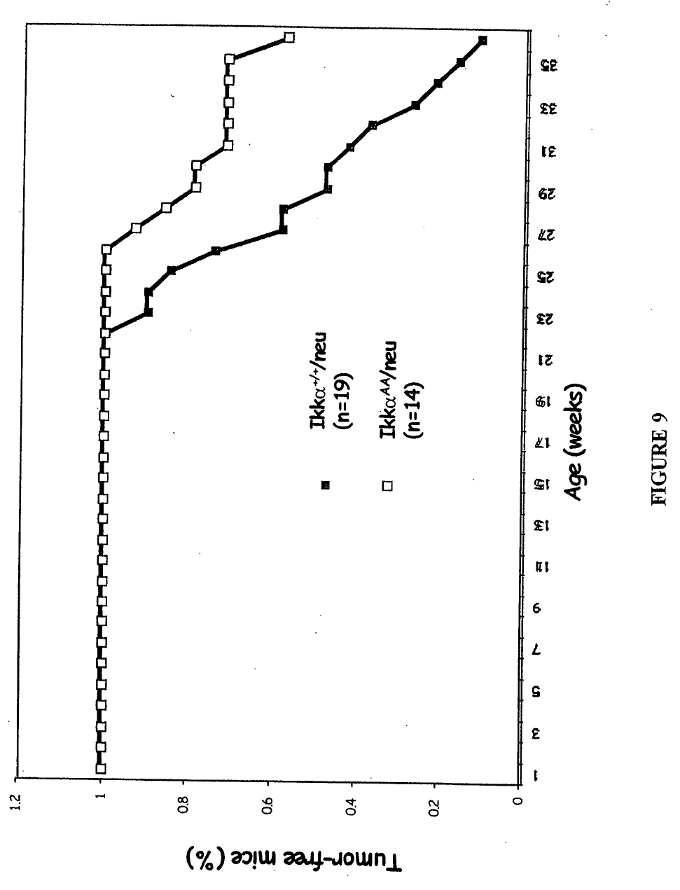

To address the question of whether Ikkα^{AA} mutation protects mice against breast cancer induced by several oncogenes, the Ikkα^{AA/AA} mice were crossed with mice carrying the MMTV-c-neu gene. Transgenic mice carrying the MMTV-c-neu are a breast cancer model which has been shown to depend on cyclin D1 to transform mammary epithelia. Littermates of Ikkα^{+/+}/MMTV-c-neu and Ikkα^{AA/AA}/MMTV-c-neu were observed for breast cancer incidence, and the results are shown in FIG. 9.

FIG. 9 shows that the Ikkα^{AA} mutation reduces tumor incidence by MMTV-c-neu oncogene. In an approximate 6- to 9-month observation period, 17 of 19 Ikkα^{+/+}/MMTV-c-neu females developed tumors, with a total of 88 tumors (average 4.6 tumor per mouse). In contrast, 6 of 14 females developed tumors in the Ikkα^{AA/AA}/MMTV-c-neu group, with a total of 14 tumors (average 1.0 tumor per mouse) (FIG. 9). Additionally, the onset of tumor development was delayed and growth rate was reduced in Ikkα^{AA/AA}/MMTV-c-neu mice compared to wt mice. These results are consistent with the inventors' previous finding that IKKα/NF-κB contribute to cyclin D1 activation in mammary glands, and this pathway is activated during tumorigenesis induced by overexpression of the neu oncogene.

### Example 10

### The Ikkα^{AA} Mutation Reduces Tumor Incidence, Delays Tumor Onset and Decreases Tumor Growth by the Neu/ErbB2/Her2 Oncogene but not the Ha-Ras Oncogene

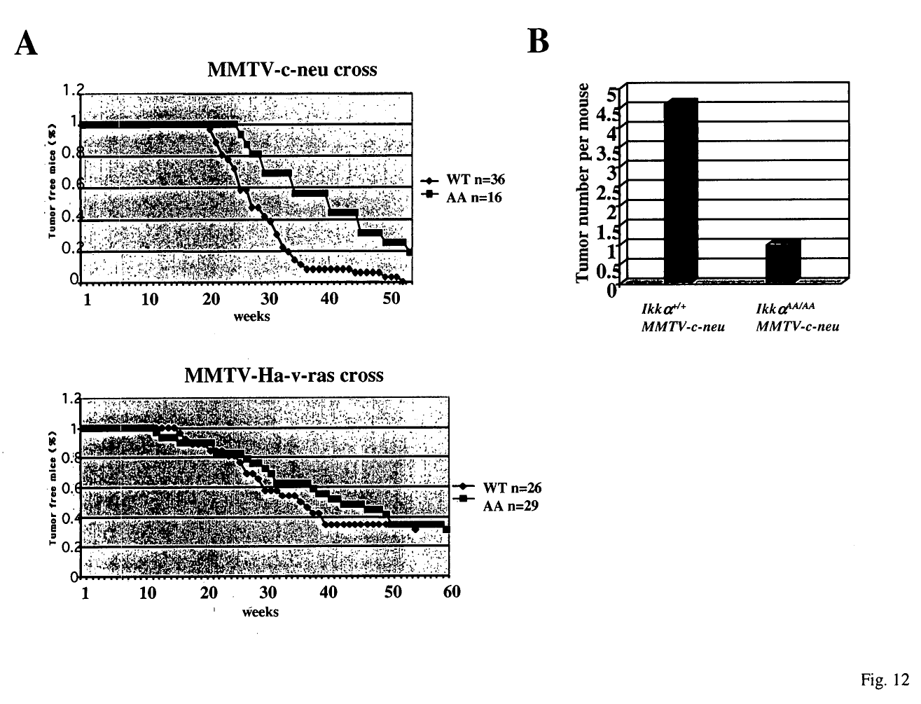

We crossed Ikkα^{AA/AA} mice with mice carrying MMTV-c-neu or MMTV-v-Ha-Ras transgenes. Ikkα^{+/+}/MMTV-c-neu (or MMTV-v-Ha-Ras) and Ikkα^{AA/AA}/MMTV-c-neu (or MMTV-v-Ha-Ras) littermates were observed for breast cancer formation by biweekly palpation. Our results indicate that homozygocity for the Ikkα^{AA} mutation reduces the incidence of mammary tumors caused by the MMTV-c-neu trans-oncogene but not the MMTV-v-Fa-Ras oncogene (FIG. 12A). in over one year of observation, all of the Ikkα^{+/+}/MMTV-c-neu females have developed tumors, while 20% of the Ikkα^{AA/AA}/MMTV-c-neu females remained tumor free. The onset of tumor development was delayed in the Ikkα^{AA/AA}/MMTV-c-Neu mice: it took 42 weeks for 50% of these females to develop tumors compared to 29 weeks for the wt group. Tumor number was markedly decreased in the Ikkα^{AA/AA}/MMTV-c-neu group, averaging 1.0 tumor per mouse vs 4.6 tumor per mouse for the wt (FIG. 12B). We also noticed that the growth rate of some tumors is decreased in the Ikkα^{AA/AA} background. This effect of the Ikkα^{AA} mutation is specific to Neu/ErbB2-induced tumors as it exerted no inhibitory effect on MMTV-v-Ha-Ras induced tumors (FIG. 12A). These results suggest that IKKα-dependent NF-κB pathway is one of the signaling pathways that are activated by the Neu/ErbB2 oncogene and are required for its optimal carcinogenic activity. By contrast, the IKKα-dependent NF-κb pathway plays no role in the Ha-Ras induced mammary carcinogenesis.

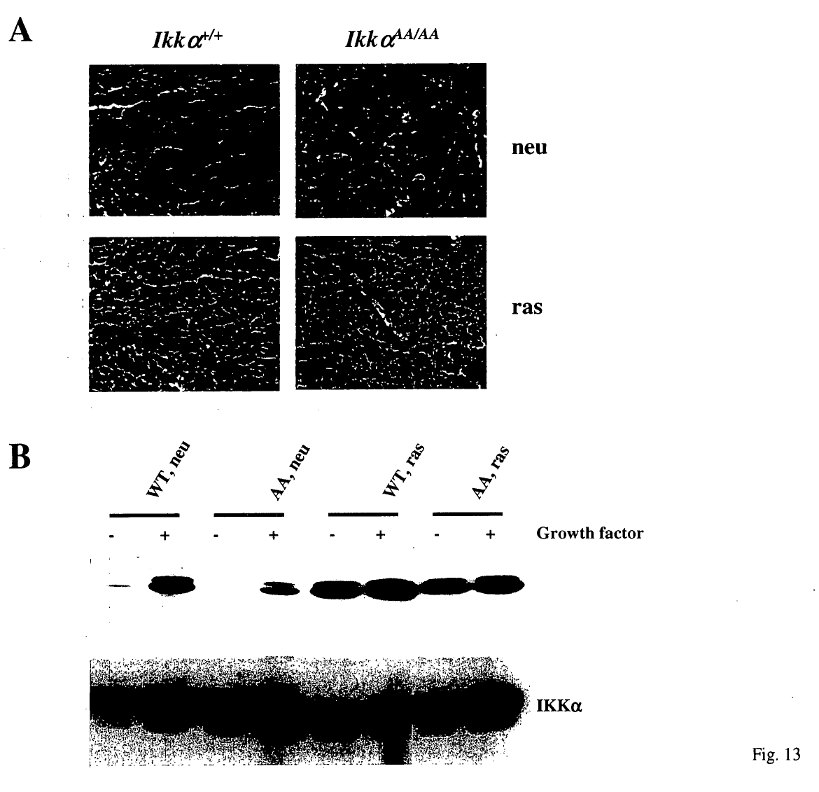

The histology of tumors isolated from Ikkα^{AA/AA}/MMTV-c-Neu mice appears to be identical to tumors from the Ikkα^{+/+}/MMTV-c-Neu mice (FIG. 13A). In agreement with published results we found higher levels of NF-κB DNA binding activity in tumor tissue relative to the surrounding normal tissue. However, no significant differences in NF-κB DNA binding activity were detected between tumors in the wt vs Ikkα^{AA/AA} backgrounds. These results suggest that once a mammary carcinoma does emerge in the Ikkα^{AA/AA} background, it relies on an IKKα-independent pathway, most likely IKKb, to activate NF-κB.

We have also cultured primary tumor cells to study their biochemical properties. Previously we found that primary mammary epithelial cells derived from Ikkα^{AA/AA} mice exhibit little difference from wt counterparts in activation of NF-κB and cyclin D1 expression when grown in rich medium. We have therefore eliminated serum and exogenous EGF from the culture medium used in these studies, and found that Ikkα^{AA/AA}/MMTV-c-Neu cells exhibited less efficient induction of cyclin D1 in response to growth factor addback relative to Ikkα^{+/+}/MMTV-c-neu cells (FIG. 13B). Interestingly, growth factor depletion and addback had no effect on cyclin D1 expression in Ha-Ras-induced tumor cells of either genotype (FIG. 13B).

### Example 11

### The Ikkα^{AA} Mutation Reduces Tumor Incidence and Delays Tumor Onset in a Chemical Carcinogenesis Model

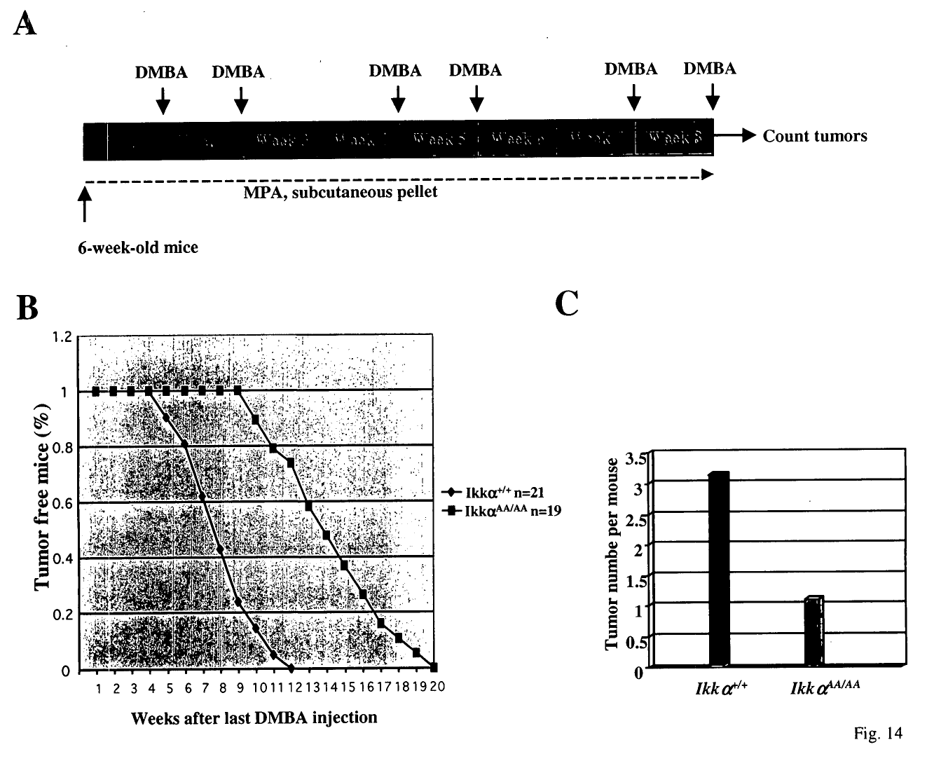

We have also studied the effect of the Ikkα^{AA} mutation on carcinogen-induced mammary cancer. The fifth generation of a backcross of Ikkα^{AA/AA} mice to the BALB/c background was used for this experiment. Six weeks old wt and Ikkα^{AA/AA} mice were given subcutaneous slow-release pellet of MPA (medroxyprogesterone acetate) followed by oral gavage of DMBA (7,12-dimethylbenzaanthracene) as illustrated in FIG. 14A. Mammary tumor formation was monitored twice a week after the last injection of DMBA. Again, we found that Ikkα^{AA} mutation delays tumor onset and reduces tumor incidence (FIG. 14B, C). In the wt group, tumors started to appear after 5 weeks and 50% of mice had developed tumors by 7 weeks, while no tumors were detected before 10 weeks in Ikkα^{AA/AA} mice and it took 13.5 weeks for 50% of these mice to develop tumors (FIG. 14B). Tumor numbers were also decreased in the Ikkα^{AA/AA} mice, averaging 1.1 tumors per mouse vs 3.1 tumors per mouse for the wt (FIG. 14C). These results suggest that IKKα is one of the signaling pathways involved in chemically-induced mammary carcinogenesis.

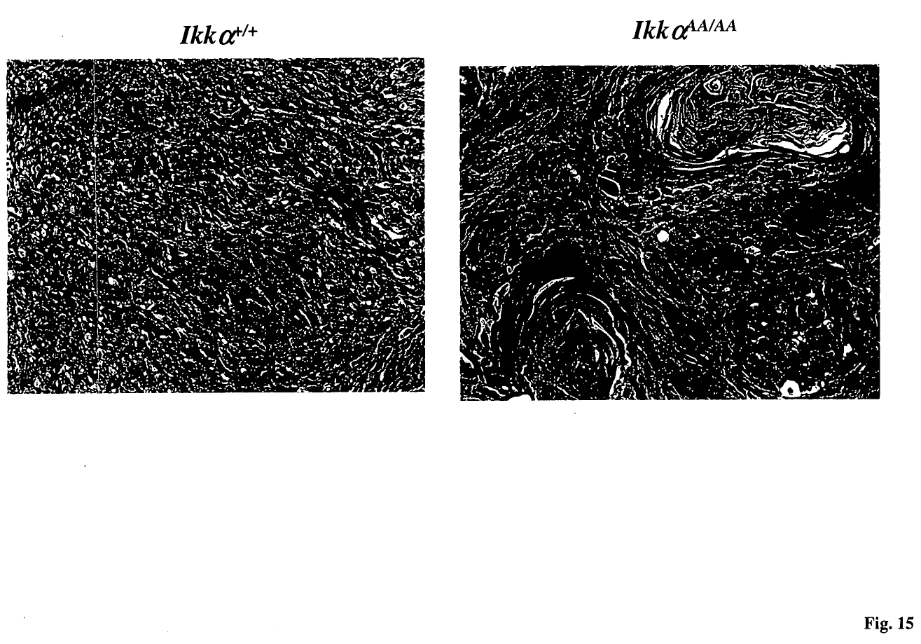

Histologically, unlike the “signature” phenotype characterizing the ErbB2-inducing tumors, DMBA induced tumors display a variety of phenotypes whose spectrum is influenced by the Ikkα^{AA} mutation. The majority of the tumors derived from the wt mice (18-20 out of 30 examined by HE staining and microscopy) show sheets of undifferentiated tumor cells, while 75% of tumors derived from Ikkα^{AA/AA} mice (9 out 12 examined) show squamous metaplasia, with keratin swirls being the most prominent feature (FIG. 15). Keratin swirls were seen only in 13% of the wt tumors. Since keratinization is commonly seen in Wnt/b-catenin-induced tumors our results suggest a bias in selection for tumors with activated b-catenin signaling in Ikkα^{AA/AA} mice, suggesting that the Ikkα^{AA} mutation might block other pathways that contribute to tumor formation in wt mice, but leave the Wnt/b-catenin pathway unirnpeded. in support of this hypothesis, we find that the Ikkα^{AA} mutation does not affect the incidence of mammary carcinoma induced by an MMTV-Wnt1 transgene.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiment, it should be understood that the invention as claimed should not be unduly limited to such specific embodiment. Indeed, various modifications of the described modes for carrying out the invention which are obvious to those skilled in the art and in fields related thereto are intended to be within the scope of the present invention.

1. A method for reducing mammary epithelial cell proliferation, comprising:
a) providing:
i) mammary epithelial cells; and
ii) an agent that specifically reduces IKKα: kinase activity;
b) administering said agent to said mammary epithelial cells to produce treated mammary epithelial cells such that mammary epithelial cell proliferation in said treated mammary epithelial cells is reduced.

2. The method of claim 1, wherein said mammary epithelial cells are in vitro.

3. The method of claim 1, wherein said mammary epithelial cells are in vivo in a subject.

4. The method of claim 3, wherein said subject is human.

5. The method of claim 3, wherein said mammary epithelial cells are in a tissue comprising a mammary tumor.

6. The method of claim 5, wherein said tumor is malignant.

7. The method of claim 6, wherein said malignant tumor is metastatic.

8. The method of claim 3, wherein said subject is suspected of being capable of developing a mammary tumor in a tissue.

9. The method of claim 3, wherein said administering is before manifestation of one or more symptoms of a mammary tumor.

10. The method of claim 3, wherein said administering is concomitant with manifestation of one or more symptoms of a mammary tumor.

11. The method of claim 3, wherein said administering is after manifestation of one or more symptoms of a mammary tumor.

12. The method of claim 3, wherein said administering is selected from parenteral, oral, intraperitoneal, sublingual, and into said tumor.

13. The method of claim 12, wherein said parenteral administering is selected from subcutaneous, intravenous, intramuscular, intrastemal injection, and infusion.

14. A method for reducing a mammary tumor in a subject, comprising:
a) providing:
i) a subject; and
ii) an agent that specifically reduces IKKα kinase activity;
b) administering said agent to said subject to produce a treated subject such that a mammary tumor in said treated subject is reduced.

15. The method of claim 14, wherein said subject is suspected of being capable of developing a mammary tumor in a tissue.

16. The method of claim 14, wherein said subject comprises a mammary tumor in a tissue.

17. A method for detecting tumor mammary epithelial cells in a sample, comprising detecting increased IKKα kinase activity in said tumor mammary epithelial cells or in said sample compared to IKKα kinase activity in control cells or in a control sample.

18. A method for detecting tumor mammary epithelial cells in a sample, comprising:
a) providing:
i) a sample suspected of comprising tumor mammary epithelial cells; and
ii) an anti-phosphoserine antibody;
b) administering said antibody to said sample such that said antibody binds to phosphoserine in an IKKα kinase domain to produce a treated sample; and
c) detecting increased binding of said antibody to said phosphoserine in said treated sample compared to binding of said antibody to said phosphoserine in a control sample, thereby detecting tumor mammary epithelial cells in said sample.

19. The method of claim 18, wherein said tumor mammary epithelial cells are in vitro.

20. The method of claim 18, wherein said tumor mammary epithelial cells are in vivo.

21. A method for detecting tumor mammary epithelial cells in a sample, comprising:
a) providing:
i) a sample suspected of comprising tumor mammary epithelial cells; and
ii) an agent that specifically binds to at least a portion of a molecule selected from IKKα polypeptide, IKKα kinase domain polypeptide, IKKα mRNA, and IKKα kinase domain mRNA;
b) administering said agent to said sample such that said agent specifically binds to said molecule to produce a treated sample comprising treated tumor mammary epithelial cells; and
c) detecting increased specific binding of said molecule to said agent in said treated sample or in said treated cells as compared to specific binding of said molecule to said agent in a control sample or in control cells, thereby detecting tumor mammary epithelial cells in said sample.

22. The method of claim 21, wherein said portion of IKKα polypeptide comprises an IKKα kinase domain.

23. The method of claim 21, wherein said portion of IKKα mRNA encodes a polypeptide comprising an IKKα kinase domain.

24. The method of claim 21, wherein said agent comprises an anti- phosphoserine antibody.

25. A method for screening a test compound as reducing mammary epithelial cell proliferation, comprising:
a) providing:
i) mammary epithelial cells;
ii) a test compound; and
b) administering said test compound to said mammary epithelial cells to produce treated mammary epithelial cells;
c) detecting a reduction in IKKα kinase activity in said treated mammary epithelial cells, thereby identifying said test compound as reducing mammary epithelial cell proliferation.

26. The method of claim 25, wherein said mammary epithelial cells are in vitro.

27. The method of claim 25, wherein said mammary epithelial cells are in vivo.

28. The method of claim 25, wherein said mammary epithelial cells are normal.

29. The method of claim 25, wherein said mammary epithelial cells are tumor.

30. A method for screening a test compound as reducing mammary epithelial cell proliferation, comprising:
a) providing:
i) a sample comprising IKKα or IKKα kinase domain;
ii) an IKKα substrate;
iii) a test compound; and
b) contacting said sample, said IKKα substrate, and said test compound such that said IKKα substrate is phosphorylated by said IKKα or said IKKα kinase domain; and
c) detecting a reduction in the level of phosphorylation of said IKKα substrate in the presence of said test compound compared to in the absence of said test compound, thereby identifying said test compound as reducing mammary epithelial cell proliferation.

31. The method of claim 30, wherein said IKKα kinase domain is recombinant.

32. The method of claim 30, wherein said IKKα in said sample is recombinant.

33. A method for screening a test compound as reducing mammary epithelial cell proliferation, comprising:
a) providing:
i) a sample comprising IKKα;
ii) an anti-phosphoserine antibody;
iii) a test compound; and
b) contacting said sample, said antibody, and said test compound such that said antibody binds to said IKKα; and
c) detecting a reduction in the level of binding of said antibody to said IKKc: in the presence of said test compound compared to in the absence of said test compound, thereby identifying said test compound as reducing mammary epithelial cell proliferation.

34. A method for screening a test compound as specifically reducing IKKcc kinase activity, comprising:
a) providing:
i) a first transgenic mouse expressing a neu oncogene;
ii) a second transgenic mouse expressing:
1) a neu oncogene; and
2) a modified IKKα kinase domain that comprises replacing the Serine residues at amino acids 176 and 180 of iKKoc kinase polypeptide with Alanine, wherein said modified IKKαc kinase domain has reduced IKKαc kinase activity; and
iii) a test compound; and
b) administering said test compound to said first transgenic mouse to produce a first treated transgenic mouse and to said second transgenic mouse to produce a second treated transgenic mouse; and
c) detecting a reduction in mammary tumors in said first treated transgenic mouse that is not greater than a reduction in mammary tumors in said second treated transgenic mouse, thereby identifying said test compound as specifically reducing IKKα kinase activity.

35. The method of claim 34, further comprising step d) identifying said test compound as reducing mammary epithelial cell proliferation.


SEQUENCE LISTING (15 sequences)

SEQ ID NO: 1 - DNA, 45 bp, Artificial Sequence
  Synthetic
gatgttgatcaaggagcgctctgtacagcgtttgtgggaacattg

SEQ ID NO: 2 - DNA, 45 bp, Artificial Sequence
  Synthetic
caatgttcccacaaacgctgtacagagcgctccttgatcaacatc

SEQ ID NO: 3 - DNA, 23 bp, Artificial Sequence
  Synthetic
ccggaagctactcaacaaaccag

SEQ ID NO: 4 - DNA, 18 bp, Artificial Sequence
  Synthetic
ccatcatggctgatgcaa

SEQ ID NO: 5 - DNA, 17 bp, Artificial Sequence
  Synthetic
gataccgtaaagcacga

SEQ ID NO: 6 - DNA, 23 bp, Artificial Sequence
  Synthetic
ggatccgatatctgcatgaaaac

SEQ ID NO: 7 - DNA, 30 bp, Artificial Sequence
  Synthetic
caatgttcccacaaaagatgtacagagact

SEQ ID NO: 8 - DNA, 30 bp, Artificial Sequence
  Synthetic
caatgttcccacaaacgctgtacagagcgc

SEQ ID NO: 9 - DNA, 29 bp, Artificial Sequence
  Synthetic
ggtaaatggctactaaaccgaacttctcc

SEQ ID NO: 10 - DNA, 27 bp, Artificial Sequence
  Synthetic
tacccatacgatgttccagattacgct

SEQ ID NO: 11 - DNA, 21 bp, Artificial Sequence
  Synthetic
tcataacgtcagacgctggcc

SEQ ID NO: 12 - DNA, 2273 bp, Homo sapiens
tcgacggaacctgaggccgcttgccctcccgccccatggagcggcccccggggctgcggccgggcgcgggcgggccctgggagatgcgggagcggctgggcaccggcggcttcgggaacgtctgtctgtaccagcatcgggaacttgatctcaaaatagcaattaagtcttgtcgcctagagctaagtaccaaaaacagagaacgatggtgccatgaaatccagattatgaagaagttgaaccatgccaatgttgtaaaggcctgtgatgttcctgaagaattgaatattttgattcatgatgtgcctcttctagcaatggaatactgttctggaggagatctccgaaagctgctcaacaaaccagaaaattgttgtggacttaaagaaagccagatactttctttactaagtgatatagggtctgggattcgatatttgcatgaaaacaaaattatacatcgagatctaaaacctgaaaacatagttcttcaggatgttggtggaaagataatacataaaataattgatctgggatatgccaaagatgttgatcaaggaagtctgtgtacatcttttgtgggaacactgcagtatctggccccagagctctttgagaataagccttacacagccactgttgattattggagctttgggaccatggtatttgaatgtattgctggatataggccttttttgcatcatctgcagccatttacctggcatgagaagattaagaagaaggatccaaagtgtatatttgcatgtgaagagatgtcaggagaagttcggtttagtagccatttacctcaaccaaatagcctttgtagtttaatagtagaacccatggaaaactggctacagttgatgttgaattgggaccctcagcagagaggaggacctgttgaccttactttgaagcagccaagatgttttgtattaatggatcacattttgaatttgaagatagtacacatcctaaatatgacttctgcaaagataatttcttttctgttaccacctgatgaaagtcttcattcactacagtctcgtattgagcgtgaaactggaataaatactggttctcaagaacttctttcagagacaggaatttctctggatcctcggaaaccagcctctcaatgtgttctagatggagttagaggctgtgatagctatatggtttatttgtttgataaaagtaaaactgtatatgaagggccatttgcttccagaagtttatctgattgtgtaaattatattgtacaggacagcaaaatacagcttccaattatacagctgcgtaaagtgtgggctgaagcagtgcactatgtgtctggactaaaagaagactatagcaggctctttcagggacaaagggcagcaatgttaagtcttcttagatataatgctaacttaacaaaaatgaagaacactttgatctcagcatcacaacaactgaaagctaaattggagttttttcacaaaagcattcagcttgacttggagagatacagcgagcagatgacgtatgggatatcttcagaaaaaatgctaaaagcatggaaagaaatggaagaaaaggccatccactatgctgaggttggtgtcattggatacctggaggatcagattatgtctttgcatgctgaaatcatggggctacagaagagcccctatggaagacgtcagggagacttgatggaatctctggaacagcgtgccattgatctatataagcagttaaaacacagaccttcagatcactcctacagtgacagcacagagatggtgaaaatcattgtgcacactgtgcagagtcaggaccgtgtgctcaaggagctgtttggtcatttgagcaagttgttgggctgtaagcagaagattattgatctactccctaaggtggaagtggccctcagtaatatcaaagaagctgacaatactgtcatgttcatgcagggaaaaaggcagaaagaaatatggcatctccttaaaattgcctgtacacagagttctgcccgctctcttgtaggatccagtctagaaggtgcagtaacccctcagacatcagcatggctgcccccgacttcagcagaacatgatcattctctgtcatgtgtggtaactcctcaagatggggagacttcagcacaaatgatagaagaaaatttgaactgccttggccatttaagcactattattcatgaggcaaatgaggaacagggcaatagtatgatgaatcttgattggagttggttaacagaatga

SEQ ID NO: 13 - protein, 745 aa, Homo sapiens
MERPPGLRPGAGGPWEMRERLGTGGFGNVCLYQHRELDLKIAIKSCRLELSTKNRERWCHEIQIMKKLNHANVVKACDVPEELNILIHDVPLLAMEYCSGGDLRKLLNKPENCCGLKESQILSLLSDIGSGIRYLHENKIIHRDLKPENIVLQDVGGKIIHKIIDLGYAKDVDQGSLCTSFVGTLQYLAPELFENKPYTATVDYWSFGTMVFECIAGYRPFLHHLQPFTWHEKIKKKDPKCIFACEEMSGEVRFSSHLPQPNSLCSLIVEPMENWLQLMLNWDPQQRGGPVDLTLKQPRCFVLMDHILNLKIVHILNMTSAKIISFLLPPDESLHSLQSRIERETGINTGSQELLSETGISLDPRKPASQCVLDGVRGCDSYMVYLFDKSKTVYEGPFASRSLSDCVNYIVQDSKIQLPIIQLRKVWAEAVHYVSGLKEDYSRLFQGQRAAMLSLLRYNANLTKMKNTLISASQQLKAKLEFFHKSIQLDLERYSEQMTYGISSEKMLKAWKEMEEKAIHYAEVGVIGYLEDQIMSLHAEIMGLQKSPYGRRQGDLMESLEQRAIDLYKQLKHRPSDHSYSDSTEMVKIIVHTVQSQDRVLKELFGHLSKLLGCKQKIIDLLPKVEVALSNIKEADNTVMFMQGKRQKEIWHLLKIACTQSSARSLVGSSLEGAVTPQTSAWLPPTSAEHDHSLSCVVTPQDGETSAQMIEENLNCLGHLSTIIHEANEEQGNSMMNLDWSWLTE

SEQ ID NO: 14 - DNA, 3466 bp, Mus musculus
gggaccggccttagaccggcggcgttgcctgaggcggctggcgctcccgccccatggagcggcccccggggctgcggccgggcgcgggcggcccctgggagatgcgggaacggcttggcaccggcggtttcgggaacgtcagtctgtaccagcaccgggaacttgatctcaaaatagcaattaagtcttgtcgtttagagctaagttccaaaaacagagagcgatggtgccatgaaatccagatcatgaaaaagttggaccatgcgaatgttgtaaaggcctgtgatgtccctgaggaattgaactttttaattaacgatgtgcctcttctggcaatggagtactgttctggaggggacctccggaagctactcaacaaaccagaaaattgttgtggacttaaagaaagccagatactttctttactgagtgacataggatctgggatccgatatctgcatgaaaacaaaattatacatcgagatctaaaacctgaaaatatagttcttcaagatgttggtgggaagacaatacataaaataattgatttgggttatgccaaagatgttgatcaaggaagtctctgtacatcttttgtgggaacattgcagtatttggccccagagctctttgaaaataagccgtacacagccactgtggattattggagctttgggaccatggtgtttgaatgtattgctggatataggccttttttgcatcatctgcagccatttacctggcatgagaagattaagaagaaagatccaaagtgtatatttgcatgtgaagagatgactggagaagttcggtttagtagccatttacctcagccaaacagcctttgtagtttaatagtagagccaatggaaagctggctccaattgatgctgaattgggacccacagcagagagggggacctattgatcttactttgaagcagccaagatgttttgcattaatggatcacattctcaatttaaagatagtgcacatcctaaatatgacttctgcaaaaatcatttcttttctgttaccatgtgatgaaagtcttcattcactacagtctcgaattgagcgtgaaacaggaataaatacaggttctcaggagcttctgtcagagacagggatttctctggatcctcggaaaccagcctctcagtgtgttctagatggagttagaggctgtgatagctacatggtttatttgtttgataaaagtaagactgtatatgaaggaccatttgcatccagaagtttatctgattgtgtaaattatattgtacaagacagcaaaatacaactgccaattatacagctgcggaaagtatgggctgaagcagtgcactacgtatctgggctaaaggaagactacagcaggctcttccagggacaaagagcagcaatgttaagtcttcttagatataatgctaacttgacaaaaatgaagaatactttgatctcagcatcacagcaactcaaagctaaattggagttttttcgaaaaagcattcagcttgacttggagagatatagtgagcagatgacttatgggatatcttcagaaaaaatgttaaaagcatggaaagaaatggaagaaaaggccattcactattctgaggttggtgtcattggttatcttgaggatcaaattatgtctttgcacactgaaatcatggagctgcagaagagcccctacggacgacgccagggagacttgatggagtctctggagcagcgtgccattgatctctataagcagctaaagcacagacctcctgatcacttgtacagcgacagcacagagatggtgaagatcatcgtgcacaccgtgcagagtcaggaccgtgttctcaaggagctgtttggtcacctgagcaagttgttgggctgcaagcagaagattattgatctactccccaaggtggaagtggccctcagtaacatcaaagaagctgacaatactgtcatgtttatgcagggaaagaggcagaaagaaatttggcacctccttaaaattgcctgtacacagagttctgcccgctctcttgtaggatccagtctagaaggcacagtaacccctccagtatcagcatggctgccccctacattagcagaccgtgaacatcctctgacatgtgtggtaactcctcaagatggagagacgttagcacaaatgatagaagaaaatctgaactgtcttggccatttaagtactattattcgtgaagcaaatgaggaccagagcagtagtttgatgagtcttgattggagttggttagcagaatgactcgacactcgttcactgtcctggagcctacgaagctgttttgtcatttactccaaagtcatctttacttgctgaagccattcctcacttaccagtccgtgaggagatggctgtgatcggaaactacgagtgactttacaagcacagtagcttggtgttttgtttgtttctaataattatgatctctgaacagatagaattttatagcaaattagtgaaattaattattctttttaacaccgcaactaatgagggagatcattagtgacctgcttatcttataaaattggaaaaatactactactagtttagctgatgaaaaagataatcttctaaaggcctaaattttcggcataaggcccaacatggtattagtatacaggaatgaaaaattcacccagtgttcatttgaagtaaagttttatctatgggttttctgtggaagagactgctgacaagtaaaattgctcttcctgaagactaagcccagcctccttgtgttgctctcagcaagtgttcttcatggcatcacatggagtcagatgaatcccatctttaatcacacatttaatagagtccttttcctgtgtaaggggttggacttttgtgcctttgatatcagctgaccataatgaattgtgttgtgtgctatatgtatatgtatttaaggtgtacatttaataatatcaaagagaagatgcctgttaatttataatgtatttgaaagttgtattgtttttgcatttgtaaaaatgggttacttgtttaaacaatcttttatgtcttgtcatacaaattccaaagggtctgcattcctttatctgtaattacagtctcagaatccaagttctgaaaacaaggtatctattctgatctgacactggatctgcttatcccatttagtgtgaatattcattgatttatgtgtttgattattgggatgtgctgccacaggctctcttgaaggttgatgtagtgtggcgtatgcactgaattacctttctaaaatctgaacagttctcattctgaaacatctagacttaagggtttcagataaaagactgcggttctctgccttatgttaaataacttagaagatgttattttgtttgaaaaaatgtgaaatgcttttatattctagtttttcactttgcatattaaatgattttaaaatt

SEQ ID NO: 15 - protein, 745 aa, Mus musculus
MERPPGLRPGAGGPWEMRERLGTGGFGNVSLYQHRELDLKIAIKSCRLELSSKNRERWCHEIQIMKKLDHANVVKACDVPEELNFLINDVPLLAMEYCSGGDLRKLLNKPENCCGLKESQILSLLSDIGSGIRYLHENKIIHRDLKPENIVLQDVGGKTIHKIIDLGYAKDVDQGSLCTSFVGTLQYLAPELFENKPYTATVDYWSFGTMVFECIAGYRPFLHHLQPFTWHEKIKKKDPKCIFACEEMTGEVRFSSHLPQPNSLCSLIVEPMESWLQLMLNWDPQQRGGPIDLTLKQPRCFALMDHILNLKIVHILNMTSAKIISFLLPCDESLHSLQSRIERETGINTGSQELLSETGISLDPRKPASQCVLDGVRGCDSYMVYLFDKSKTVYEGPFASRSLSDCVNYIVQDSKIQLPIIQLRKVWAEAVHYVSGLKEDYSRLFQGQRAAMLSLLRYNANLTKMKNTLISASQQLKAKLEFFRKSIQLDLERYSEQMTYGISSEKMLKAWKEMEEKAIHYSEVGVIGYLEDQIMSLHTEIMELQKSPYGRRQGDLMESLEQRAIDLYKQLKHRPPDHLYSDSTEMVKIIVHTVQSQDRVLKELFGHLSKLLGCKQKIIDLLPKVEVALSNIKEADNTVMFMQGKRQKEIWHLLKIACTQSSARSLVGSSLEGTVTPPVSAWLPPTLADREHPLTCVVTPQDGETLAQMIEENLNCLGHLSTIIREANEDQSSSLMSLDWSWLAE